(19) United States
(12) Patent Application Publication
(10) Pub. No.: US 2004/0005644 A1
(43) Pub. Date: Jan. 8, 2004

(54) Method and composition for detection and treatment of breast cancer

(76) Inventors: Yan A. Su, Bethesda, MD (US); Jun Yang, Hinsdale, IL (US)

Correspondence Address: DORSEY & WHITNEY LLP, 1001 PENNSYLVANIA AVENUE, N.W., SUITE 400 SOUTH, WASHINGTON, DC, 20004, US

(21) Appl. No.: 10/373,801

(22) Filed: Feb. 27, 2003

Related U.S. Application Data
(60) Provisional application No. 60/359,999, filed on Feb. 28, 2002.

(51) Int. Cl. G01N 33/574

(52) U.S. Cl. 435/7.23

(57) ABSTRACT

The present invention provides a method for the detection of breast cancer using breast by measuring expression levels of breast cancer specific marker (BCSM) genes, and in particular the level of polynucleotides transcribed from and polypeptides encoded by the BCSM genes. The present invention also provide a method for the treatment and/or prevention of breast cancer by modulating the activity of BCSM genes or the products of BCSM genes.

### RELATED APPLICATION

This application is related to U.S. Provisional Application Serial No. 60/359,999, filed Feb. 28, 2002.

### TECHNICAL FIELD

The present invention relates generally to the detection and treatment of cancer, and in particular breast cancer. The invention specifically relates to breast cancer-specific genes (BCSG), and to polynucleotides transcribed from and polypeptides encoded by the BCSGs. Such polynucleotides and polypeptides may be used for the detection and treatment of breast cancer.

### BACKGROUND

Breast cancer is the second leading cause of cancer-related deaths of women in North America. Although advances have been made in detection and treatment of the disease, breast cancer remains the second leading cause of cancer-related deaths in women, affecting more than 180,000 women in the United States each year.

Approximately 10% of all breast cancers are currently classified as strongly familial with many of these appearing to be caused by mutations in the hereditary breast cancer genes BRCA1 or BRCA2. However, at least one-third of breast cancers that seem to run in families are not linked to BRCA1 or BRCA2, suggesting the existence of an additional hereditary breast cancer gene or genes. Recently, structural and functional studies of cancer cell lines and tissues have demonstrated the involvement of many genetic loci and genes in the development of human breast cancer. Cytogenesis and loss of heterozygocity (LOH) studies have led to the discoveries of alterations in human chromosomes including 1p, 1q, 3p, 6q, 7q, 11p, 13q, 16q, 17p, 17q, and 18q, at frequencies as high as 20-60%. Thus, multiple genes are involved in the development of extensively heterogeneous breast cancers.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. (See, e.g., Porter-Jordan and Lippman, Breast Cancer 8:73-100, 1994). However, the use of established markers often leads to a result that is difficult to interpret, and the high mortality observed in breast cancer patients indicates that improvements are needed in the treatment, diagnosis and prevention of the disease.

Accordingly, there is a need in the art for improved methods for therapy and diagnosis of breast cancer. The identification of expression profiles and differentially expressed genes in the genomic scale would greatly facilitates the molecular classification of tumors and discovery of genes that are causally related to breast cancer development.

### SUMMARY OF THE INVENTION

The present invention provides compositions and methods for the diagnosis and treatment of breast cancer. Specifically, the present invention discloses genes that are differentially expressed in breast cancer cell lines and breast cancer tissue samples as compared to control cell lines and normal tissue samples, the polynucleotides transcribed from these genes (SEQ ID NOS:1-19), and the polypeptides encoded by these polynucleotides (SEQ ID NOS:20-38). The differentially expressed genes are designated as breast cancer specific genes (BCSG). The polynucleotides transcribed from and the polypeptides encoded by the BCSGs are designated as breast cancer specific markers (BCSM).

In one aspect, the present invention provides a method for diagnosing and monitoring breast cancer by comparing the expression levels of one or more BCSM in biological samples from a subject to control samples.

In a related aspect, the present invention provides a kit for diagnosing breast cancer. The kit comprises at least one of the following (1) polynucleotide probe that specifically hybridizes to a polynucleotide transcribed from a BCSG, and (2) an antibody capable of immunospecific binding to a BCSM.

In another aspect, the present invention provides a pharmaceutical composition for the treatment of breast cancer. The pharmaceutical composition comprises a pharmaceutically acceptable carrier and at least one of the following: (1) a BCSM or a functional variant of a BCSM, (2) an antibody directed against a BCSM or its functional variant, (3) a vaccine generated using a BCSM or its variant, (4) an agent that modulate an expression level of a BCSG or an activity of a BCSM.

In a related aspect, the present invention provides a method for treating breast cancer in a patient with the pharmaceutical composition described above. The patient may be afflicted with breast cancer, in which case the methods provide treatment for the disease. The patient may also be considered at risk for breast cancer, in which case the methods provide prevention for cancer development.

In another embodiment, the present invention provides methods for screening anti-breast cancer agents based on the agents interaction with the BCSMs, or the agents' effect on the expression of the BCSGs.

In another embodiment, the present invention provides animals transgenic for one or more of the BCSGs, or a knockout animal in which one or more of the BCSGs is disrupted. These animals may be used to study the relevance of BCSGs to the development of breast cancer.

In another embodiment, the present invention provides host cells harboring a transfected BCSG. These cells may be used for the treatment of breast cancer.

Other aspects of the invention will become apparent to the skilled artisan by the following description of the invention.

### BRIEF DESCRIPTION OF FIGURES

The inventions of this application are better understood in conjunction with the following drawings, in which:

FIG. 1 shows patterns of gene expression in MDA-MB-231 (breast cancer) and MDA/H6 (non-tumorigenic) cell lines. (A) Phosphor images of gene filters. Five gene filters (gf200, gf201, gf202, gf203, gf211) were hybridized first with radioactively labeled cDNA from MDA-MB-231 cells and then with that from MDA/H6 cells. (B) Color images derived from the alignment of radioactive images. (C) A scatter plot of expression intensities of 25,985 genes in MDA-MB-231 and MDA/H6. Each dot represents a gene plotted at the coordinate of its two expression intensities on a log-scale. The genes with the equal intensities are condensed along a diagonal line. (D) The original and color images of 30 genes up-regulated in MDA/H6 with low, medium, and high levels of the expression. Three equally expressed genes were indicated. Red: up-regulated in MDA/H6; green: down regulated in MDA/H6; yellow: no changes.

FIG. 2 shows analysis of images and expression data on the customized microarrays. (A and B): The images of two sets of 768 genes on the same glass slide. The image A shows the identical patterns with the image B. (i and i′): the gene encoding for prostaglandin endoperoxide synthase 2; (ii and ii′) the gene for 3-hydroxymethyl-3-methylglutaryl-Coenzyme A lyase; (iii and iii′) the gene for ribosomal protein L10. (C and D) Statistical analysis of the expression ratios of 202 informative genes between MDA-MB-231 and MDA/H6 were detected by two sets of genes (images A and B) on Slide 1 (C) and on Slide 2 (D). (E) The average ratios of the gene expression from Slide 1A and 1B were plotted against the average ratios from Slide 2A and 2B. The linear regression and Pearson coefficient of correlation were computed from the scatter plots that are on log-scale. The strong linear relations and high values of Pearson coefficient of correlation (r) are indicated in each comparison. “x”: an gene expression ratio between MDA-MB-231 and MDA/H6 on x-axis; “y”: the ratio between these two samples on the y-axis corresponding to a given “x”.

FIG. 3 depicts clustering of the gene expression data. (A) Multidimensional scaling analysis. 3-dimentional plot of all 15 cancer samples showing two identical MDA-MB-231 samples (MB231 1 and 2, green), the most dissimilar melanoma sample (MelTis in yellow), three most similar breast cancer samples (BT20, ZR75-1, and BT474 in red) and others in blue. (B and C) Gene and sample dendrograms from the hierarchical clustering analysis reveal co-regulated genes and relationship among the samples. Two MDA-MB-231 samples are essentially identical (r=0.982). Human melanoma specimen (MelTis) is the most dissimilar to MDA-MB-231 (r=0.325). Twelve breast cancer samples are clustered in the center. Three most similar samples were BT20, BT474 and ZR-75-1 (r=0.796). The numbers on the nodes indicate the values of Pearson coefficient of correlation. (D) Nine genes with significantly up-regulated expression (≧2 folds) in at least 10 of 13 breast cancer samples. These nine genes were also over-expressed in the metastatic melanoma. (E) Ten genes with significantly down-regulated expression (≧0.5 folds) in at least 10 of 13 breast cancer samples. The clone ID and the gene names are listed on the left and the right of the panels, respectively.

FIG. 4 shows the correlation of thrombomodulin (THBD) RNA expression to THBD protein expression as measured by cDNA microarrays and Western blots, respectively. (A) The THBD RNA levels in 13 breast cancer cell lines measured by cDNA microarrays using MDA/H6 as the reference. The values of the intensity means (I.M.), the intensity standard deviations (I.D.), and the calibrated (Cal.) ratios for the test samples and the reference are the averages derived from the cDNA microarray images A and B on each slide (see FIG. 2). The green filled box and Cal. ratio indicate the decrease of the TH gene in a test sample relative to the corresponding MDA/H6 reference. (B) Western blot of the whole cell lysates from the breast cancer cell lines: MDA/H6 (lane 1), MB231 (lane 2), MB436 (lane 3), MB453 (lane 4) and BT549 (lane5), using the antibody against THBD (top panel) and the antibody against actin (bottom panel) as a control for loading error. Ninety-eight kilodaltons (kD) and 43 kD indicate the THBD protein and actin protein, respectively. The protein intensities in the lanes 2, 3, 4, and 5 approximate the RNA levels in the corresponding breast cancer cells: MB231, MB231, MB436, MB453 and BT549. The lane 1 shows the THBD protein intensity in the non-tumorigenic breast cancer cell line MDA/H6 that displays the highest RNA level in all the cell lines.

FIG. 5 show representative images of the pathological sections of normal and cancerous breast tissues from Case 1 (A) and Case 6 (B) in Table 6. (A1) A section shows normal breast tissue, of which the mammary epithelial cells were stained to brown (positive) by the TH antibody (A2). (A3) A tissue section shows infiltrating ductal carcinoma, of which the cancer cells were not stained by the TH antibody (A4). (B1) A section shows normal mammary epithelial tissue (indicated by the horizontal arrowheads) and infiltrating ductal carcinoma (indicated by the vertical arrowheads); (B2) Normal mammary epithelial cells were stained to brown (positive) by the TH antibody; in contrast, the cancer cells were not. Magnification: (A1 and A2), 100-fold; (A3 and A4), 200-fold; (B1 and B2), 40 fold.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that the specific nomenclature is not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The present invention is generally directed to compositions and methods for the diagnosis, treatment, and prevention of breast cancer. The present invention is based on the discovery of transcribed polynucleotides that are either over-expressed or under-expressed in human breast cancer cell line MDA-MB-231 as related to the non-tumorigenic derivative cell line MDA/H6.

Definitions and Terms

To facilitate an understanding of the present invention, a number of terms and phrases are defined below:

As used herein, the term “breast cancer specific gene (BCSG)” refers to a gene that is over-expressed by at least two-fold (i.e. ≧200% of normal) or under-expressed by at least two-fold (i.e., ≦50% of normal) in breast cancer tissue or cell lines relative to normal tissue or cell lines. Specifically, BCSG refers to the genes listed in Table 1 and the alleles of these genes.

As used herein, “a breast cancer-specific marker (BCSM)” refers to a polynucleotide transcribed from a BCSG or a polypeptide translated from such a polynucleotide. BCSM and “BCSG product” are used interchangeably.

As used herein, “a BCSM and its variants” refers to variants of a polynucleotide transcribed from a BCSG and variants of a polypepetide encoded by a BCSG.

As used herein, the terms “polynucleotide” “nucleic acid” and “oligonucleotide” are used interchangeably, and include polymeric forms of nucleotides of any length, either deoxyribonucleotides or ribonucleotides, or analogs thereof. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers.

As used herein, the terms “variants of a polynucleotide” refers to polynucleotides that, as a result of the degeneracy of the genetic code, encode the same polypeptide as described herein. Some of these polynucleotides bear minimal homology to the nucleotide sequence of any native gene. Nonetheless, polynucleotides that vary due to differences in codon usage are specifically contemplated by the present invention. A variant may contain one or more substitutions, additions, deletions and/or insertions such that the activity or immunogenicity of the encoded polypeptide is not substantially enhanced or diminished, relative to a native polypeptide.

Variants of a polynucleotide may also be substantially homologous to a native gene, or a portion or complement thereof. Such polynucleotide variants are capable of hybridizing under moderately stringent conditions to a naturally occurring DNA sequence encoding a native breast tumor protein (or a complementary sequence). Suitable moderately stringent conditions include prewashing in a solution of 5×SSC, 0.5% SDS. 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS. Standard hybridization techniques are described in Sambrook et al., Molecular Cloning. A Laboratory Manual, Cold Spring Harbor Laboratories, Cold Spring Harbor, N.Y., 1989.

As used herein, a “variant of a polypeptide” is a polypeptide that differs from a native polypeptide in one or more substitutions, deletions, additions and/or insertions, such that the functionality of the polypeptide is not substantially enhanced or diminished. In other words, a variant retains the biological activities of the native peptide. The biological activities of the variant may be enhanced or diminished by less than 50%, preferably less than 20%, relative to the native polypeptide. Similarly, the ability of a variant to react with antigen-specific antisera may be enhanced or diminished by less than 50%, preferably less than 20%, relative to the native polypeptide. Such variants may generally be identified by modifying one of the above polypeptide sequences and evaluating the reactivity of the modified polypeptide with antigen-specific antibodies or antisera as described herein.

Preferably, a variant polypeptide contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Variants may also be modified by, for example, the deletion or addition of amino acids that have minimal influence on the immunogenicity, secondary structure and hydropathic nature of the polypeptide.

Polypeptide variants preferably exhibit at least about 70%, more preferably at least about 90% and most preferably at least about 95% homology to the original polypeptide.

A polypeptide variant also include a polypeptides that is modified from the original polypeptides by either natural processes, such as posttranslational processing, or by chemical modification techniques which are well known in the art. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

As used herein, a “biologically active portion” of a polypeptide encoded by a BCSG includes a fragment of the polypeptide comprising amino acid sequences derived from the original polypeptide, which include fewer amino acids than the full length polypeptide, and exhibit at least one activity of the full length polypeptide. Typically, biologically active portions comprise a domain or motif with at least one activity of the full length polypeptide. A biologically active portion of a polypeptide encoded by a BCSG can be a polypeptide which is, for example, 10, 25, 50, 100, 200 or more amino acids in length.

As used herein, an “immunologenic portion” or “epitope” of a polypeptide encoded by a BCSG includes a fragment of the original polypeptide comprising amino acid sequences sufficiently homologous to or derived from the amino acid sequence of the original polypeptide, which include fewer amino acids than the full length polypeptide and can be used as an antigen to stimulate anti-BCSG peptide immune response.

As used herein, the term “modulation” includes, in its various grammatical forms (e.g., “modulated”, “modulation”, “modulating”, etc.), up-regulation, induction, stimulation, potentiation, inhibition, down-regulation, or suppression.

As used herein, the term “control sequences” or “regulatory sequences” refers to DNA sequences necessary for the expression of an operably linked coding sequence in a particular host organism. The term “control/regulatory sequence” is intended to include promoters, enhancers and other expression control elements (e.g., polyadenylation signals). Control/regulatory sequences include those which direct constitutive expression of a nucleotide sequence in many types of host cells and those which direct expression of the nucleotide sequence only in certain host cells (e.g., tissue-specific regulatory sequences).

A nucleic acid sequence is “operably linked” to another nucleic acid sequence when it is placed into a functional relationship with another nucleic acid sequence. For example, coding sequences of a BCSG can be operably linked to the regulatory sequences in a manner which allows for expression of the BCSG (e.g., in an in vitro transcription/translation system or in a host cell when the vector is introduced into the host cell). DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “immunospecific binding” refers to the specific binding of an antibody to an antigen at an affinity that is at least 10⁵M⁻¹.

As used herein, the term “biomolecules” refers to molecules having a bioactivity in a mammal. Examples of biomolecules include, but are not limited to, amino acids, nucleic acids, lipids, carbohydrates, polypeptides, polynucleotides, and polysaccsharides.

Breast Cancer Specific Genes

Breast cancer consists of extensively heterogeneous tumors and individual tumor cells may have specific genetic defects that determine gene expression patterns. Identification of expression profiles of multiple cancer samples may reveal genes and their expression patterns that consist of portions specific to the individual samples and common to most, if not all, samples studied. The common expression patterns might represent a common “passage” through which the cells evolve from one status to another. Although the high throughput technology DNA microarray is very useful to reveal genome-wide gene expression profiles, high density microarrays of thousands of genes are currently too expensive for routine research activities in majority laboratories.

The present invention uses an alternative approach to combine high density gene filters and low-cost high quality microarrays to study genome-wide gene expression. Gene expression profiles between the parental metastatic breast cancer cell line MDA-MB-231 and the chromosome 6-mediated suppressed non-tumorigenic derivative cell line MDA/H6 were initially compared using gene filters with 19,592 unique human genes/6,393 controls and radioactive detection technique. Six hundred and fifty-one genes were found to have more than 800 radioactive signal intensities and more than 2-fold changes in expression between the parental breast cancer cell line MDA-MB-231 and the non-tumorigenic cell line MDA/H6.

The 651 differentially expressed genes were further examined using customized DNA microarrays and fluorescence detection techniques. Since gene expression levelsin the same cells detected by microarrays can be affected by many factors including cell culture conditions, RNA purification, cDNA labeling methods and the quality of microarrays, high quality microarrays were used in the present invention to reduce the variance that could otherwise be introduced by different microarray slides. Strong positive linear relations with high values of Pearson coefficient of correlation were obtained between 2 sets of genes on the same slides and between the genes on the different slides, demonstrating the consistency of the microarrays and reproducibility of the experiments. The microarray analysis revealed 202 genes that were expressed differentially in breast cancer cell lines (n=10) and clinical breast cancer specimens (n=3) as related to normal tissues. The genes identified by the microarray and their expression profiles are listed in Tables 1 and 2, respectively.

TABLE 1
Genes with informative expression profiles in breast cancer cell lines

| Clone ID | Gene Name | Title | Plate Position |
|---|---|---|---|
| 23185 | TNC | hexabrachion (tenascin C, cytotactin) | LCC9d11 |
| 23831 | ALDOC | aldolase C, fructose-bisphosphate | LCC1e11 |
| 26617 | ALCAM | activated leucocyte cell adhesion molecule | LCC2b1 |
| 26711 | NCBP2 | nuclear cap binding protein subunit 2, 20 kD | LCC1g10 |
| 28098 | LOC57862 | clones 23667 and 23775 zinc finger protein | LCC1e5 |
| 28116 | karyopherin a2 | karyopherin alpha 2 (RAG cohort 1, importin alpha 1) | LCC9e1 |
| 30476 | ESTs | ESTs | LCC9d8 |
| 32517 | FLJ10509 | hypothetical protein FLJ10509 | LCC8e12 |
| 33949 | PRPSAP1 | phosphoribosyl pyrophosphate synthetase-associated protein 1 | LCC1d8 |
| 36191 | Fibronectin 1 | fibronectin 1 | LCC9d10 |
| 39884 | IMPDH1 | IMP (inosine monophosphate) dehydrogenase 1 | LCC8a8 |
| 40026 | SLC25A4 | solute carrier family 25 (mitochondrial carrier; adenine nucleotide translocator), member 4 | LCC8g5 |
| 44178 | TEGT | testis enhanced gene transcript | LCC9d7 |
| 44255 | RPML3 | ribosomal protein, mitochondrial, L3 | LCC8a7 |
| 45641 | MAP2K3 | mitogen-activated protein kinase kinase 3 | LCC3a10 |
| 45801 | ESTs | ESTs | LCC5b5 |
| 49496 | AMID | programmed cell death 8 (apoptosis-inducing factor) | LCC9a12 |
| 49553 | ARF4L | ADP-ribosylation factor 4-like | LCC8g6 |
| 49987 | GRIA2 | glutamate receptor, ionotropic, AMPA 2 | LCC8e6 |
| 51718 | ESTs | ESTs | LCC9b3 |
| 66686 | RPL10 | ribosomal protein L10 | LCC1a11 |
| 71101 | PROCR | protein C receptor, endothelial (EPCR) | LCC2h2 |
| 79710 | KIAA0174 | KIAA0174 gene product | LCC2e10 |
| 80910 | SLC1A5 | solute carrier family 1 (neutral amino acid transporter), member 5 | LCC2e8 |
| 108667 | SF3A1 | splicing factor 3a, subunit 1, 120 kD | LCC8b6 |
| 112576 | ESTs | ESTs | LCC3e1 |
| 114101 | ESTs | ESTs | LCC9c8 |
| 127519 | POH1 | 26S proteasome-associated pad1 homolog | LCC1f11 |
| 127821 | ACP5 | acid phosphatase 5, tartrate resistant | LCC2a8 |
| 128243 | ADK | adenosine kinase | LCC2b5 |
| 129585 | EST(Metallothionein2) | EST, Moderately similar to Cd-7 Metallothionein-2 [H. sapiens] | LCC3d9 |
| 131563 | FLJ13443 | Homo sapiens cDNA FLJ13443 fis, clone PLACE1002853 | LCC4a1 |
| 134495 | FLJ10976 | Homo sapiens cDNA FLJ10976 fis, clone PLACE1001399 | LCC4a10 |
| 135083 | GRP58 | glucose regulated protein, 58 kD | LCC8c10 |
| 136798 | Fibronectin 1 | fibronectin 1 | LCC9a5 |
| 138345 | PTP IVA | protein tyrosine phosphatase type IVA, member 1 | LCC9a6 |
| 139883 | ESTs | ESTs | LCC4b2 |
| 142586 | MCT-1 | MCT-1 protein | LCC4f6 |
| 144926 | ESTs | ESTs, Weakly similar to B0495.6 [C. elegans] | LCC3e5 |
| 147050 | PTGS2 | prostaglandin-endoperoxide synthase 2 (prostaglandin G/H synthase and cyclooxygenase) | LCC9d4 |
| 147338 | ESTs | ESTs | LCC9a7 |
| 163097 | MAM | melanoma adhesion molecule | LCC9d1 |
| 173554 | SFRS3 | splicing factor, arginine/serine-rich 3 | LCC6d8 |
| 191603 | TUBB | tubulin, beta polypeptide | LCC8a4 |
| 198871 | ESTs | ESTs | LCC9b6 |
| 201436 | | | LCC9c4 |
| 205185 | THBD | thrombomodulin | LCC1a7 |
| 207358 | SLC2A1 | solute carrier family 2 (facilitated glucose transporter), member 1 | LCC3b3 |
| 208001 | CD59 | CD59 antigen p18-20 (antigen identified by monoclonal antibodies 16.3A5, EJ16, EJ30, EL32 | LCC2b4 |
| | | and G344) | |
| 208699 | ESTs | ESTs | LCC4f7 |
| 212165 | PRDX2 | peroxiredoxin 2 | LCC2h7 |
| 220376 | ESTs | ESTs | LCC9b8 |
| 221632 | EIF2B2 | eukaryotic translation initiation factor 2B, subunit 2 (beta, 39 kD) | LCC6e1 |
| 223141 | ESTs | ESTs | LCC9d9 |
| 232772 | EST(Metallothionein-1B | ESTs, Highly similar to MT1B_HUMAN METALLOTHIONEIN-1B [H. sapiens] | LCC1c12 |
| 233581 | HIP2 | huntingtin interacting protein 2 | LCC3b10 |
| 234398 | TCCCIA00427 | Homo sapiens clone TCCCIA00427 mRNA sequence | LCC3g11 |
| 236305 | HARS | histidyl-tRNA synthetase | LCC8c12 |
| 239877 | HDAC3 | histone deacetylase 3 | LCC3b9 |
| 244147 | ZFP92 | zinc finger protein homologous to Zfp92 in mouse | LCC3e6 |
| 245547 | KIAA0700 | KIAA0700 protein | LCC6c4 |
| 251753 | ESTs | ESTs | LCC5c9 |
| 257197 | NRBF-2 | nuclear receptor binding factor-2 | LCC4h8 |
| 271478 | MAX-interacting protein | MAX-interacting protein 1 | LCC9b10 |
| 276547 | DNMT1 | DNA (cytosine-5-)-methyltransferase 1 | LCC8b11 |
| 284592 | PRO1659 | PRO1659 protein | LCC4f8 |
| 292213 | PERQ1 | PERQ amino acid rich, with GYF domain 1 | LCC1c7 |
| 295140 | FLJ0330 | hypothetical protein FLJ10330 | LCC4d2 |
| 295410 | ESTs | ESTs | LCC3f6 |
| 296998 | ART4 | ADP-ribosyltransferase 4 | LCC1h9 |
| 298155 | ACADM | acyl-Coenzyme A dehydrogenase, C-4 to C-12 straight chain | LCC2b2 |
| 298965 | COX6B | cytochrome c oxidase subunit VIb | LCC5g11 |
| 307532 | EIF4A2 | eukaryotic translation initiation factor 4A, isoform 2 | LCC8d7 |
| 310493 | FACL3 | fatty-acid-Coenzyme A ligase, long-chain 3 | LCC3c10 |
| 321189 | RAP1B | RAP1B, member of RAS oncogene family | LCC3d12 |
| 321661 | PPP2R5C | protein phosphatase 2, regulatory subunit B (B56), gamma isoform | LCC1b4 |
| 321859 | ESTs | ESTs | LCC4h10 |
| 322759 | SNAPC5 | small nuclear RNA activating complex, polypeptide 5, 19 kD | LCC4b8 |
| 323474 | ARF1 | ADP-ribosylation factor 1 | LCC8d11 |
| 325062 | SLC20A1 | solute carrier family 20 (phosphate transporter), member 1 | LCC1e3 |
| 325102 | EST(CTB2) | ESTs, Moderately similar to CTB2_HUMAN C-TERMINAL BINDING PROTEIN 2□ | LCC3d10 |
| | | [H. sapiens] | |
| 327304 | H326 | H326 | LCC1f9 |
| 340840 | FLJ20263 (AKAP450) | Homo sapiens cDNA FLJ20263 fis, clone COLF7804, highly similar to AJ131693 | LCC3f3 |
| | | Homo sapiens mRNA for AKAP450 protein | |
| 342378 | DUSP5 | dual specificity phosphatase 5 | LCC1d5 |
| 346009 | PFKL | phosphofructokinase, liver | LCC8f9 |
| 358531 | JUN | v-jun avian sarcoma virus 17 oncogene homolog | LCC3b4 |
| 359835 | SAT | spermidine/spermine N1-acetyltransferase | LCC8a5 |
| 359933 | GNAS1 | guanine nucleotide binding protein (G protein), alpha stimulating activity polypeptide 1 | LCC8d9 |
| 361565 | GLUD1 | glutamate dehydrogenase 1 | LCC8a11 |
| 365930 | TAF2F | TATA box binding protein (TBP)-associated factor, RNA polymerase II, F, 55 kD | LCC1e12 |
| 399562 | NUP54 | nucleoporin p54 | LCC6h2 |
| 430318 | PVALB | parvalbumin | LCC2c1 |
| 436051 | ESTs | ESTs, Weakly similar to putative p150 [H. sapiens] | LCC6h10 |
| 449112 | EST(G3PDH) | ESTs, Highly similar to G3P2_HUMAN GLYCERALDEHYDE 3-PHOSPHATE | LCC6h9 |
| | | DEHYDROGENASE, LIVER□ [H. sapiens] | |
| 454970 | DKFZP434G032 | DKFZP434G032 protein | LCC9g12 |
| 469151 | EIF2S2 | eukaryotic translation initiation factor 2, subunit 2 (beta, 38 kD) | LCC8f10 |
| 471863 | DKFZp586C1817 | Homo sapiens mRNA; cDNA DKFZp586C1817 (from clone DKFZp586C1817) | LCC9h9 |
| 509516 | LOC56966 | hypothetical protein from EUROIMAGE 1034327 | LCC5c5 |
| 511521 | CANX | calnexin | LCC2a6 |
| 511586 | HNRPA1 | heterogeneous nuclear ribonucleoprotein A1 | LCC8c11 |
| 564803 | FOXM1 | forkhead box M1 | LCC2h3 |
| 628357 | ACTN3 | actinin, alpha 3 | LCC2a11 |
| 665774 | EIF4E | eukaryotic translation initiation factor 4E | LCC1h7 |
| 711959 | RPC62 | polymerase (RNA) III (DNA directed) (62 kD) | LCC2f12 |
| 712840 | STAT5B | signal transducer and activator of transcription 5B | LCC2d4 |
| 712848 | MADD | MAP-kinase activating death domain | LCC2h4 |
| 713647 | TSPAN-3 | tetraspan 3 | LCC2f6 |
| 714210 | RY1 | putative nucleic acid binding protein RY-1 | LCC3a4 |
| 725274 | TTC1 | tetratricopeptide repeat domain 1 | LCC2d3 |
| 730149 | TCEA2 | transcription elongation factor A (SII), 2 | LCC1d4 |
| 739183 | CD68 | CD68 antigen | LCC3a12 |
| 739625 | KIAA0973 | KIAA0973 protein | LCC2h10 |
| 739993 | BRE | brain and reproductive organ-expressed (TNFRSF1A modulator) | LCC2g11 |
| 740914 | CTBP1 | C-terminal binding protein 1 | LCC2h5 |
| 741067 | SMARCD2 | SWI/SNF related, matrix associated, actin dependent regulator of chromatin, subfamily d, | LCC1f6 |
| | | member 2 | |
| 741988 | ACY1 | aminoacylase 1 | LCC8f6 |
| 745604 | BCAR1 | breast cancer anti-estrogen resistance 1 | LCC8g8 |
| 753313 | LAPTM5 | Lysosomal-associated multispanning membrane protein-5 | LCC1e2 |
| 753457 | NDUFS1 | NADH dehydrogenase (ubiquinone) Fe-S protein 1 (75 kD) (NADH-coenzyme Q reductase) | LCC1b11 |
| 753897 | AMFR | autocrine motility factor receptor | LCC2a10 |
| 755444 | TMSB4X | thymosin, beta 4, X chromosome | LCC6e12 |
| 756490 | BCAT2 | branched chain aminotransferase 2, mitochondrial | LCC5a12 |
| 756600 | PPIB | peptidylprolyl isomerase B (cyclophilin B) | LCC8d8 |
| 756769 | CHAF1B | chromarin assembly factor 1, subunit B (p60) | LCC8b3 |
| 756968 | EFNB1 | ephrin-B1 | LCC2g7 |
| 758365 | OS4 | conserved gene amplified in osteosarcoma | LCC3b7 |
| 758662 | PSMD9 | proteasome (prosome, macropain) 26S subunit, non-ATPase, 9 | LCC2e1 |
| 759200 | DHPS | deoxyhypusine synthase | LCC8e9 |
| 760298 | PRSC1 | protease, cysteine, 1 (legumain) | LCC2e7 |
| 770080 | PXN | paxillin | LCC1d2 |
| 770388 | CLDN4 | claudin 4 | LCC5b1 |
| 773147 | FLJ10491 | Homo sapiens cDNA FLJ10491 fis, clone NT2RP2000239 | LCC5e3 |
| 773367 | COMT | catechol-O-methyltransferase | LCC8f4 |
| 774071 | CLTH | Clathrin assembly lymphoid-myeloid leukemia gene | LCC2g2 |
| 781704 | TRIP7 | thyroid hormone receptor interactor 7 | LCC2g12 |
| 783698 | KIAA0188 | KIAA0188 protein | LCC2e12 |
| 784278 | SF100 | nuclear antigen Sp100 | LCC2c9 |
| 784841 | EIF2S3 | eukaryotic translation initiation factor 2, subunit 3 (gamma, 52 kD) | LCC2b10 |
| 786048 | E2F4 | E2F transcription factor 4, p107/p130-binding | LCC3a3 |
| 788574 | GCN5L2 | GCN5 (general control of amino-acid synthesis, yeast, homolog)-like 2 | LCC2g8 |
| 789232 | PSMD4 | proteasome (prosome, macropain) 26S subunit, non-ATPase, 4 | LCC2g4 |
| 795282 | HSPC126 | HSPC126 protein | LCC4h3 |
| 795330 | NR1D1 | nuclear receptor subfamily 1, group D, member 1 | LCC2b11 |
| 795888 | RBBP2 | retinoblastoma-binding protein 2 | LCC2b12 |
| 809517 | PRO2605 | hypothetical protein PRO2605 | LCC4g7 |
| 809648 | ZNF162 | zinc finger protein 162 | LCC2g10 |
| 809835 | HNRPC | heterogeneous nuclear ribonucleoprotein C (C1/C2) | LCC8d12 |
| 809992 | PSMD2 | proteasome (prosome, macropain) 26S subunit, non-ATPase, 2 | LCC1g9 |
| 809992 | PSMD2 | proteasome (prosome, macropain) 26S subunit, non-ATPase, 2 | LCC8b8 |
| 810019 | HNRPD | heterogeneous nuclear ribonucleoprotein D (AU-rich element RNA-binding protein 1, 37 kD) | LCC8a10 |
| 810791 | MNAT1 | menage a trois 1 (CAK assembly factor) | LCC8b7 |
| 810873 | SCNN1A | sodium channel, nonvoltage-gated 1 alpha | LCC1a8 |
| 811792 | GSS | glutathione synthetase | LCC1h2 |
| 813158 | DRG2 | developmentally regulated GTP-binding protein 2 | LCC1g11 |
| 813280 | ADSL | adenylosuccinate lyase | LCC2a12 |
| 813426 | G53955 | GS3955 protein | LCC1f4 |
| 813648 | DLD | dihydrolipoamide dehydrogenase (E3 component of pyruvate dehydrogenase complex, | LCC8b10 |
| | | 2-oxo-glutarate complex, branched chain keto acid dehydrogenase complex) | |
| 813742 | PTK7 | PTK7 protein tyrosine kinase 7 | LCC1b2 |
| 814508 | PPP1R7 | protein phosphatase 1, regulatory subunit 7 | LCC2h9 |
| 814595 | PRKCBP1 | protein kinase C binding protein 1 | LCC2d5 |
| 814636 | SMARCA2 | SWI/SNF related, matrix associated, actin dependent regulator of chromatin, subfamily a, | LCC2e3 |
| | | member 2 | |
| 815542 | MX1 | myxovirus (influenza) resistance 1, homolog of marine (interferon-inducible protein p78) | LCC2c10 |
| 815575 | ACTR1A | ARP1 (actin-related protein 1, yeast) homolog A (centractin alpha) | LCC8f3 |
| 823930 | ARPC1A | actin related protein 2/3 complex, subunit 1A (41 kD) | LCC1g7 |
| 824024 | NQO2 | NAD(P)H menadione oxidoreductase 2, dioxin-inducible | LCC2c3 |
| 824031 | HSJ2 | heat shock protein, DNAJ-like 2 | LCC3a7 |
| 824602 | IFI16 | interferon, gamma-inducible protein 16 | LCC2f7 |
| 825470 | TOP2A | topoisomerase (DNA) II alpha (170 kD) | LCC2b7 |
| 838366 | HMGCL | 3-hydroxymethyl-3-methylglutaryl-Coenzyme A lyase (hydroxymethylglutaricaciduria) | LCC8g4 |
| 840404 | MGAT2 | mannosyl (alpha-1,6-)-glycoprotein beta-1,2-N-acetylglucosaminyltransferase | LCC2g6 |
| 840940 | PABPC1 | poly(A)-binding protein, cytoplasmic 1 | LCC2c8 |
| 841691 | MNPEP | methionine aminopeptidase; eIF-2-associated p67 | LCC8c9 |
| 843016 | P130 | nucleolar phosphoprotein p130 | LCC2f5 |
| 843328 | DUSP12 | dual specificity phosphatase 12 | LCC5c2 |
| 852520 | UQCRC2 | ubiquinol-cytochrome c reductase core protein II | LCC8e2 |
| 853570 | SLC25A6 | solute carrier family 25 (mitochondrial carrier; adenine nucleotide translocator), member 6 | LCC8f5 |
| 855910 | LGALS3 | lectin, galactoside-binding, soluble, 3 (galectin 3) | LCC5a9 |
| 866882 | FDFT1 | farnesyl-diphosphate farnesyltransferase 1 | LCC8e8 |
| 868368 | TMSB4X | thymosin, beta 4, X chromosome | LCC5a11 |
| 877613 | DCTN1 | dynactin 1 (p150, Glued (Drosophila) homolog) | LCC2h8 |
| 877832 | DXS1357E | accessory proteins BAP31/BAP29 | LCC8e5 |
| 878545 | RPL18 | ribosomal protein L18 | LCC6c9 |
| 884644 | HBG1 | hemoglobin, gamma A | LCC5a10 |
| 897164 | CTNNA1 | catenin (cadherin-associated protein), alpha 1 (102 kD) | LCC8e7 |
| 897177 | PGAM1 | phosphoglycerate mutase 1 (brain) | LCC8e3 |
| 897626 | PRO2706 | hypothetical protein PRO2706 | LCC2h11 |
| 897880 | CCT4 | chaperonin containing TCP1, subunit 4 (delta) | LCC8d6 |
| 897983 | KIAA0106 | anti-oxidant protein 2 (non-selenium glutathione peroxidase, acidic calcium-independent | LCC2f9 |
| | | phospholipase A2) | |
| 898262 | UBE1 | ubiquitin-activating enzyme E1 (A1S9T and BN75 temperature sensitivity complementing) | LCC8c3 |
| 949928 | ZNF220 | zinc finger protein 220 | LCC2e2 |
| 950489 | SOD1 | superoxide dismutase 1, soluble (amyotrophic lateral sclerosis 1 (adult)) | LCC8b12 |
| 950682 | PFKP | phosphofructokinase, platelet | LCC8c5 |
| 951117 | SHMT2 | serine hydroxymethyltransferase 2 (mitochondrial) | LCC3b6 |
| 951313 | GP1 | glucose phosphate isomerase | LCC5c6 |
| 969854 | CALM3 | calmodulin 3 (phosphorylase kinase, delta) | LCC8e4 |
| 971367 | RPS8 | ribosomal protein S8 | LCC6c10 |
| 1160558 | PTS | 6-pyruvoyltetrahydropterin synthase | LCC6c3 |
| 1340595 | HNRPL | heterogeneous nuclear ribonucleoprotein L | LCC6b12 |
| 1416782 | CKB | creatine kinase, brain | LCC8f7 |
| 1473300 | HADHA | hydroxyacyl-Coenzyme A dehydrogenase/3-ketoacyl-Coenzyme A thiolase/enoyl-Coenzyme A | LCC8f11 |
| | | hydratase (trifunctional protein), alpha subunit | |
| 1475028 | RPS27 | ribosomal protein S27 (metallopanstimulin 1) | LCC6c8 |
| 1475730 | CCT6A | chaperonin containing TCP1, subunit 6A (zeta 1) | LCC8f12 |

TABLE 2
Gene expression profiles in breast cancer cell lines

| Gene Name | Clone ID | Plate Pos. | MB231-1A | MB231-1B | MB231-2A | MB231-2B | MB231-1 |
|---|---|---|---|---|---|---|---|
| TNC | 23185 | LCC9d11 | 0.744 | 0.811 | 0.773 | 0.611 | 0.7775 |
| ALDOC | 23831 | LCC1e11 | 0.877 | 0.944 | 1.038 | 1.043 | 0.9105 |
| ALCAM | 26617 | LCC2b1 | 0.563 | 0.595 | 0.582 | 0.526 | 0.579 |
| NCBP2 | 26711 | LCC1g10 | 0.55 | 0.562 | 0.603 | 0.615 | 0.556 |
| LOC57862 | 28098 | LCC1e5 | 0.846 | 0.849 | 0.713 | 0.688 | 0.8475 |
| karyopherin a2 | 28116 | LCC9e1 | 1.167 | 1.23 | 1.169 | 1.204 | 1.1985 |
| ESTs | 30476 | LCC9d8 | 2.033 | 2.229 | 2.179 | 1.895 | 2.131 |
| FLJ10509 | 32517 | LCC8e12 | 1.039 | 1.09 | 1.14 | 1.028 | 1.0645 |
| PRPSAP1 | 33949 | LCC1d8 | 1.38 | 1.575 | 1.347 | 1.296 | 1.4775 |
| Fibronectin 1 | 36191 | LCC9d10 | 0.559 | 0.59 | 0.639 | 0.542 | 0.5745 |
| IMPDH1 | 39884 | LCC8a8 | 0.541 | 0.572 | 0.547 | 0.53 | 0.5565 |
| SLC25A4 | 40026 | LCC8g5 | 1.716 | 1.829 | 1.465 | 1.704 | 1.7725 |
| TEGT | 44178 | LCC9d7 | 1.4 | 1.458 | 1.511 | 1.291 | 1.429 |
| RPML3 | 44255 | LCC8a7 | 0.594 | 0.627 | 0.552 | 0.639 | 0.6105 |
| MAP2K3 | 45641 | LCC3a10 | 0.605 | 0.627 | 0.602 | 0.67 | 0.616 |
| ESTs | 45801 | LCC5b5 | 0.62 | 0.66 | 0.661 | 0.579 | 0.64 |
| AMID | 49496 | LCC9a12 | 1.494 | 1.709 | 1.686 | 1.712 | 1.6015 |
| ARF4L | 49553 | LCC8g6 | 1.379 | 1.483 | 1.443 | 1.321 | 1.431 |
| GRIA2 | 49987 | LCC8e6 | 0.803 | 0.919 | 0.796 | 0.74 | 0.861 |
| ESTs | 51718 | LCC9b3 | 1.88 | 1.979 | 2.247 | 1.845 | 1.9295 |
| RPL10 | 66686 | LCC1a11 | 2.54 | 2.746 | 2.748 | 2.784 | 2.643 |
| PROCR | 71101 | LCC2h2 | 0.905 | 0.765 | 0.77 | 0.769 | 0.835 |
| KIAA0174 | 79710 | LCC2e10 | 0.963 | 1.036 | 1.137 | 1.114 | 0.9995 |
| SLC1A5 | 80910 | LCC2e8 | 3.853 | 4.213 | 3.84 | 4.022 | 4.033 |
| SF3A1 | 108667 | LCC8b6 | 0.574 | 0.585 | 0.695 | 0.498 | 0.5795 |
| ESTs | 112576 | LCC3e1 | 0.684 | 0.75 | 0.973 | 0.699 | 0.717 |
| ESTs | 114101 | LCC9c8 | 0.685 | 0.716 | 0.682 | 0.733 | 0.7005 |
| POH1 | 127519 | LCC1f11 | 1.143 | 1.191 | 1.155 | 1.102 | 1.167 |
| ACP5 | 127821 | LCC2a8 | 0.723 | 0.775 | 0.866 | 0.785 | 0.749 |
| ADK | 128243 | LCC2b5 | 0.506 | 0.801 | 0.854 | 0.803 | 0.6535 |
| EST | 129585 | LCC3d9 | 0.858 | 0.879 | 0.868 | 0.863 | 0.8685 |
| FLJ13443 | 131563 | LCC4a1 | 0.844 | 0.87 | 0.83 | 0.825 | 0.857 |
| FLJ10976 | 134495 | LCC4a10 | 0.851 | 0.878 | 0.947 | 0.896 | 0.8645 |
| GRP58 | 135083 | LCC8c10 | 0.651 | 0.67 | 0.612 | 0.602 | 0.6605 |
| Fibronectin 1 | 136798 | LCC9a5 | 0.548 | 0.574 | 0.559 | 0.557 | 0.561 |
| PTP IVA | 138345 | LCC9a6 | 0.405 | 0.406 | 0.418 | 0.426 | 0.4055 |
| ESTs | 139883 | LCC4b2 | 0.679 | 0.762 | 0.742 | 0.691 | 0.7205 |
| MCT-1 | 142586 | LCC4f6 | 1.36 | 1.416 | 1.389 | 1.488 | 1.388 |
| ESTs | 144926 | LCC3e5 | 0.859 | 0.988 | 0.923 | 1.047 | 0.9235 |
| PTGS2 | 147050 | LCC9d4 | 0.066 | 0.088 | 0.06 | 0.065 | 0.077 |
| ESTs | 147338 | LCC9a7 | 0.67 | 0.737 | 0.71 | 0.769 | 0.7035 |
| MAM | 163097 | LCC9d1 | 0.652 | 0.839 | 0.681 | 0.634 | 0.7455 |
| SFRS3 | 173554 | LCC6d8 | 1.796 | 2.076 | 1.532 | 1.884 | 1.936 |
| TUBB | 191603 | LCC8a4 | 0.892 | 0.96 | 0.871 | 0.951 | 0.926 |
| ESTs | 198871 | LCC9b6 | 0.99 | 1.022 | 1.053 | 0.904 | 1.006 |
| | 201436 | LCC9c4 | 1.138 | 1.28 | 1.219 | 1.231 | 1.209 |
| THBD | 205185 | LCC1a7 | 0.173 | 0.185 | 0.114 | 0.108 | 0.179 |
| SLC2A1 | 207358 | LCC3b3 | 0.593 | 0.592 | 0.649 | 0.696 | 0.5925 |
| CD59 | 208001 | LCC2b4 | 0.818 | 0.914 | 0.832 | 0.898 | 0.866 |
| ESTs | 208699 | LCC4f7 | 0.574 | 0.641 | 0.671 | 0.596 | 0.6075 |
| PRDX2 | 212165 | LCC2h7 | 0.735 | 0.782 | 0.721 | 0.721 | 0.7585 |
| ESTs | 220376 | LCC9b8 | 1.517 | 1.46 | 1.46 | 1.283 | 1.4885 |
| EIF2B2 | 221632 | LCC6e1 | 0.443 | 0.474 | 0.43 | 0.499 | 0.4585 |
| ESTs | 223141 | LCC9d9 | 1.125 | 1.116 | 1.18 | 0.962 | 1.1205 |
| EST(MTT-1B) | 232772 | LCC1c12 | 0.84 | 0.848 | 1.174 | 0.92 | 0.844 |
| HIP2 | 233581 | LCC3b10 | 0.615 | 0.643 | 0.623 | 0.59 | 0.629 |
| TCCCIA00427 | 234398 | LCC3g11 | 1.221 | 1.317 | 1.198 | 1.333 | 1.269 |
| HARS | 236305 | LCC8c12 | 1.073 | 1.131 | 1.239 | 1.175 | 1.102 |
| HDAC3 | 239877 | LCC3b9 | 1.046 | 1.013 | 1.062 | 1.175 | 1.0295 |
| ZFP92 | 244147 | LCC3e6 | 0.829 | 0.865 | 0.784 | 0.89 | 0.847 |
| KIAA0700 | 245547 | LCC6c4 | 0.646 | 0.713 | 0.668 | 0.769 | 0.6795 |
| ESTs | 251753 | LCC5c9 | 0.852 | 0.901 | 0.833 | 0.878 | 0.8765 |
| NRBF-2 | 257197 | LCC4h8 | 0.981 | 0.963 | 0.908 | 0.967 | 0.972 |
| MAX-IP1 | 271478 | LCC9b10 | 0.758 | 0.789 | 0.772 | 0.687 | 0.7735 |
| DNMT1 | 276547 | LCC8b11 | 0.923 | 1.099 | 1.13 | 1.059 | 1.011 |
| PRO1659 | 284592 | LCC4f8 | 1.06 | 1.145 | 1.012 | 1.042 | 1.1025 |
| PERQ1 | 292213 | LCC1c7 | 1.213 | 1.347 | 1.097 | 1.096 | 1.28 |
| FLJ10330 | 295140 | LCC4d2 | 1.248 | 1.565 | 1.34 | 1.17 | 1.4065 |
| ESTs | 295410 | LCC3f6 | 1.362 | 1.364 | 1.409 | 1.355 | 1.363 |
| ART4 | 296998 | LCC1h9 | 0.697 | 0.762 | 0.602 | 0.606 | 0.7295 |
| ACADM | 298155 | LCC2b2 | 1.56 | 1.724 | 1.612 | 1.633 | 1.642 |
| COX6B | 298965 | LCC5g11 | 1.604 | 1.678 | 1.565 | 1.799 | 1.641 |
| EIF4A2 | 307532 | LCC8d7 | 1.494 | 2.159 | 1.418 | 1.37 | 1.8265 |
| FACL3 | 310493 | LCC3c10 | 0.884 | 1.289 | 1.328 | 1.478 | 1.0865 |
| RAP1B | 321189 | LCC3d12 | 1.156 | 1.294 | 1.227 | 1.057 | 1.225 |
| PPP2R5C | 321661 | LCC1b4 | 0.696 | 0.734 | 0.768 | 0.645 | 0.715 |
| ESTs | 321859 | LCC4h10 | 0.471 | 0.535 | 0.569 | 0.503 | 0.503 |
| SNAPC5 | 322759 | LCC4b8 | 0.94 | 0.994 | 0.952 | 0.978 | 0.967 |
| ARF1 | 323474 | LCC8d11 | 1.149 | 1.114 | 1.09 | 0.95 | 1.1315 |
| SLC20A1 | 325062 | LCC1e3 | 1.269 | 1.438 | 1.257 | 1.385 | 1.3535 |
| EST(CTB2) | 325102 | LCC3d10 | 1.258 | 1.287 | 1.149 | 1.201 | 1.2725 |
| H326 | 327304 | LCC1f9 | 0.833 | 1.007 | 0.865 | 0.929 | 0.92 |
| FLJ20263(AKAP450) | 340840 | LCC3f3 | 0.883 | 1.089 | 0.803 | 0.673 | 0.986 |
| DUSP5 | 342378 | LCC1d5 | 0.45 | 0.488 | 0.429 | 0.478 | 0.469 |
| PFKL | 346009 | LCC8f9 | 1.132 | 1.188 | 1.099 | 1.027 | 1.16 |
| JUN | 358531 | LCC3b4 | 0.527 | 0.565 | 0.523 | 0.472 | 0.546 |
| SAT | 359835 | LCC8a5 | 0.469 | 0.517 | 0.467 | 0.515 | 0.493 |
| GNAS1 | 359933 | LCC8d9 | 0.929 | 1.131 | 0.959 | 0.825 | 1.03 |
| GLUD1 | 361565 | LCC8a1 | 1.114 | 1.173 | 1.182 | 1.244 | 1.1435 |
| TAF2F | 365930 | LCC1e12 | 0.888 | 0.918 | 0.941 | 0.992 | 0.903 |
| NUP54 | 399562 | LCC6h2 | 1.101 | 1.05 | 1.225 | 1.062 | 1.0755 |
| PVALB | 430318 | LCC2c1 | 0.866 | 0.676 | 0.796 | 0.81 | 0.771 |
| ESTs | 436051 | LCC6h10 | 0.712 | 0.931 | 0.921 | 0.919 | 0.8215 |
| EST(G3PDH) | 449112 | LCC6h9 | 0.935 | 0.925 | 1.034 | 0.89 | 0.93 |
| DKFZP434G032 | 454970 | LCC9g12 | 0.724 | 0.681 | 0.727 | 0.67 | 0.7025 |
| EIF2S2 | 469151 | LCC8f10 | 1.07 | 1.08 | 1.209 | 0.934 | 1.075 |
| DKFZp586C1817 | 471863 | LCC9h9 | 1.486 | 1.689 | 1.51 | 1.086 | 1.5875 |
| LOC56966 | 509516 | LCC5c5 | 1.035 | 1.219 | 0.908 | 0.93 | 1.127 |
| CANX | 511521 | LCC2a6 | 1.473 | 1.601 | 1.737 | 1.655 | 1.537 |
| HNRPA1 | 511586 | LCC8c11 | 1.492 | 1.527 | 1.535 | 1.566 | 1.5095 |
| FOXM1 | 564803 | LCC2h3 | 0.813 | 0.884 | 0.893 | 0.815 | 0.8485 |
| ACTN3 | 628357 | LCC2a11 | 0.782 | 0.756 | 0.821 | 0.855 | 0.769 |
| EIF4E | 665774 | LCC1h7 | 0.705 | 0.752 | 0.672 | 0.699 | 0.7285 |
| RPC62 | 711959 | LCC2f12 | 1.993 | 2.185 | 1.827 | 2.177 | 2.089 |
| STAT5B | 712840 | LCC2d4 | 0.867 | 0.91 | 0.837 | 0.914 | 0.8885 |
| MADD | 712848 | LCC2h4 | 0.775 | 0.832 | 0.893 | 0.854 | 0.8035 |
| TSPAN-3 | 713647 | LCC2f6 | 0.715 | 0.749 | 0.747 | 0.784 | 0.732 |
| RY1 | 714210 | LCC3a4 | 0.838 | 0.917 | 0.832 | 0.906 | 0.8775 |
| TTC1 | 725274 | LCC2d3 | 0.96 | 0.965 | 0.976 | 0.955 | 0.9625 |
| TCEA2 | 730149 | LCC1d4 | 0.796 | 1.113 | 1.051 | 1.03 | 0.9545 |
| CD68 | 739183 | LCC3a12 | 0.88 | 0.92 | 0.974 | 0.952 | 0.9 |
| KIAA0973 | 739625 | LCC2h10 | 1.495 | 1.761 | 1.496 | 1.612 | 1.628 |
| BRE | 739993 | LCC2g11 | 0.926 | 0.966 | 0.963 | 1.082 | 0.946 |
| CTBP1 | 740914 | LCC2h5 | 1.039 | 1.061 | 1.103 | 1.064 | 1.05 |
| SMARCD2 | 741067 | LCC1f6 | 1.212 | 1.206 | 1.103 | 1.037 | 1.209 |
| ACY1 | 741988 | LCC8f6 | 1.556 | 1.691 | 1.71 | 1.413 | 1.6235 |
| BCAR1 | 745604 | LCC8g8 | 0.698 | 0.78 | 0.729 | 0.718 | 0.739 |
| LAPTM5 | 753313 | LCC1e2 | 0.845 | 0.75 | 0.762 | 0.815 | 0.7975 |
| NDUFS1 | 753457 | LCC1b11 | 0.997 | 1.149 | 1.107 | 1.057 | 1.073 |
| AMFR | 753897 | LCC2a10 | 1.516 | 1.667 | 1.592 | 1.535 | 1.5915 |
| TMSB4X | 755444 | LCC6e12 | 1.373 | 1.534 | 1.192 | 1.265 | 1.4535 |
| BCAT2 | 756490 | LCC5a12 | 1.264 | 1.367 | 1.235 | 1.102 | 1.3155 |
| PPIB | 756600 | LCC8d8 | 1.434 | 1.485 | 1.461 | 1.31 | 1.4595 |
| CHAF1B | 756769 | LCC8b3 | 0.972 | 0.902 | 0.891 | 0.769 | 0.937 |
| EFNB1 | 756068 | LCC2g7 | 1.258 | 1.292 | 1.17 | 1.287 | 1.275 |
| OS4 | 758365 | LCC3b7 | 1.23 | 1.278 | 1.202 | 1.323 | 1.254 |
| PSMD9 | 758662 | LCC2e1 | 0.822 | 0.842 | 0.826 | 0.931 | 0.832 |
| DHPS | 759200 | LCC8e9 | 1.048 | 1.228 | 1.05 | 1.161 | 1.138 |
| PRSC1 | 760298 | LCC2e7 | 0.405 | 0.422 | 0.375 | 0.422 | 0.4135 |
| PXN | 770080 | LCC1d2 | 0.844 | 0.852 | 0.832 | 0.868 | 0.848 |
| CLDN4 | 770388 | LCC5b1 | 5.1407 | 5.7961 | 6.276 | 5.436 | 5.4684 |
| FLJ10491 | 773147 | LCC5e3 | 1.42 | 1.466 | 1.521 | 1.42 | 1.443 |
| COMT | 773367 | LCC8f4 | 1.074 | 1.195 | 1.173 | 0.939 | 1.1345 |
| CLTH | 774071 | LCC2g2 | 0.773 | 0.811 | 0.731 | 0.729 | 0.792 |
| TRIP7 | 781704 | LCC2g12 | 0.846 | 0.937 | 1.027 | 0.992 | 0.8915 |
| KIAA0188 | 783698 | LCC2e12 | 1.417 | 1.489 | 1.562 | 1.435 | 1.453 |
| SP100 | 784278 | LCC2c9 | 0.773 | 0.811 | 0.803 | 0.851 | 0.792 |
| EIF2S3 | 784841 | LCC2b10 | 2.165 | 2.741 | 2.062 | 2.676 | 2.453 |
| E2F4 | 786048 | LCC3a3 | 1.269 | 1.422 | 1.103 | 1.283 | 1.3455 |
| GCN5L2 | 788574 | LCC2g8 | 0.601 | 0.571 | 0.682 | 0.634 | 0.586 |
| PSMD4 | 789232 | LCC2g4 | 0.996 | 1.072 | 1.143 | 1.174 | 1.034 |
| HSPC126 | 795282 | LCC4h3 | 1.172 | 1.343 | 1.487 | 1.315 | 1.2575 |
| NR1D1 | 795330 | LCC2b11 | 0.301 | 0.363 | 0.315 | 0.282 | 0.332 |
| RBBP2 | 795888 | LCC2b12 | 1.416 | 1.68 | 1.423 | 1.535 | 1.548 |
| PRO2605 | 809517 | LCC4g7 | 0.937 | 0.976 | 0.894 | 0.908 | 0.9565 |
| ZNF162 | 809648 | LCC2g10 | 1.518 | 1.725 | 1.648 | 1.797 | 1.6215 |
| HNRPC | 809835 | LCC8d12 | 1.04 | 1.115 | 1.319 | 1.011 | 1.0775 |
| PSMD2 | 809992 | LCC1g9 | 0.564 | 0.581 | 0.634 | 0.587 | 0.5725 |
| PSMD2 | 809992 | LCC8b8 | 0.542 | 0.573 | 0.597 | 0.507 | 0.5575 |
| HNRPD | 810019 | LCC8a10 | 0.665 | 0.705 | 0.73 | 0.684 | 0.685 |
| MNAT1 | 810791 | LCC8b7 | 0.578 | 0.607 | 0.683 | 0.591 | 0.5925 |
| SCNN1A | 810873 | LCC1a8 | 1.945 | 1.789 | 1.741 | 1.749 | 1.867 |
| GSS | 811792 | LCC1h2 | 0.366 | 0.368 | 0.392 | 0.366 | 0.367 |
| DRG2 | 813158 | LCC1g11 | 0.602 | 0.631 | 0.615 | 0.631 | 0.6165 |
| ADSL | 813280 | LCC2a12 | 1.341 | 1.449 | 1.439 | 1.556 | 1.395 |
| GS3955 | 813426 | LCC1f4 | 1.088 | 1.165 | 1.162 | 1.005 | 1.1265 |
| DLD | 813648 | LCC8b10 | 0.703 | 0.762 | 0.78 | 0.599 | 0.7325 |
| PTK7 | 813742 | LCC1b2 | 0.509 | 0.576 | 0.425 | 0.481 | 0.5425 |
| PPP1R7 | 814508 | LCC2h9 | 1.66 | 1.609 | 1.458 | 1.501 | 1.6345 |
| PRKCBP1 | 814595 | LCC2d5 | 0.754 | 0.834 | 0.762 | 0.686 | 0.794 |
| SMARCA2 | 814636 | LCC2e3 | 1.96 | 2.11 | 2.035 | 2.137 | 2.035 |
| MX1 | 815542 | LCC2c10 | 1.237 | 1.526 | 1.168 | 1.083 | 1.3815 |
| ACTR1A | 815575 | LCC8f3 | 1.318 | 1.384 | 1.193 | 1.145 | 1.351 |
| ARPC1A | 823930 | LCC1g7 | 0.876 | 0.88 | 0.902 | 0.818 | 0.878 |
| NQO2 | 824024 | LCC2c3 | 0.92 | 0.984 | 0.952 | 1.186 | 0.952 |
| HSJ2 | 824031 | LCC3a7 | 1.182 | 1.255 | 1.108 | 1.176 | 1.2185 |
| IFI16 | 824602 | LCC2f7 | 0.607 | 0.613 | 0.596 | 0.527 | 0.61 |
| TOP2A | 825470 | LCC2b7 | 0.68 | 0.69 | 0.671 | 0.65 | 0.685 |
| HMGCL | 838366 | LCC8g4 | 0.934 | 0.932 | 0.897 | 0.951 | 0.933 |
| MGAT2 | 840404 | LCC2g6 | 1.537 | 1.567 | 1.492 | 1.521 | 1.552 |
| PABPC1 | 840940 | LCC2c8 | 0.56 | 0.664 | 0.633 | 0.586 | 0.612 |
| MNPEP | 841691 | LCC8c9 | 1.081 | 1.135 | 1.186 | 1.297 | 1.108 |
| P130 | 843016 | LCC2f5 | 1.017 | 1.067 | 1.09 | 0.996 | 1.042 |
| DUSP12 | 843328 | LCC5c2 | 1.156 | 1.218 | 1.183 | 1.208 | 1.187 |
| UQCRC2 | 852520 | LCC8e2 | 1.073 | 1.134 | 1.116 | 1.082 | 1.1035 |
| SLC25A6 | 853570 | LCC8f5 | 1.724 | 2.027 | 1.775 | 1.649 | 1.8735 |
| LGALS3 | 855910 | LCC5a9 | 0.741 | 0.805 | 0.706 | 0.772 | 0.773 |
| FSFT1 | 866882 | LCC8e8 | 0.749 | 0.779 | 0.834 | 0.783 | 0.764 |
| TMSB4X | 868368 | LCC5a11 | 1.407 | 1.484 | 1.239 | 1.237 | 1.4433 |
| DCTN1 | 877613 | LCC2h8 | 0.878 | 0.944 | 0.877 | 0.956 | 0.911 |
| DXS1357E | 877832 | LCC8e5 | 1.342 | 1.426 | 1.156 | 1.283 | 1.384 |
| RPL18 | 878545 | LCC6c9 | 1.91 | 2.026 | 2.03 | 2.26 | 1.968 |
| HBG1 | 884644 | LCC5a10 | 1.237 | 1.444 | 1.376 | 1.44 | 1.3405 |
| CTNNA1 | 897164 | LCC8e7 | 1.061 | 1.128 | 0.948 | 1.098 | 1.0945 |
| PGAM1 | 897177 | LCC8e3 | 0.993 | 1.113 | 0.97 | 1.1 | 1.053 |
| PRO2706 | 897626 | LCC2h11 | 1.06 | 1.123 | 1.119 | 1.013 | 1.0915 |
| CCT4 | 897880 | LCC8d6 | 1.012 | 0.997 | 1.109 | 0.906 | 1.0045 |
| KIAA0106 | 897983 | LCC2f9 | 1.302 | 1.27 | 1.26 | 1.226 | 1.296 |
| UBE1 | 898262 | LCC8c3 | 0.859 | 1.01 | 0.783 | 0.913 | 0.9345 |
| ZNF220 | 949928 | LCC2e2 | 1.124 | 1.114 | 1.246 | 1.285 | 1.119 |
| SOD1 | 950489 | LCC8b12 | 1.219 | 1.255 | 1.434 | 1.124 | 1.237 |
| PFKP | 950682 | LCC8c5 | 1.325 | 1.398 | 1.271 | 1.454 | 1.3615 |
| SHMT2 | 951117 | LCC3b6 | 2.854 | 3.233 | 2.71 | 2.87 | 3.0435 |
| GPI | 951313 | LCC5c6 | 1.078 | 1.16 | 1.134 | 1.186 | 1.119 |
| CALM3 | 969854 | LCC8e4 | 1.075 | 1.153 | 1.031 | 1.042 | 1.114 |
| RPS8 | 971367 | LCC6c10 | 1.691 | 1.806 | 1.775 | 1.977 | 1.7485 |
| PTS | 1160558 | LCC6c3 | 1.199 | 1.259 | 1.163 | 1.337 | 1.229 |
| HNRPL | 1340595 | LCC6b12 | 0.87 | 0.943 | 0.978 | 0.948 | 0.9065 |
| CKB | 1416782 | LCC8f7 | 0.23 | 0.225 | 0.257 | 0.247 | 0.2275 |
| HADHA | 1473300 | LCC8f11 | 1.113 | 1.151 | 1.234 | 1.041 | 1.132 |
| RPS27 | 1475028 | LCC6c8 | 1.125 | 1.225 | 1.17 | 1.208 | 1.175 |
| CCT6A | 1475730 | LCC8f12 | 1.472 | 1.531 | 1.582 | 1.287 | 1.5015 |

| Gene Name | MB231-2 | MelTis | BCTis-1 | BCTis-2 | MB468 | ZR75-1 | BT549 |
|---|---|---|---|---|---|---|---|
| TNC | 0.692 | 3.4165 | 0.322 | 0.401 | 0.083 | 0.019 | 0.225 |
| ALDOC | 1.0405 | 3.5685 | 0.7555 | 2.9265 | 0.19 | 0.8445 | 0.75 |
| ALCAM | 0.554 | 2.2295 | 0.3495 | 1.2215 | 0.258 | 0.394 | 3.323 |
| NCBP2 | 0.609 | 1.118 | 0.498 | 0.7145 | 0.412 | 0.535 | 0.3855 |
| LOC57862 | 0.7005 | 2.5825 | 0.899 | 2.1045 | 0.9105 | 1.349 | 1.0285 |
| karyopherin a2 | 1.1865 | 0.562 | 0.182 | 0.4275 | 1.1675 | 1.706 | 0.737 |
| ESTs | 2.037 | 2.8725 | 0.845 | 2.0715 | 1.45 | 0.577 | 0.634 |
| FLJ10509 | 1.084 | 1.567 | 0.5135 | 1.509 | 1.2675 | 1.106 | 0.9395 |
| PRPSAP1 | 1.3215 | 3.6375 | 1.1135 | 2.146 | 1.6545 | 2.5855 | 0.9365 |
| Fibronectin 1 | 0.5905 | 1.2415 | 0.3365 | 3.1255 | 0.3715 | 0.0335 | 2.6545 |
| IMPDH1 | 0.5385 | 0.8995 | 0.4395 | 1.2965 | 0.331 | 0.5735 | 0.387 |
| SLC25A4 | 1.5845 | 9.0595 | 1.2235 | 2.1625 | 1.1405 | 1.867 | 1.2185 |
| TEGT | 1.401 | 3.427 | 0.884 | 1.7315 | 0.8495 | 1.1535 | 0.6225 |
| RPML3 | 0.5955 | 0.7495 | 0.296 | 0.7785 | 0.289 | 0.637 | 0.462 |
| MAP2K3 | 0.636 | 1.1385 | 0.431 | 0.741 | 0.7065 | 0.906 | 0.591 |
| ESTs | 0.62 | 3.725 | 0.2185 | 0.5475 | 0.438 | 0.1825 | 1.017 |
| AMID | 1.699 | 2.6895 | 1.2005 | 2.6595 | 3.6495 | 5.6195 | 3.925 |
| ARF4L | 1.382 | 8.4535 | 1.1145 | 1.645 | 1.01 | 1.508 | 0.885 |
| GRIA2 | 0.768 | 1.3815 | 1.826 | 0.724 | 0.3935 | 0.8915 | 0.487 |
| ESTs | 2.046 | 3.758 | 1.7975 | 2.428 | 2.9085 | 2.009 | 0.7815 |
| RPL10 | 2.766 | 8.6115 | 0.9505 | 1.3005 | 2.265 | 2.556 | 2.6805 |
| PROCR | 0.7695 | 3.6755 | 0.4425 | 0.6025 | 1.0405 | 0.852 | 0.9815 |
| KIAA0174 | 1.1255 | 1.211 | 0.881 | 1.5945 | 0.631 | 1.416 | 0.583 |
| SLC1A5 | 3.931 | 3.868 | 5.366 | 2.341 | 3.6915 | 2.947 | 2.5335 |
| SF3A1 | 0.5965 | 0.733 | 0.406 | 0.8295 | 0.2405 | 0.635 | 0.4675 |
| ESTs | 0.836 | 0.8115 | 0.387 | 0.6965 | 0.65 | 0.98 | 0.5765 |
| ESTs | 0.7075 | 1.7875 | 1.388 | 0.974 | 1.4545 | 0.329 | 0.992 |
| POH1 | 1.1285 | 2.8295 | 0.441 | 0.937 | 0.9295 | 1.2075 | 0.7855 |
| ACP5 | 0.8255 | 1.307 | 0.4865 | 0.521 | 1.0315 | 0.817 | 0.535 |
| ADK | 0.8285 | 1.209 | 0.3015 | 0.435 | 0.993 | 0.797 | 0.53 |
| EST | 0.8655 | 0.179 | 0.1535 | 0.2685 | 0.639 | 0.0795 | 0.645 |
| FLJ13443 | 0.8275 | 0.5205 | 0.1785 | 0.266 | 0.8715 | 0.1775 | 0.303 |
| FLJ10976 | 0.9215 | 2.0805 | 0.85 | 1.592 | 1.117 | 0.9175 | 0.6435 |
| GRP58 | 0.607 | 0.9855 | 3.1965 | 2.688 | 0.8735 | 0.544 | 1.415 |
| Fibronectin 1 | 0.558 | 6.0425 | 0.8805 | 3.9185 | 0.3745 | 0.0895 | 3.237 |
| PTP IVA | 0.422 | 1.3295 | 0.422 | 1.0725 | 0.594 | 2.0775 | 1.0525 |
| ESTs | 0.7165 | 0.904 | 0.1415 | 0.3345 | 0.367 | 0.115 | 0.5965 |
| MCT-1 | 1.4385 | 1.292 | 0.546 | 1.4685 | 1.293 | 1.1955 | 1.31 |
| ESTs | 0.985 | 1.779 | 0.5135 | 1.1165 | 0.5125 | 0.7995 | 0.7305 |
| PTGS2 | 0.0625 | 0.1975 | 0.024 | 0.0715 | 0.021 | 0.0145 | 0.193 |
| ESTs | 0.7395 | 1.495 | 0.371 | 0.813 | 0.6285 | 0.218 | 0.873 |
| MAM | 0.6575 | 5.7525 | 0.42 | 1.218 | 1.116 | 0.698 | 1.0755 |
| SFRS3 | 1.708 | 3.2255 | 1.4135 | 1.2645 | 1.758 | 2.104 | 1.325 |
| TUBB | 0.911 | 1.3455 | 0.226 | 0.506 | 1.0425 | 0.9855 | 0.9375 |
| ESTs | 0.9785 | 0.986 | 1.0615 | 1.086 | 0.883 | 1.6875 | 1.4255 |
| | 1.225 | 3.9395 | 2.966 | 1.942 | 3.1845 | 1.92 | 1.091 |
| THBD | 0.111 | 2.3235 | 0.3525 | 0.2235 | 0.244 | 0.2945 | 0.1125 |
| SLC2A1 | 0.6725 | 0.3475 | 0.198 | 0.531 | 0.7935 | 1.893 | 0.2185 |
| CD59 | 0.865 | 2.3895 | 0.82 | 1.177 | 0.961 | 0.8495 | 1.922 |
| ESTs | 0.6335 | 1.144 | 0.088 | 0.2355 | 0.3225 | 0.115 | 0.481 |
| PRDX2 | 0.721 | 1.848 | 2.8795 | 2.5795 | 1.1035 | 2.311 | 0.767 |
| ESTs | 1.3715 | 15.3045 | 3.606 | 1.223 | 1.013 | 1.4025 | 2.688 |
| EIF2B2 | 0.4645 | 0.9435 | 0.233 | 0.387 | 0.359 | 0.49 | 0.4835 |
| ESTs | 1.071 | 3.8935 | 0.7155 | 0.9165 | 1.4355 | 0.8805 | 0.906 |
| EST(MTT-1B) | 1.047 | 0.391 | 0.162 | 0.2235 | 0.7335 | 0.084 | 0.6565 |
| HIP2 | 0.6065 | 1.079 | 0.5295 | 0.761 | 0.634 | 0.8495 | 0.545 |
| TCCCIA00427 | 1.2655 | 1.59 | 1.501 | 3.332 | 0.783 | 0.7435 | 0.5815 |
| HARS | 1.207 | 1.187 | 0.634 | 1.1255 | 0.9515 | 1.273 | 1.015 |
| HDAC3 | 1.1185 | 1.8585 | 0.8905 | 1.0185 | 0.6555 | 1.1155 | 0.618 |
| ZFP92 | 0.837 | 1.59 | 0.5205 | 1.409 | 0.559 | 1.411 | 0.572 |
| KIAA0700 | 0.7185 | 1.694 | 0.5715 | 0.666 | 1.2795 | 0.5085 | 0.4635 |
| ESTs | 0.8555 | 0.961 | 0.2585 | 0.756 | 0.7315 | 0.389 | 0.717 |
| NRBF-2 | 0.9375 | 2.587 | 0.2265 | 0.5405 | 0.959 | 0.526 | 0.683 |
| MAX-IP1 | 0.7295 | 3.3305 | 0.597 | 1.407 | 0.577 | 0.762 | 0.366 |
| DNMT1 | 1.0945 | 0.8405 | 2.0275 | 2.1585 | 0.8315 | 0.954 | 1.284 |
| PRO1659 | 1.027 | 3.599 | 0.187 | 0.275 | 0.842 | 0.021 | 1.444 |
| PERQ1 | 1.0965 | 4.796 | 1.0245 | 3.2815 | 1.568 | 1.1255 | 1.252 |
| FLJ10330 | 1.255 | 1.5485 | 0.486 | 0.833 | 1.04 | 1.6045 | 1.177 |
| ESTs | 1.382 | 2.779 | 2.1015 | 2.0485 | 1.25 | 1.8075 | 1.2765 |
| ART4 | 0.604 | 3.014 | 0.7945 | 1.3 | 0.393 | 0.6045 | 0.686 |
| ACADM | 1.6325 | 1.4885 | 0.3485 | 0.8055 | 0.612 | 0.477 | 1.179 |
| COX6B | 1.682 | 6.4865 | 3.065 | 2.678 | 1.67 | 1.9215 | 1.21 |
| EIF4A2 | 1.394 | 6.512 | 0.4635 | 2.4105 | 0.792 | 0.9455 | 0.911 |
| FACL3 | 1.403 | 1.8865 | 0.5105 | 1.947 | 2.4115 | 1 | 1.1605 |
| RAP1B | 1.142 | 0.7935 | 0.3005 | 0.6745 | 0.535 | 1.029 | 1.0795 |
| PPP2R5C | 0.7065 | 1.481 | 0.565 | 1.0015 | 1.1035 | 1.545 | 0.7925 |
| ESTs | 0.536 | 1.083 | 0.442 | 3.847 | 0.349 | 0.2975 | 0.538 |
| SNAPC5 | 0.965 | 3.2825 | 0.449 | 1.2875 | 0.7585 | 1.5055 | 0.3945 |
| ARF1 | 1.02 | 4.0365 | 0.4755 | 1.469 | 0.586 | 1.2805 | 0.985 |
| SLC20A1 | 1.321 | 3.6525 | 0.414 | 1.1745 | 0.278 | 0.818 | 0.5445 |
| EST(CTB2) | 1.175 | 2.5405 | 2.2555 | 2.0525 | 1.285 | 2.805 | 1.4345 |
| H326 | 0.897 | 3.9665 | 1.95 | 1.2305 | 0.819 | 0.6035 | 0.484 |
| FLJ20263(AKAP450) | 0.738 | 7.9685 | 1.9345 | 2.2725 | 0.603 | 0.8755 | 1.638 |
| DUSP5 | 0.4535 | 0.484 | 0.51 | 1.2275 | 0.156 | 0.062 | 0.061 |
| PFKL | 1.063 | 3.658 | 0.286 | 0.419 | 1.109 | 0.743 | 0.74 |
| JUN | 0.4975 | 1.1825 | 0.8815 | 0.365 | 0.6585 | 0.823 | 0.8815 |
| SAT | 0.491 | 0.642 | 0.1795 | 1.3215 | 3.9775 | 0.111 | 0.469 |
| GNAS1 | 0.892 | 3.6785 | 0.6385 | 0.7105 | 0.6795 | 1.2895 | 1.128 |
| GLUD1 | 1.213 | 0.9235 | 2.0105 | 2.4145 | 0.8475 | 0.81 | 1.265 |
| TAF2F | 0.9665 | 1.076 | 0.3775 | 0.575 | 0.443 | 0.998 | 0.5435 |
| NUP54 | 1.1435 | 1.0595 | 0.253 | 0.5955 | 0.93 | 1.2285 | 1.196 |
| PVALB | 0.803 | 3034.015 | 29.026 | 34162.963 | 4.5445 | 2.8805 | 3.5565 |
| ESTs | 0.92 | 0.999 | 0.289 | 0.4215 | 0.414 | 0.854 | 0.685 |
| EST(G3PDH) | 0.962 | 3.451 | 0.259 | 0.421 | 0.3485 | 0.8045 | 0.54 |
| DKFZP434G032 | 0.6985 | 6.512 | 89.957 | 22.3825 | 1263.422 | 1.834 | 5.4505 |
| EIF2S2 | 1.0715 | 0.6975 | 0.2215 | 0.4095 | 1.34 | 0.5935 | 0.661 |
| DKFZp586C1817 | 1.298 | 4.086 | 0.332 | 0.778 | 0.5305 | 0.317 | 1.083 |
| LOC56966 | 0.919 | 3.756 | 2.7275 | 4.3095 | 1.323 | 1.782 | 0.8035 |
| CANX | 1.696 | 0.7885 | 0.5045 | 1.3235 | 1.6535 | 1.408 | 1.1875 |
| HNRPA1 | 1.5505 | 0.478 | 0.589 | 1.0015 | 0.882 | 1.5625 | 1.062 |
| FOXM1 | 0.854 | 2.216 | 0.4285 | 0.5505 | 0.76 | 0.9105 | 1.1685 |
| ACTN3 | 0.838 | 2.366 | 0.6445 | 0.709 | 0.4335 | 0.541 | 0.671 |
| EIF4E | 0.6855 | 1.0055 | 0.1965 | 0.5985 | 0.5445 | 1.118 | 0.568 |
| RPC62 | 2.002 | 3.1745 | 1.7305 | 1.367 | 4.4425 | 2.506 | 1.769 |
| STAT5B | 0.8755 | 3.293 | 1.177 | 0.9845 | 0.6425 | 0.5175 | 1.269 |
| MADD | 0.8735 | 3.8565 | 1.0775 | 2.284 | 0.6735 | 1.2 | 0.606 |
| TSPAN-3 | 0.7655 | 1.165 | 1.198 | 1.5835 | 0.921 | 0.929 | 0.6765 |
| RY1 | 0.869 | 1.419 | 0.557 | 2.081 | 0.9705 | 1.567 | 0.6545 |
| TTC1 | 0.9655 | 4.09 | 1.0065 | 0.7975 | 0.664 | 0.5235 | 0.673 |
| TCEA2 | 1.0405 | 3.398 | 1.9575 | 2.3155 | 1.239 | 0.6775 | 0.8565 |
| CD68 | 0.963 | 2.552 | 0.444 | 1.186 | 0.204 | 0.27 | 0.2555 |
| KIAA0973 | 1.554 | 4.203 | 1.791 | 1.516 | 1.752 | 1.1825 | 1.133 |
| BRE | 1.0225 | 3.466 | 1.002 | 1.482 | 0.8155 | 0.6545 | 0.6075 |
| CTBP1 | 1.0835 | 4.6745 | 3.0685 | 1.571 | 1.2955 | 1.6935 | 1.3785 |
| SMARCD2 | 1.07 | 6.044 | 3.235 | 2.036 | 2.4705 | 1.552 | 1.0145 |
| ACY1 | 1.5615 | 3.971 | 1.0215 | 1.689 | 0.9565 | 0.9915 | 0.654 |
| BCAR1 | 0.7235 | 1.059 | 0.587 | 0.633 | 0.801 | 1.277 | 0.56 |
| LAPTM5 | 0.7885 | 2.657 | 1.379 | 0.8495 | 0.2375 | 0.2335 | 0.229 |
| NDUFS1 | 1.082 | 3.4775 | 0.5085 | 1.333 | 1.171 | 1.4045 | 0.8045 |
| AMFR | 1.5635 | 4.083 | 1.4875 | 3.179 | 1.9635 | 2.4385 | 1.5355 |
| TMSB4X | 1.2285 | 0.476 | 0.165 | 0.2335 | 0.872 | 0.559 | 1.877 |
| BCAT2 | 1.1685 | 0.12 | 0.1615 | 0.2255 | 1.442 | 0.6745 | 1.9365 |
| PPIB | 1.3855 | 1.404 | 2.69 | 1.053 | 0.6775 | 1.278 | 2.1405 |
| CHAF1B | 0.83 | 4.714 | 1.1595 | 3.514 | 2.2375 | 2.71 | 1.473 |
| EFNB1 | 1.2285 | 2.168 | 0.9755 | 1.162 | 1.6805 | 1.826 | 3.9375 |
| OS4 | 1.2625 | 2.4505 | 1.0495 | 2.309 | 1.038 | 1.5465 | 1.335 |
| PSMD9 | 0.8785 | 2.708 | 0.539 | 1.236 | 0.73 | 0.8435 | 0.401 |
| DHPS | 1.1055 | 2.053 | 1.2455 | 1.435 | 1.2985 | 1.235 | 0.924 |
| PRSC1 | 0.3985 | 2.1255 | 0.341 | 1.0805 | 0.522 | 0.499 | 0.9825 |
| PXN | 0.85 | 1.794 | 0.271 | 0.324 | 0.4075 | 0.3385 | 0.321 |
| CLDN4 | 5.856 | 6.512 | 7.0405 | 3211.802 | 56.151 | 30.974 | 1.379 |
| FLJ10491 | 1.4705 | 6.512 | 0.536 | 3.4925 | 1.51 | 1.7855 | 3.243 |
| COMT | 1.056 | 2.4605 | 0.505 | 0.905 | 0.709 | 0.6915 | 0.962 |
| CLTH | 0.73 | 1.967 | 0.4775 | 0.69 | 1.009 | 0.864 | 0.7125 |
| TRIP7 | 1.0095 | 2.8065 | 1.123 | 1.442 | 2.3765 | 1.3995 | 0.797 |
| KIAA0188 | 1.4985 | 1.3005 | 0.601 | 1.195 | 0.5375 | 0.298 | 0.75 |
| SP100 | 0.827 | 3.287 | 0.855 | 1.373 | 0.6015 | 0.5465 | 0.72 |
| EIF2S3 | 2.369 | 14.7625 | 2.0405 | 3.6135 | 4.507 | 3.2215 | 3.625 |
| E2F4 | 1.193 | 2.0005 | 0.7935 | 0.955 | 0.729 | 1.0715 | 0.6285 |
| GCN5L2 | 0.658 | 1.792 | 0.5175 | 0.575 | 0.298 | 0.3075 | 0.4975 |
| PSMD4 | 1.1585 | 5.0575 | 3.592 | 1.7545 | 1.3555 | 1.7 | 1.2155 |
| HSPC126 | 1.401 | 2.18 | 0.63 | 1.098 | 0.836 | 1.199 | 1.346 |
| NR1D1 | 0.2985 | 4.848 | 0.84 | 3.061 | 0.375 | 0.206 | 0.4425 |
| RBBP2 | 1.479 | 6.402 | 2.1615 | 3.8545 | 1.5485 | 1.958 | 2.908 |
| PRO2605 | 0.901 | 1.724 | 0.658 | 1.3111 | 2.235 | 1.0535 | 0.5415 |
| ZNF162 | 1.7225 | 4.0705 | 1.985 | 1.5815 | 1.8715 | 1.17 | 1.0205 |
| HNRPC | 1.165 | 1.2015 | 0.351 | 0.949 | 0.739 | 0.8545 | 0.9 |
| PSMD2 | 0.6105 | 1.1135 | 0.3255 | 0.6645 | 0.313 | 0.5505 | 0.3975 |
| PSMD2 | 0.552 | 0.7245 | 0.332 | 0.6755 | 0.354 | 0.565 | 0.389 |
| HNRPD | 0.707 | 1.3535 | 2.669 | 3.918 | 0.8065 | 0.8555 | 1.2305 |
| MNAT1 | 0.637 | 3.608 | 0.3295 | 0.721 | 0.326 | 0.6645 | 0.419 |
| SCNN1A | 1.745 | 20.372 | 3.2285 | 4.603 | 30.8785 | 1.8085 | 2.378 |
| GSS | 0.379 | 1.2795 | 1.039 | 0.4315 | 0.1675 | 0.2085 | 0.475 |
| DRG2 | 0.623 | 1.545 | 0.4875 | 0.797 | 0.3885 | 0.562 | 0.431 |
| ADSL | 1.4975 | 2.099 | 0.483 | 0.8595 | 0.7255 | 0.7545 | 0.7115 |
| GS3955 | 1.0835 | 11.633 | 3.84 | 3.052 | 1.99 | 0.3595 | 0.907 |
| DLD | 0.6895 | 0.941 | 3.2445 | 3.756 | 0.871 | 1.072 | 1.3605 |
| PTK7 | 0.453 | 4.8115 | 1.469 | 0.784 | 1.893 | 0.6335 | 1.8735 |
| PPP1R7 | 1.4795 | 4.766 | 2.2 | 3.2585 | 2.2935 | 1.287 | 1.175 |
| PRKCBP1 | 0.724 | 3.5355 | 0.701 | 1.0465 | 0.7785 | 1.212 | 1.093 |
| SMARCA2 | 2.086 | 5.0805 | 3.333 | 2.851 | 2.124 | 1.235 | 0.3525 |
| MX1 | 1.1255 | 13.161 | 11.482 | 7.6555 | 11.108 | 1.24 | 38.2705 |
| ACTR1A | 1.169 | 4.803 | 0.9565 | 1.267 | 0.7945 | 0.91 | 0.8815 |
| ARPC1A | 0.86 | 8.3555 | 1.001 | 1.348 | 0.7215 | 0.8435 | 0.616 |
| NQO2 | 1.069 | 0.7825 | 0.1415 | 0.126 | 0.2615 | 0.055 | 0.049 |
| HSJ2 | 1.142 | 2.51 | 0.9115 | 1.985 | 0.7255 | 1.4695 | 1.8275 |
| IFI16 | 0.5615 | 1.5035 | 0.275 | 0.204 | 0.019 | 0.016 | 0.3175 |
| TOP2A | 0.6605 | 0.3715 | 0.181 | 0.4205 | 0.82 | 0.844 | 0.5655 |
| HMGCL | 0.924 | 3.8995 | 1.1475 | 1.1965 | 0.8875 | 0.858 | 0.721 |
| MGAT2 | 1.5065 | 1.1425 | 0.547 | 0.6215 | 0.3465 | 0.611 | 0.993 |
| PABPC1 | 0.6095 | 2.278 | 0.5095 | 0.3195 | 0.469 | 0.2935 | 0.7905 |
| MNPEP | 1.2415 | 0.7725 | 1.6045 | 1.8895 | 0.7735 | 1.037 | 1.3945 |
| P130 | 1.043 | 1.3645 | 0.2815 | 0.681 | 0.625 | 0.6075 | 1.689 |
| DUSP12 | 1.1955 | 1.7505 | 1.0515 | 0.9915 | 2.0275 | 1.0155 | 0.7885 |
| UQCRC2 | 1.099 | 2.431 | 0.5465 | 1.148 | 0.94 | 2.072 | 0.858 |
| SLC25A6 | 1.712 | 12.1205 | 1.356 | 2.082 | 1.161 | 2.0135 | 1.3713 |
| LGALS3 | 0.739 | 5.6375 | 0.461 | 0.1555 | 0.602 | 0.151 | 1.371 |
| FSFT1 | 0.8085 | 0.697 | 0.876 | 0.704 | 0.749 | 0.935 | 1.0365 |
| TMSB4X | 1.238 | 0.1725 | 0.19 | 0.244 | 1.331 | 0.6875 | 2.1755 |
| DCTN1 | 0.9165 | 3.3475 | 0.7175 | 1.7875 | 0.6715 | 0.99 | 0.573 |
| DXS1357E | 1.2195 | 4.5985 | 1.069 | 1.6945 | 0.933 | 1.2605 | 1.012 |
| RPL18 | 2.145 | 6.2765 | 2.634 | 1.9115 | 1.002 | 1.9055 | 1.202 |
| HBG1 | 1.408 | 1.973 | 0.463 | 0.779 | 0.976 | 1.5835 | 0.947 |
| CTNNA1 | 1.023 | 0.8295 | 0.295 | 0.57 | 0.1075 | 0.5995 | 0.521 |
| PGAM1 | 1.035 | 1.873 | 0.657 | 0.7745 | 0.3095 | 0.6495 | 0.927 |
| PRO2706 | 1.066 | 1.6325 | 0.5065 | 1.412 | 0.9995 | 1.1125 | 0.818 |
| CCT4 | 1.0075 | 0.2765 | 0.0505 | 0.4785 | 0.8575 | 1.2675 | 0.393 |
| KIAA0106 | 1.243 | 0.6715 | 0.3915 | 0.845 | 1.1175 | 0.754 | 0.6045 |
| UBE1 | 0.848 | 3.496 | 1.2705 | 3.717 | 2.1655 | 2.7045 | 1.523 |
| ZNF220 | 1.2655 | 2.1565 | 1.451 | 6.1565 | 0.855 | 0.9455 | 1.333 |
| SOD1 | 1.279 | 2.449 | 1.3525 | 2.821 | 2.0195 | 1.3995 | 0.7495 |
| PFKP | 1.3625 | 4.3135 | 0.763 | 0.7515 | 1.092 | 1.319 | 0.9995 |
| SHMT2 | 2.79 | 2.6455 | 0.6015 | 0.4655 | 1.561 | 1.2105 | 0.9965 |
| GPI | 1.16 | 1.4485 | 1.2495 | 1.3795 | 1.229 | 1.0365 | 1.1715 |
| CALM3 | 1.0365 | 3.228 | 0.779 | 1.0685 | 0.8625 | 0.8105 | 0.733 |
| RPS8 | 1.876 | 3.672 | 0.974 | 1.876 | 1.696 | 1.4415 | 1.407 |
| PTS | 1.25 | 1.9595 | 1.036 | 1.4485 | 2.795 | 0.9325 | 0.9975 |
| HNRPL | 0.963 | 1.693 | 1.4435 | 1.281 | 1.511 | 1.8605 | 1.2505 |
| CKB | 0.252 | 3.5425 | 1.5175 | 0.653 | 0.1605 | 1.278 | 0.2115 |
| HADHA | 1.1375 | 3.8725 | 0.326 | 0.424 | 1.185 | 0.687 | 0.747 |
| RPS27 | 1.189 | 5.5065 | 1.9225 | 1.4805 | 1.096 | 1.156 | 0.771 |
| CCT6A | 1.4345 | 1.3275 | 0.6925 | 1.0815 | 3.487 | 1.0275 | 0.9125 |

| Gene Name | MB134 | MB157 | MB436 | MB453 | BT20 | BT474 | BCTis-3 |
|---|---|---|---|---|---|---|---|
| TNC | 0.083 | 0.229 | 0.2915 | 1.322 | 0.0275 | 0.31 | 0.862 |
| ALDOC | 0.6725 | 0.084 | 0.477 | 0.846 | 0.5965 | 0.3565 | 1.0645 |
| ALCAM | 1.2535 | 0.193 | 0.299 | 2.2875 | 0.0715 | 0.607 | 0.248 |
| NCBP2 | 0.823 | 0.3445 | 0.394 | 0.3165 | 0.6855 | 0.709 | 0.9885 |
| LOC57862 | 1.4175 | 1.389 | 0.753 | 1.8475 | 1.212 | 1.746 | 0.953 |
| karyopherin a2 | 0.536 | 1.156 | 0.672 | 1.1825 | 0.913 | 1.327 | 0.9935 |
| ESTs | 5.3745 | 3.409 | 0.5135 | 2.5625 | 1.2645 | 2.3425 | 1.269 |
| FLJ10509 | 1.4435 | 0.6365 | 0.9125 | 1.3555 | 1.6975 | 1.211 | 1.487 |
| PRPSAP1 | 2.1275 | 2.588 | 0.828 | 2.205 | 1.626 | 1.487 | 3.9915 |
| Fibronectin 1 | 0.052 | 7.429 | 0.6615 | 0.047 | 0.2645 | 0.0825 | 14.9675 |
| IMPDH1 | 0.746 | 0.294 | 0.3385 | 0.3705 | 0.7115 | 0.739 | 0.788 |
| SLC25A4 | 1.1555 | 3.7145 | 1.3925 | 1.3215 | 0.5115 | 2.456 | 1.3635 |
| TEGT | 2.5555 | 0.7165 | 0.3945 | 1.3355 | 1.243 | 1.5195 | 1.5285 |
| RPML3 | 0.75 | 0.2245 | 0.3375 | 0.3575 | 0.733 | 0.7445 | 0.7515 |
| MAP2K3 | 0.8695 | 1.1805 | 0.8895 | 0.533 | 1.0785 | 0.7475 | 0.6815 |
| ESTs | 0.205 | 0.711 | 0.376 | 0.1465 | 0.1745 | 0.143 | 0.437 |
| AMID | 4.7245 | 4.1005 | 6.505 | 2.0425 | 6.565 | 3.195 | 1.4385 |
| ARF4L | 0.9695 | 2.686 | 1.1445 | 1.018 | 0.604 | 1.83 | 1.2565 |
| GRIA2 | 1.084 | 0.488 | 0.365 | 1.245 | 0.7055 | 0.7945 | 0.8765 |
| ESTs | 1.709 | 1.3445 | 1.0245 | 1.998 | 2.459 | 3.2003 | 1.6245 |
| RPL10 | 1.3545 | 1.761 | 3.2775 | 5.325 | 1.172 | 1.668 | 2.157 |
| PROCR | 0.6095 | 1.3195 | 0.9655 | 0.6315 | 1.338 | 0.534 | 0.9085 |
| KIAA0174 | 1.2295 | 0.7765 | 0.6725 | 0.502 | 2.6715 | 1.483 | 0.993 |
| SLC1A5 | 6.2575 | 2.794 | 2.0725 | 3.821 | 2.165 | 2.8855 | 1.453 |
| SF3A1 | 0.7375 | 0.293 | 0.3325 | 0.298 | 0.716 | 0.6905 | 0.714 |
| ESTs | 0.53 | 0.0445 | 0.563 | 0.291 | 0.677 | 0.671 | 0.8965 |
| ESTs | 0.98 | 1.7145 | 1.764 | 0.3215 | 0.346 | 0.8265 | 2.1375 |
| POH1 | 1.282 | 0.707 | 1.1035 | 0.821 | 1.447 | 1.0855 | 0.924 |
| ACP5 | 0.5365 | 0.4775 | 1.424 | 0.4095 | 0.7035 | 0.872 | 0.5625 |
| ADK | 0.639 | 0.4465 | 0.3545 | 0.424 | 0.7015 | 0.992 | 0.4065 |
| EST | 0.3 | 0.147 | 0.2525 | 0.2465 | 0.165 | 0.11 | 0.122 |
| FLJ13443 | 0.224 | 0.584 | 0.1705 | 0.653 | 0.2 | 0.098 | 0.344 |
| FLJ10976 | 1.1 | 0.851 | 0.705 | 0.921 | 2.1265 | 1.29 | 2.2155 |
| GRP58 | 2.3025 | 1.113 | 1.101 | 2.3115 | 1.8155 | 0.762 | 1.4895 |
| Fibronectin 1 | 0.211 | 10.624 | 0.691 | 0.114 | 0.2675 | 0.076 | 32.204 |
| PTP IVA | 1.931 | 1.3455 | 0.6125 | 0.8825 | 0.2185 | 1.107 | 1.0365 |
| ESTs | 0.539 | 0.7465 | 0.4645 | 0.1635 | 0.2185 | 0.1845 | 0.817 |
| MCT-1 | 1.5805 | 5.669 | 1.3235 | 0.516 | 1.963 | 1.1625 | 0.958 |
| ESTs | 0.758 | 0.96 | 0.8875 | 0.584 | 0.456 | 0.523 | 0.8465 |
| PTGS2 | 0.044 | 0.0095 | 0.0815 | 0.0495 | 0.0125 | 0.028 | 0.013 |
| ESTs | 0.359 | 1.08 | 0.7935 | 0.175 | 1.193 | 0.5335 | 1.8195 |
| MAM | 0.7305 | 4.4885 | 0.443 | 0.3025 | 0.23 | 0.5795 | 2.241 |
| SFRS3 | 1.511 | 1.7895 | 1.3305 | 1.3975 | 1.632 | 2.5915 | 2.3715 |
| TUBB | 0.4865 | 0.578 | 0.4805 | 0.3135 | 0.5895 | 0.867 | 0.935 |
| ESTs | 1.476 | 0.8335 | 0.935 | 1.194 | 1.0705 | 3.561 | 2.8205 |
| | 1.843 | 1.4565 | 0.554 | 1.357 | 2.8505 | 5.027 | 1.082 |
| THBD | 0.1645 | 0.0075 | 0.151 | 0.3495 | 0.0405 | 0.089 | 0.1725 |
| SLC2A1 | 3.522 | 0.817 | 0.502 | 0.344 | 0.5585 | 0.8135 | 0.855 |
| CD59 | 0.7925 | 2.476 | 0.7755 | 0.734 | 0.735 | 0.8725 | 7.412 |
| ESTs | 0.114 | 1.0425 | 0.3505 | 0.0955 | 0.1055 | 0.158 | 0.7315 |
| PRDX2 | 0.501 | 0.1875 | 1.208 | 1.525 | 0.8105 | 2.3125 | 0.787 |
| ESTs | 3.729 | 0.3425 | 1.6685 | 0.993 | 4.8455 | 5.5175 | 1.9915 |
| EIF2B2 | 0.5015 | 1.21 | 0.295 | 0.364 | 0.2235 | 0.435 | 0.328 |
| ESTs | 0.9505 | 1.981 | 1.319 | 0.246 | 1.01 | 0.764 | 0.9785 |
| EST(MTT-1B) | 0.2805 | 0.296 | 0.3035 | 0.197 | 0.2125 | 0.1045 | 0.1745 |
| HIP2 | 0.8555 | 1.167 | 0.8825 | 0.506 | 0.9325 | 0.7075 | 0.6435 |
| TCCCIA00427 | 1.7045 | 1.3355 | 1.066 | 1.752 | 1.272 | 2.3445 | 1.254 |
| HARS | 0.834 | 1.173 | 1.0685 | 1.2255 | 1.1475 | 1.0605 | 1.3 |
| HDAC3 | 1.1245 | 1.085 | 0.8495 | 0.571 | 0.898 | 1.044 | 0.7 |
| ZFP92 | 0.9415 | 1.1055 | 0.603 | 1.1 | 0.6825 | 5.3125 | 2.365 |
| KIAA0700 | 0.512 | 1.3745 | 0.7875 | 0.3855 | 1.117 | 0.7535 | 0.457 |
| ESTs | 0.9295 | 1.2825 | 0.733 | 0.403 | 0.5775 | 0.5245 | 0.4285 |
| NRBF-2 | 0.7695 | 0.819 | 0.95 | 0.6775 | 0.644 | 0.599 | 0.53 |
| MAX-IP1 | 1.0845 | 0.8425 | 0.397 | 0.4715 | 0.4095 | 0.3235 | 1.5375 |
| DNMT1 | 1.7225 | 0.8565 | 1.044 | 2.0605 | 1.5975 | 0.754 | 1.1775 |
| PRO1659 | 0.099 | 0.921 | 0.0595 | 0.5335 | 0.0485 | 0.07 | 1.159 |
| PERQ1 | 1.5895 | 5.2495 | 1.145 | 1.556 | 1.03 | 3.25 | 2.3 |
| FLJ10330 | 0.7975 | 1.808 | 0.559 | 1.5015 | 1.8595 | 1.081 | 1.4795 |
| ESTs | 1.3085 | 1.2165 | 1.022 | 0.744 | 0.9165 | 1.873 | 1.553 |
| ART4 | 1.066 | 0.4065 | 0.518 | 0.391 | 0.8325 | 0.902 | 1.1305 |
| ACADM | 2.2965 | 0.821 | 2.154 | 0.4005 | 1.266 | 0.8335 | 2.1475 |
| COX6B | 2.76 | 5.2755 | 1.4165 | 1.1105 | 2.1125 | 3.3935 | 2.335 |
| EIF4A2 | 0.64 | 0.002 | 0.3925 | 2.472 | 1.831 | 1.081 | 1.3555 |
| FACL3 | 4.1225 | 3.1055 | 0.893 | 3.682 | 1.4815 | 3.4015 | 1.081 |
| RAP1B | 1.0365 | 0.9895 | 1.3155 | 0.5945 | 0.9845 | 0.7125 | 1.6955 |
| PPP2R5C | 0.949 | 1.0355 | 0.7945 | 0.906 | 1.0525 | 1.3081 | 1.4955 |
| ESTs | 0.7285 | 0.393 | 0.254 | 0.279 | 0.3775 | 0.5245 | 0.3985 |
| SNAPC5 | 1.091 | 0.945 | 1.3685 | 0.5625 | 1.384 | 1.0605 | 0.662 |
| ARF1 | 0.5135 | 0.194 | 0.804 | 1.633 | 1.467 | 0.4765 | 0.8595 |
| SLC20A1 | 0.533 | 0.0015 | 0.851 | 0.5355 | 0.3375 | 0.2725 | 0.489 |
| EST(CTB2) | 3.8435 | 2.0365 | 0.8045 | 2.281 | 0.934 | 1.801 | 3.129 |
| H326 | 1.026 | 0.7975 | 0.616 | 0.5505 | 0.666 | 1.2165 | 1.6595 |
| FLJ20263(AKAP450) | 2.2365 | 2.7495 | 0.735 | 0.67 | 0.8525 | 1.4945 | 2.4845 |
| DUSP5 | 0.218 | 0.096 | 0.1545 | 0.2435 | 0.037 | 0.0425 | 0.304 |
| PFKL | 0.955 | 1.388 | 0.859 | 0.8835 | 0.918 | 0.4175 | 0.447 |
| JUN | 0.1985 | 0.767 | 0.773 | 1.0665 | 0.507 | 0.6835 | 1.2975 |
| SAT | 0.6935 | 0.1755 | 0.3135 | 0.2115 | 0.225 | 0.627 | 1.149 |
| GNAS1 | 0.8 | 1.0245 | 0.9545 | 1.253 | 2.394 | 1.083 | 0.9915 |
| GLUD1 | 1.7325 | 0.777 | 0.947 | 2.33 | 1.265 | 0.795 | 1.0165 |
| TAF2F | 0.602 | 1.6335 | 0.4445 | 0.539 | 0.8575 | 0.8705 | 1.049 |
| NUP54 | 0.8125 | 0.826 | 0.9855 | 1.041 | 1.152 | 1.7865 | 0.837 |
| PVALB | 5.18 | 9.3575 | 2.5555 | 2.3745 | 2.8045 | 6.814 | 4.736 |
| ESTs | 0.4155 | 0.1175 | 0.7225 | 0.4055 | 1.0255 | 0.6755 | 0.5645 |
| EST(G3PDH) | 0.31 | 0.117 | 0.657 | 0.3365 | 0.9385 | 0.5835 | 0.5265 |
| DKFZP434G032 | 4.919 | 9.817 | 1.4585 | 2.224 | 2.4305 | 2.6 | 10279.539 |
| EIF2S2 | 0.9275 | 1.254 | 0.7515 | 0.7625 | 0.847 | 0.293 | 0.373 |
| DKFZp586C1817 | 1.0435 | 0.683 | 0.4245 | 0.145 | 1.73 | 0.443 | 1.008 |
| LOC56966 | 2.044 | 2.3915 | 0.757 | 0.7455 | 2.525 | 2.741 | 1.254 |
| CANX | 2.065 | 2.773 | 2.262 | 1.528 | 1.2645 | 1.365 | 0.4385 |
| HNRPA1 | 0.697 | 0.5545 | 0.882 | 1.1935 | 0.788 | 0.8645 | 0.956 |
| FOXM1 | 0.4075 | 0.2225 | 1.927 | 0.506 | 1.124 | 0.949 | 1.111 |
| ACTN3 | 0.7415 | 0.4775 | 0.5715 | 0.619 | 0.3935 | 0.5585 | 0.9855 |
| EIF4E | 0.7465 | 0.5455 | 0.701 | 1.369 | 0.937 | 0.9685 | 0.553 |
| RPC62 | 1.2155 | 1.8925 | 2.1285 | 0.976 | 2.1585 | 1.261 | 3.415 |
| STAT5B | 0.9355 | 1.9855 | 0.6505 | 0.4215 | 0.5355 | 0.756 | 0.6965 |
| MADD | 0.9985 | 0.829 | 0.4595 | 0.606 | 0.6385 | 0.781 | 1.221 |
| TSPAN-3 | 2.062 | 1.3325 | 0.468 | 0.0535 | 0.278 | 0.1785 | 0.7485 |
| RY1 | 1.142 | 1.3275 | 1.419 | 0.9495 | 0.936 | 0.8085 | 0.9415 |
| TTC1 | 1.0205 | 1.882 | 0.7765 | 0.6485 | 0.4945 | 0.7855 | 0.6875 |
| TCEA2 | 0.9595 | 2.5575 | 0.375 | 0.5555 | 1.7475 | 1.577 | 0.7495 |
| CD68 | 0.246 | 1.02 | 0.4565 | 0.19 | 0.325 | 0.248 | 1.716 |
| KIAA0973 | 2.847 | 1.851 | 1.41 | 1.1345 | 1.404 | 3.31 | 1.978 |
| BRE | 1.341 | 1.467 | 0.736 | 0.62 | 0.737 | 0.878 | 1.261 |
| CTBP1 | 1.6185 | 1.4985 | 2.015 | 1.4565 | 1.1645 | 1.4155 | 2.496 |
| SMARCD2 | 2.005 | 1.2945 | 0.28 | 1.42 | 2.3675 | 4.6585 | 0.3875 |
| ACY1 | 0.622 | 0.526 | 0.4715 | 0.855 | 1.084 | 1.0755 | 0.925 |
| BCAR1 | 1.102 | 1.231 | 0.6615 | 1.2765 | 0.6835 | 0.7295 | 0.7375 |
| LAPTM5 | 0.459 | 0.1235 | 0.2265 | 0.3665 | 0.1855 | 0.146 | 1.895 |
| NDUFS1 | 1.2935 | 1.4475 | 1.171 | 0.83 | 1.1985 | 1.193 | 1.0725 |
| AMFR | 2.039 | 1.5275 | 1.252 | 1.993 | 2.527 | 1.3955 | 1.577 |
| TMSB4X | 0.359 | 1.0855 | 0.86 | 0.1465 | 0.1915 | 1.476 | 1.1765 |
| BCAT2 | 0.467 | 2.32 | 0.816 | 0.1215 | 0.168 | 1.5805 | 1.7565 |
| PPIB | 2.4055 | 2.753 | 1.6725 | 2.5865 | 1.3085 | 0.533 | 1.289 |
| CHAF1B | 2.8405 | 2.8915 | 1.8245 | 1.4385 | 1.4295 | 2.78 | 2.123 |
| EFNB1 | 2.488 | 3.235 | 1.4295 | 0.8675 | 0.6145 | 1.9215 | 2.2775 |
| OS4 | 1.8455 | 0.979 | 0.5125 | 2.106 | 1.167 | 1.5885 | 3.1285 |
| PSMD9 | 0.905 | 0.8305 | 0.851 | 0.5325 | 0.5155 | 0.9055 | 0.988 |
| DHPS | 0.6635 | 1.165 | 0.923 | 0.868 | 0.761 | 1.175 | 0.6 |
| PRSC1 | 0.9015 | 1.2235 | 0.287 | 0.308 | 0.673 | 0.331 | 2.384 |
| PXN | 0.543 | 0.39 | 0.6675 | 0.39 | 0.678 | 0.529 | 0.5175 |
| CLDN4 | 5.72155 | 5.215 | 1.543 | 9.817 | 28.0275 | 136.3065 | 4175.7835 |
| FLJ10491 | 1.4965 | 0.006 | 2.1085 | 0.45 | 2.634 | 2.1025 | 1.371 |
| COMT | 0.5755 | 0.7285 | 0.6055 | 0.683 | 0.7345 | 0.777 | 0.916 |
| CLTH | 0.495 | 0.7515 | 0.9225 | 0.669 | 1.188 | 0.5015 | 0.905 |
| TRIP7 | 2.5205 | 1.942 | 1.8265 | 1.3735 | 0.5195 | 2.927 | 1.4265 |
| KIAA0188 | 0.9945 | 2.7635 | 0.596 | 0.904 | 0.6005 | 0.736 | 1.779 |
| SP100 | 1.148 | 2.4075 | 0.502 | 0.3085 | 0.6435 | 0.291 | 1.857 |
| EIF2S3 | 3.7685 | 4.4755 | 4.35 | 2.4815 | 3.083 | 5.41 | 5.58 |
| E2F4 | 1.0375 | 0.4285 | 0.7435 | 0.7525 | 1.846 | 1.1245 | 1.037 |
| GCN5L2 | 0.627 | 2.0175 | 3.049 | 0.206 | 0.214 | 0.288 | 0.9845 |
| PSMD4 | 1.5415 | 1.667 | 2.1205 | 1.345 | 1.2155 | 1.497 | 2.6205 |
| HSPC126 | 1.463 | 0.493 | 1.4065 | 1.563 | 1.875 | 1.4075 | 1.557 |
| NR1D1 | 0.613 | 0.983 | 0.45 | 0.243 | 0.2725 | 0.1655 | 2.318 |
| RBBP2 | 2.299 | 1.3615 | 4.235 | 2.2035 | 3.2825 | 2.618 | 3.394 |
| PRO2605 | 15.176 | 1.2685 | 0.4715 | 0.6135 | 0.5905 | 1.4295 | 0.666 |
| ZNF162 | 1.9525 | 2.015 | 1.765 | 1.14 | 1.2475 | 3.7065 | 2.343 |
| HNRPC | 0.7345 | 0.553 | 1.094 | 1.247 | 0.9495 | 0.879 | 0.8185 |
| PSMD2 | 0.7405 | 0.2575 | 0.361 | 0.3135 | 0.6915 | 0.6895 | 0.758 |
| PSMD2 | 0.7315 | 0.2665 | 0.311 | 0.3305 | 0.704 | 0.6935 | 0.649 |
| HNRPD | 2.118 | 1.0625 | 1.042 | 2.2275 | 1.763 | 0.8475 | 1.3195 |
| MNAT1 | 0.7715 | 0.2935 | 0.3705 | 0.417 | 0.784 | 0.7525 | 0.7295 |
| SCNN1A | 71.293 | 2.253 | 2.0385 | 1.5535 | 30.312 | 19.2445 | 4.658 |
| GSS | 0.205 | 0.2935 | 0.3145 | 0.3595 | 0.269 | 0.203 | 0.3285 |
| DRG2 | 0.7805 | 0.2655 | 0.421 | 0.3535 | 0.6865 | 0.72 | 1.04 |
| ADSL | 0.9785 | 1.2665 | 0.7895 | 0.6425 | 0.553 | 0.988 | 0.946 |
| GS3955 | 1.6365 | 1.6395 | 1.1165 | 0.62 | 1.1645 | 0.356 | 1.472 |
| DLD | 2.229 | 1.1315 | 1.05 | 2.6815 | 1.805 | 0.965 | 1.273 |
| PTK7 | 0.8635 | 1.899 | 0.368 | 0.542 | 2.02 | 1.1355 | 3.0105 |
| PPP1R7 | 2.95 | 2.09 | 1.1385 | 1.368 | 1.981 | 1.947 | 2.1225 |
| PRKCBP1 | 1.304 | 1.3 | 0.713 | 1.4 | 1.7865 | 1.563 | 1.526 |
| SMARCA2 | 3.287 | 1.0865 | 1.5145 | 1.4175 | 2.0375 | 0.5805 | 3.353 |
| MX1 | 9.4925 | 177.459 | 5.1435 | 3.2365 | 6.8535 | 2.7345 | 17.302 |
| ACTR1A | 0.602 | 0.662 | 1.055 | 0.561 | 0.762 | 0.776 | 0.908 |
| ARPC1A | 1.1655 | 0.5525 | 0.712 | 0.771 | 1.0795 | 0.9775 | 1.057 |
| NQO2 | 0.073 | 0.0275 | 0.145 | 0.064 | 0.139 | 0.031 | 0.6335 |
| HSJ2 | 1.268 | 1.8435 | 1.872 | 1.81 | 0.719 | 1.747 | 2.3705 |
| IFI16 | 0.082 | 2.3755 | 0.631 | 0.0455 | 0.0685 | 0.0295 | 1.175 |
| TOP2A | 0.2155 | 0.1445 | 0.4195 | 0.1865 | 0.61 | 0.6195 | 0.769 |
| HMGCL | 0.648 | 1.1385 | 1.1415 | 0.5735 | 0.839 | 0.9715 | 1.1035 |
| MGAT2 | 0.5085 | 0.629 | 0.3385 | 1.029 | 0.5015 | 0.438 | 0.532 |
| PABPC1 | 0.4085 | 0.957 | 2.0415 | 0.3885 | 0.5955 | 0.33 | 0.69 |
| MNPEP | 1.697 | 0.9545 | 0.9245 | 1.6125 | 1.3275 | 0.757 | 1.0515 |
| P130 | 0.4415 | 0.716 | 0.8015 | 0.451 | 0.267 | 0.667 | 1.3695 |
| DUSP12 | 1.057 | 1.1505 | 1.004 | 0.92 | 1.0685 | 1.9875 | 1.592 |
| UQCRC2 | 3.0075 | 1.551 | 1.834 | 1.178 | 1.097 | 1.759 | 1.0965 |
| SLC25A6 | 1.2885 | 4.3495 | 1.6095 | 1.4065 | 0.472 | 2.906 | 1.522 |
| LGALS3 | 1.6405 | 2.215 | 0.5115 | 0.27 | 0.75 | 0.8815 | 1.233 |
| FSFT1 | 1.2785 | 0.9515 | 0.608 | 1.719 | 0.767 | 0.6835 | 0.7845 |
| TMSB4X | 0.514 | 2.523 | 0.8575 | 0.1475 | 0.1925 | 1.693 | 1.629 |
| DCTN1 | 1.0465 | 0.9455 | 0.494 | 0.467 | 1.0355 | 0.443 | 1.3535 |
| DXS1357E | 0.8015 | 1.354 | 1.111 | 1.005 | 0.8185 | 1.3365 | 0.9385 |
| RPL18 | 0.91 | 0.6875 | 0.914 | 0.859 | 1.5585 | 1.5095 | 1.271 |
| HBG1 | 0.4 | 0.8015 | 0.9945 | 0.7335 | 0.645 | 0.8825 | 0.615 |
| CTNNA1 | 1.4065 | 0.0005 | 0.309 | 0.6765 | 0.795 | 1.2165 | 0.7585 |
| PGAM1 | 0.5585 | 0.507 | 0.4985 | 0.414 | 0.328 | 0.587 | 0.8925 |
| PRO2706 | 2.172 | 1.4275 | 0.7875 | 1.065 | 1.4985 | 1.67 | 1.056 |
| CCT4 | 0.4345 | 0.608 | 0.9985 | 0.4555 | 1.1 | 0.549 | 0.8025 |
| KIAA0106 | 0.8625 | 0.7125 | 0.8905 | 0.51 | 1.583 | 1.272 | 0.868 |
| UBE1 | 2.7625 | 2.7385 | 1.754 | 1.391 | 1.324 | 2.75 | 2.168 |
| ZNF220 | 1.349 | 1.5735 | 0.6875 | 1.0335 | 0.969 | 1.2055 | 2.333 |
| SOD1 | 1.633 | 2.692 | 2.051 | 1.0645 | 2.5225 | 3.224 | 2.2375 |
| PFKP | 1.37 | 1.374 | 0.6555 | 0.7935 | 1.5745 | 1.422 | 1.168 |
| SHMT2 | 1.106 | 1.502 | 0.717 | 1.0935 | 0.6265 | 0.969 | 1.5105 |
| GPI | 1.981 | 0.804 | 0.368 | 1.126 | 1.1215 | 1.0235 | 0.988 |
| CALM3 | 0.5515 | 0.9525 | 0.8255 | 0.621 | 0.7405 | 0.889 | 1.0095 |
| RPS8 | 0.9335 | 2.07 | 2.18 | 1.119 | 0.867 | 1.7025 | 1.418 |
| PTS | 1.498 | 1.5405 | 1.3865 | 0.893 | 1.5505 | 1.363 | 3.1175 |
| HNRPL | 1.26 | 1.499 | 0.771 | 0.9405 | 1.6075 | 2.3 | 1.939 |
| CKB | 1.374 | 0.8095 | 0.1075 | 2.6755 | 0.111 | 0.0265 | 1.051 |
| HADHA | 1.1565 | 1.4865 | 0.8275 | 0.7885 | 0.8945 | 0.358 | 0.615 |
| RPS27 | 0.585 | 1.8435 | 1.2445 | 1.0555 | 0.6795 | 1.972 | 2 |
| CCT6A | 1.2785 | 2.0385 | 1.201 | 1.0285 | 3.4195 | 2.9105 | 1.096 |

The high quality cDNA microarrays were then used to measure expression of 768 arrayed elements in malignant breast cancer cell lines (n=10) and tissue samples (n=3) using the non-tumorigenic cell line MDA/H6 as a common reference. The name and origin of the breast cancer cell lines and tissues are listed in Table 3. Pearson coefficient of correlation was used to compute the matrix of similarities and dissimilarities between samples and genes. The complex matrix relationships between 15 cancer samples and between 202 genes were simplified and visualized by multidimensional scaling and hierarchical dendrogram clustering analyses. First, the expression profiles of 202 genes in two MDA-MB-231 samples were essentially identical (r=0.982) and the expression pattern of the melanoma sample was the most dissimilar to that of MDA-MB-231 (r=0.325), as expected. Secondly, the expression profiles of other 12 breast cancers were distributed between the identity and the dissimilarity and had their own expression patterns, demonstrating the extensive heterogeneous nature of these breast cancer cells. Thirdly, the expression profiles of BT20, BT474 and ZR75-1 cell lines were more similar to each other (r=0.796) than to others.

TABLE 3
Human Cancer Cell Line and Tissue

| | | Original | |
|---|---|---|---|
| Name | Symbol | Clinical Diagnosis | Source |
| MDA-MB-231 | MB231 | Adenocarcinoma | ATCC¹ |
| MDA/H6 | MDA/H6 | Non-tumorigenic | Dr. Negrini³ |
| MDA-MB-134 | MB134 | Carcinoma | ATCC |
| MDA-MB-157 | MB157 | Carcinoma | ATCC |
| MDA-MB-436 | MB436 | Adenocarcinoma | ATCC |
| MDA-MB-453 | MB453 | Carcinoma | ATCC |
| MDA-MB-468 | MB468 | Adenocarcinoma | ATCC |
| BT-20 | BT20 | Adenocarcinoma | ATCC |
| BT-474 | BT474 | Ductal carcinoma | ATCC |
| BT-549 | BT549 | Ductal carcinoma | ATCC |
| ZR75-1 | ZR751 | Ductal carcinoma | ATCC |
| Breast cancer tissue 1 | BCTis-1 | Poorly differentiated | LCC² |
| | | invasive ductal | |
| | | carcinoma | |
| Breast cancer tissue 2 | BCTis-2 | Poorly differentiated | LCC |
| | | infiltrating ductal | |
| | | carcinoma | |
| Breast cancer tissue 3 | BCTis-3 | Poorly differentiated | LCC |
| | | infiltrating carcinoma | |
| Melanoma tissue | MelTis | Metastatic malignant | LCC |
| | | melanoma | |

¹American Type Culture Collection;
²Lombardi Cancer Center Histology Facility;
³Department of Experimental and Diagnostic Medicine, Section of Microbiology, University of Ferrera, Via Luigi Borsari 46, 44100 Ferrara, Italy.

The microarray gene expression analysis revealed 19 genes with high frequent alterations in their expression in human breast cancers. Out of the 19 genes, 9 genes were over-expressed (Table 4) and 10 genes were under-expressed (Table 5) in breast cancers at the frequencies greater than 77% (n=13).

TABLE 4
Over-expressed BCSGs in breast cancer cell line and tissue samples

| Symbol | Locus ID | Nucleotide Seq. | Amino acid Seq. |
|---|---|---|---|
| MX1 | 4599 | SEQ ID NO:1 | SEQ ID NO:20 |
| PVALB | 5816 | SEQ ID NO:2 | SEQ ID NO:21 |
| RBBP2 | 5927 | SEQ ID NO:3 | SEQ ID NO:22 |
| AIF | 84883 | SEQ ID NO:4 | SEQ ID NO:23 |
| CLDN4 | 1364 | SEQ ID NO:5 | SEQ ID NO:24 |
| KRT23 | 25984 | SEQ ID NO:6 | SEQ ID NO:25 |
| SLC1A5 | 6510 | SEQ ID NO:7 | SEQ ID NO:26 |
| EIF2S3 | 1968 | SEQ ID NO:8 | SEQ ID NO:27 |
| SCNN1A | 6337 | SEQ ID NO:9 | SEQ ID NO:28 |

TABLE 5
Under-expressed BCSGs in breast cancer cell line and tissue samples

| Symbol | Locus ID | Nucleotide Seq. | Amino acid Seq. |
|---|---|---|---|
| THBD | 7056 | SEQ ID NO:10 | SEQ ID NO:29 |
| PTGS2 | 5743 | SEQ ID NO:11 | SEQ ID NO:30 |
| GSS | 2937 | SEQ ID NO:12 | SEQ ID NO:31 |
| DUSP5 | 1847 | SEQ ID NO:13 | SEQ ID NO:32 |
| NQO2 | 4835 | SEQ ID NO:14 | SEQ ID NO:33 |
| TNC | 3371 | SEQ ID NO:15 | SEQ ID NO:34 |
| LAPTM5 | 7805 | SEQ ID NO:16 | SEQ ID NO:35 |
| IFI16 | 3428 | SEQ ID NO:17 | SEQ ID NO:36 |
| CD68 | 968 | SEQ ID NO:18 | SEQ ID NO:37 |
| EIF2B2 | 8892 | SEQ ID NO:19 | SEQ ID NO:38 |

Six of the nine over-expressed genes were known to be involved in human cancers. The interferon-inducible protein p78 (MX1) is over-expressed in human prostate cancer cell line LNCaP (Vaarala et al., Lab. Invest., 80:1259-1268, 2000). Parvalbumin (PVALB) is a Ca²⁺ binding protein and was highly expressed in human carcinoma, mouse neuroblastoma and rat glioma (Pfyffer et al., 412:135-144, 1987). The retinoblastoma binding protein 2 (RBBP2) can bind to the tumor suppressor gene RB and reverse RB-mediated suppression of the activity of the E2F transcription factor (Kim et al., Mol. Cell Biol., 14:7256-7264, 1994). The apoptosis inducible factor (AMID) is a flavoprotein that is normally confined to mitochondria and is sufficient to induce apoptosis of isolated nuclei (Susin et al., Nature, 397:441-446, 1999). Claudin 4 (CLDN4) is a member of the family of tight junction proteins and was shown to up-regulated in ovarian cancer (Hough et al., Cancer Res., 60:6281-6287, 2000). An expression of keratin 23 (KRT23) was highly inducible by pro-apoptotic agent sodium butyrate in different pancreatic cancer cells and this induction was blocked by expression of p21 (WAF1/CIP1) antisense RNA (Zhang et al., 30:123-135, 2001). In addition, soluble carrier family 1 member 5 (SLC1A5) is a neutral amino acid transport-like protein and was up-regulated in 12 of the 13 breast cancer cell lines/tissue samples. Eukaryotic translation initiation factor 2B gamma (EIF2S3) and sodium channel nonvoltage-gated 1α (SCNN1A) were up-regulated in 12 and 10 of the 13 breast cancer cell lines/tissue samples, respectively.

Among the under-expressed genes listed in Table 5, thrombomodulin (THBD), a negative regulator of coagulation, was reported to involve in vascular diseases and cancers. (Kim et al., Anticancer Res., 17:2319-2323, 1997; Hosaka et al., Cancer Lett., 161:231-240,2000). Prostaglandin-endoperoxide synthase 2 (PTGS2) was reported to be undetectable in mammary invasive carcinomas and was more likely detected in ductal carcinomas in situ (Soslow et al., Cancer, 89:2637-2645, 2000). PTGS2 was down-regulated in all 13 breast cancer cell lines/tissue samples. Up-regulation of glutathione synthetase (GSS) is known to increase the cellular levels of glutathione that in turn facilitates growth of certain cells (Huang et al., FASEB J, 15:19-21, 2001). The GSS expression was decreased in 12 of 13 breast cancer cell lines/tissue samples, suggesting that the advanced cancer cells do not require high levels of glutathione for their growth. Dual specificity protein tyrosine phosphatase 5 (DUSP5) is inducible by serum stimulation of fibroblasts and by heat shock, and the DUSP5 induction may lead to the deactivation of mitogen- or stress-activated protein kinase 3 (MAPK3) that participates in cell cycle progression (Ishibashi et al., J. Biol. Chem., 269:29897-29902, 1994). NAD(P)H menadione oxidoreductase 2 (NQO2) is expressed in human heart, brain, lung, liver, and skeletal muscle but is not expressed in placenta, implying its decrease in fast growth tissue. The expression of NQO2 is inducible by antioxidants and its role in cancer remains unknown. Interestingly, the expression of the eukaryotic translation initiation factor 2 beta subunit (EIF2B2) was decreased more than 2 fold in 11 of 13 breast cancer cell lines/tissue samples, whereas the gamma subunit (EIF2S3) was up-regulated in all 13 breast cancer cell lines/tissue samples. The discovery that the inverse levels of EIF2B2 and EIF2S3 were associated with breast cancer progression has not been reported before. Hexabrachion (TNC) is an extracellular matrix glycoprotein that modulates cellular organization (Talts et al. J. Cell Sci., 112:1855-1864, 1999) and the TNC expression was down-regulated in 10 of the 13 breast cancer cell lines/tissue samples.

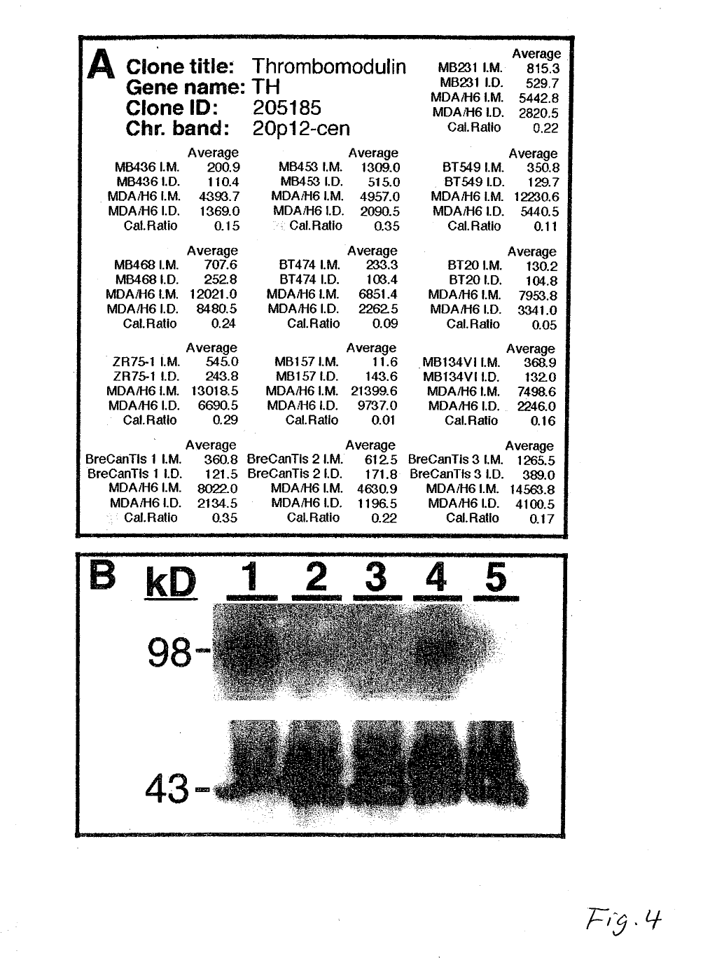

Further analysis demonstrated that the THBD RNA levels decreased in all 13 breast cancer cell lines/tissue samples (FIG. 4, panel (B)). In addition, Western blot analysis correlated the THBD protein expression to its RNA levels in all five cell lines tested. Furthermore, the THBD protein levels were negative in all 18 cases of the advanced breast cancer cells in contrast to normal mammary epithelial cells, measured by in situ immunohistochemical staining (Table 6). It thus appears that THBD expression is inversely correlated to the development of breast cancer.

TABLE 6
Results of in situ immunohistochemical staining of THBD antibody
on 20 cases of breast normal and cancer specimens

| | Pathological diagnosis | | THBD staining | |
|---|---|---|---|---|
| Case | Malignancy | Metastasis to RLN | MEC | BCC |
| 1 | Infiltrating ductal | To 19 of 20 RLN | + + + | − |
| | carcinoma | | | |
| 2 | Infiltrating ductal | To 2 of 2 RLN | + + + | − |
| | carcinoma | | | |
| 3 | Infiltrating lobular and | To 5 of 15 RLN | + + | − |
| | ductal carcinoma | | | |
| 4 | Infiltrating ductal | To 1 of 9 RLN | + + + | − |
| | carcinoma | | | |
| 5 | Infiltrating ductal | To 1 of 12 RLN | + + | − |
| | carcinoma | | | |
| 6 | Infiltrating ductal | NE | + + + | − |
| | carcinoma | | | |
| 7 | Infiltrating carcinoma, | NE | + + + | − |
| | poorly differentiated | | | |
| 8 | Infiltrating ductal | NE | + + + | − |
| | carcinoma | | | |
| 9 | Infiltrating ductal | NE | + + | − |
| | carcinoma | | | |
| 10 | Infiltrating ductal | NE | + + + | − |
| | carcinoma | | | |
| 11 | Infiltrating ductal | NE | + + | − |
| | carcinoma | | | |
| 12 | Infiltrating ductal | NE | + + | − |
| | carcinoma | | | |
| 13 | Infiltrating ductal | NE | + + + | − |
| | carcinoma | | | |
| 14 | Infiltrating ductal | NE | + + + | − |
| | carcinoma | | | |
| 15 | Infiltrating ductal | NE | + + + | − |
| | carcinoma | | | |
| 16 | Infiltrating ductal | NE | + + | − |
| | carcinoma | | | |
| 17 | Infiltrating ductal | NE | + + + | − |
| | carcinoma | | | |
| 18 | Infiltrating ductal | NE | + + | − |
| | carcinoma | | | |
| 19 | Infiltrating | NE | + + + | + + |
| | adenocarcinoma, | | | |
| | moderately well | | | |
| | differentiated | | | |
| 20 | Infiltrating ductal | NE | + + | + + |
| | carcinoma with | | | |
| | intramammary lymphatic | | | |
| | invasion | | | |

Each case of the normal and breast cancer specimens was from the same patients. All the sections were purchased from Lombardi Cancer Center (LCC) Histology Facility. The malignant diagnosis was derived from LCC pathological reports and further verified using HE stained sections. The Metastatic diagnosis was from the LCC pathological reports. The criteria for scoring the positive and negative results are follows: the intensities of immunohistochemical staining: 0 (none), 1 (weak), 2 (moderate), and 3 (strong); distribution of the intensities: 0 (none), 1 (1-15%), 2 (26-50%), 3 (51-75%), and 4 (76-100%); sum=an intensity number+distribution number, sum 0 is score 0, sums 1, 2, and 3 were grouped as score 1, sums 4 and 5 were grouped as score 2, sums 6 and 7 were grouped to score 3; negative (−): means score 0 or score 1, positive (++) means score 2 and positive (+++) means score 3. THBD: thrombomodulin. MEC: normal mammary epithelial cells; BCC: breast cancer cells; RLN: regional lymph nodes. NE: non evidence.

BCSG Products as Markers for Breast Cancer

Most of the BCSGs listed in Tables 4 and 5 have not been previously associated with breast cancer. BCSG homologs from other organisms may also be useful in the use of animal models for the study of breast cancer and for drug evaluation. BCSG homologs from other organisms may be obtained using the techniques outlined below.

In one aspect, the present invention is based on the identification of a number of genes, designated breast-cancer specific genes (BCSGs) set forth in Tables 4 and 5, which are differentially expressed between the breast cancer tissues/cell lines and the non-tumorigenic control tissues/cell lines. The proteins encoded by these genes may in turn be components of disease pathways and thus may serve as markers of breast cancer development or as novel therapeutic targets for treatment and prevention of breast cancer.

Accordingly, the present invention pertains to the use of polynucleotides transcribed from and polypeptides encoded by the BCSGs of Table 4 and 5 as markers for breast cancer. Moreover, the use of expression profiles of these genes can indicate the presence of or a risk of breast cancer. These markers are further useful to correlate differences in levels of expression with a poor or favorable prognosis of breast cancer. For example, panels of the BCSGs can be conveniently arrayed on solid supports for use in kits. The BCSGs can be used to assess the efficacy of a treatment or therapy of breast cancer, or as a target for a treatment or therapeutic agent. The BCSGs can also be used to generate gene therapy vectors that inhibit breast cancer.

Therefore, without limitation as to mechanism, the invention is based in part on the principle that modulation of the expression of the BCSGs of the invention may ameliorate breast cancer, when they are expressed at levels similar or substantially similar to normal (non-diseased) tissue.

As an example, the expression of THBD, one of the BCSGs listed in Table 5, is dowregulated in the parental metastatic breast cancer cell line MDA-MB-231 comparing to the non-tumorigenic derivative MDA/H6. Accordingly, modulation of the down-regulated THBD gene to normal levels (e.g., levels similar or substantially similar to tissue substantially free of breast cancer) may allow for amelioration of breast cancer.

In another embodiment of the invention, a BCSG product (including polynucleotides transcribed from a BCSG and polypeptide translated from such polynucleotides) can be used as a therapeutic compound of the invention. In yet other embodiments, a modulator of an BCSG product of the invention may be used as a therapeutic compound of the invention, or may be used in combination with one or more other therapeutic compositions of the invention. Formulation of such compounds into pharmaceutical compositions is described in subsections below.

In another aspect of the invention, the levels of BCSMs are determined in a particular subject sample for which either diagnosis or prognosis information is desired. The level of a number of BCSMs simultaneously provides an expression profile, which is essentially a “fingerprint” of the presence or activity of a BCSG or plurality of BCSGs that is unique to the state of the cell. In certain embodiments, comparison of relative levels of expression is indicative of the severity of breast cancer, and as such permits for diagnostic and prognostic analysis. Moreover, by comparing relative expression profiles of BCSGs from tissue samples taken at different points in time, e.g., pre- and post-therapy and/or at different time points within a course of therapy, information regarding which genes are important in each of these stages is obtained. The identification of genes that are abnormally expressed in breast cancer tissue versus normal tissue, as well as differentially expressed genes during breast cancer development, allows the use of this invention in a number of ways. For example, comparison of expression profiles of BCSGs at different stages of the disease progression provides a method for long-term prognosing, including survival.

The discovery of the differential gene expression patterns for individual or panels of BCSMs allows for screening of test compounds with the goal of modulating a particular expression pattern. For example, screening can be done for compounds that will convert an expression profile for a poor prognosis to one for a better prognosis. In certain embodiments, this may be done by making biochips comprising sets of BCSMs, which can then be used in these screens. These methods can also be done on the protein level. For example, protein expression levels of the BCSGs can be evaluated for diagnostic and prognostic purposes or to screen test compounds. Furthermore, the modulation of the activity or expression of a BCSM may be correlated with the diagnosis or prognosis of breast cancer.

BCSG-Related Polynucleotides

BCSG-related polynucleotides can be prepared using any of a variety of techniques. For example, a polynucleotide may be identified, as described in more detail below, by screening a microarray of cDNAs for tumor-associated expression (i.e., expression that is at least two fold greater in a breast tumor than in normal tissue, as described in the present invention. Alternatively, polynucleotides may be amplified from cDNA prepared from cells expressing the proteins described herein, such as breast cancer cells. Such polynucleotides may be amplified via polymerase chain reaction (PCR). For this approach, sequence-specific primers may be designed based on the sequences provided herein, and may be purchased or synthesized.

An amplified portion may be used to isolate a full length gene from a suitable library (e.g., a breast cancer cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR, which uses restriction enzymes to generate a fragment in the known region of the gene. The fragment is then circularized by intramolecular ligation and used as a template for PCR with divergent primers derived from the known region. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR and walking PCR. Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., BLAST searches), and such ESTs may be used to generate a contiguous full length sequence. Full length DNA sequences may also be obtained by analysis of genomic fragments.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis. Alternatively, RNA molecules may be generated by in vitro or in vivo transcription of DNA sequences encoding a breast tumor protein, or portion thereof, provided that the DNA is incorporated into a vector with a suitable RNA polymerase promoter (such as T7 or SP6). Certain portions may be used to prepare an encoded polypeptide, as described herein. In addition a portion may be administered to a patient such that the encoded polypeptide is generated in vivo (e.g., by transfecting antigen-presenting cells, such as dendritic cells, with a cDNA construct encoding a breast tumor polypeptide, and administering the transfected cells to the patient).

A portion of a sequence complementary to a coding sequence (i.e., an antisense polynucleotide) may also be used as a probe or to modulate gene expression. cDNA constructs that can be transcribed into antisense RNA may also be introduced into cells or tissues to facilitate the production of antisense RNA. An antisense polynucleotide may be used, as described herein, to inhibit expression of a BCSG protein. Antisense technology can be used to control gene expression through triple-helix formation, which compromises the ability of the double helix to open sufficiently for the binding of polymerases, transcription factors or regulatory molecules. Alternatively, an antisense molecule may be designed to hybridize with a control region of a gene (e.g., promoter, enhancer or transcription initiation site), and block transcription of the gene; or to block translation by inhibiting binding of a transcript to ribosomes.

A portion of a coding sequence, or of a complementary sequence, may also be designed as a probe or primer to detect gene expression. Probes may be labeled with a variety of reporter groups, such as radionuclides and enzymes, and are preferably at least 10 nucleotides in length, more preferably at least 20 nucleotides in length and still more preferably at least 30 nucleotides in length. Primers, as noted above, are preferably 22-30 nucleotides in length.

Any polynucleotide may be further modified to increase stability in vivo. Possible modifications include, but are not limited to, the addition of flanking sequences at the 5′ and/or 3′ ends; the use of phosphorothioate or 2-O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). The polynucleotides may also be administered as naked plasmid vectors. Techniques for incorporating DNA into such vectors are well known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

BCSG-Related Polypeptides

Within the context of the present invention, BCSG-related polypeptides comprise at least a biologically active portion or an immunogenic portion of a BCSG encoded polypeptide or a variant thereof.

Immunogenic portions may generally be identified using well known techniques. Such techniques include screening polypeptides for the ability to react with antigen-specific antibodies, antisera and/or T-cell lines or clones. As used herein, antisera and antibodies are “antigen-specific” if they show immunospecific binding to an antigen (i.e., binding to the antigen with an affinity that is at least 10⁵M⁻¹). Such antisera and antibodies may be prepared as described herein, and using well known techniques. An immunogenic portion of a native breast cancer protein is a portion that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

BCSG related polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

BCSG related polypeptides may be prepared using any of a variety of well known techniques. Recombinant polypeptides encoded by polynucleotides as described above may be readily prepared from the polynucleotides using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and a fusion partner. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Antibodies

The present invention further provides antibodies and antigen-binding fragments thereof, that specifically bind to a BCSM (BCSM-specific antibodies). As used herein, an antibody, or antigen-binding fragment thereof, is said to “specifically bind” to a BCSM if it binds to an antigen with an affinity that is at least 10⁵M⁻¹. As used herein, “binding” refers to a noncovalent association between two separate molecules such that a complex is formed.

Antibodies may be prepared by any of a variety of techniques known to those of ordinary skill in the art. See, e.g., Harlow and Lane, Antibodies. A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using methods well known in the art. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, breasties of hybrids are observed. Single breasties are selected and their culture supernatants tested for binding activity against the polypeptide. Hybridomas having high reactivity and specificity are preferred.

Monoclonal antibodies may be isolated from the supernatants of growing hybridoma breasties. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns and digested by papain to yield Fab and Fe fragments. The Fab and Fc fragments may be separated by affinity chromatography on protein A bead columns.

Additionally, recombinant anti-BCSM antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Humanized antibodies are particularly desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody will preferably also comprise at least a portion of an immunoglobulin constant region (Fe), typically that of a human immunoglobulin.

A therapeutic agent may be coupled (e.g., covalently bonded) to a suitable antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used.

Vectors

Another aspect of the invention pertains to vectors containing a polynucleotide encoding a BCSG protein, or a portion thereof. One type of vector is a “plasmid,” which includes a circular double stranded DNA loop into which additional DNA segments can be ligated. Vectors include expression vectors and gene delivery vectors.

The expression vectors of the invention comprise a polynucleotide encoding a BCSG protein or a portion thereof in a form suitable for expression of the polynucleotide in a host cell, which means that the expression vectors include one or more regulatory sequences, selected on the basis of the host cells to be used for expression, which is operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., BCSG polypeptides, variants of BCSG polypeptides, fusion proteins, and the like).

The expression vectors of the invention can be designed for expression of BCSG polypeptides in prokaryotic or eukaryotic cells. For example, BCSG polypeptides can be expressed in bacterial cells such as E. coli, insect cells (using baculovirus expression vectors) yeast cells or mammalian cells. In certain embodiments, such protein may be used, for example, as a therapeutic protein of the invention. Alternatively, the expression vector can be transcribed and translated in vitro, for example using T7 promoter regulatory sequences and T7 polymerase.

In another embodiment, the expression vector is a yeast expression vector. Examples of vectors for expression in yeast S. cerevisiae include pYepSec1, pMFa, pJRY88, pYES2 (Invitrogen Corporation, San Diego, Calif.), and picZ (Invitrogen Corp, San Diego, Calif.).

Alternatively, BCSG polypeptides of the invention can be expressed in insect cells using baculovirus expression vectors. Baculovirus vectors available for expression of proteins in cultured insect cells (e.g., Sf9 cells) include the pAc series and the pVL series.

In yet another embodiment, a BCSG is expressed in mammalian cells using a mammalian expression vector. When used in mammalian cells, the expression vector's control functions are often provided by viral regulatory elements. For example, commonly used promoters are derived from polyoma, adenovirus 2, cytomegalovirus and Simian Virus 40.

In another embodiment, the mammalian expression vector is capable of directing expression of the polynucleotide preferentially in a particular cell type (e.g., tissue-specific regulatory elements are used to express the polynucleotide). Tissue-specific regulatory elements are known in the art and may include epithelial cell-specific promoters. Other non-limiting examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the marine box promoters and the α-fetoprotein promoter. In certain preferred embodiments of the invention, the tissue-specific promoter is an epithelial cell-specific promoter.

The invention provides a recombinant expression vector comprising a polynucleotide encoding a BCSG cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a BCSG of the invention. Regulatory sequences operatively linked to a polynucleotide cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region, the activity of which can be determined by the cell type into which the vector is introduced.

The invention further provides gene delivery vectors for delivery of polynucleotides to cells, tissue, or to a the mammal for expression. For example, a polynucleotide sequence of the invention can be administered either locally or systemically in a gene delivery vector. These constructs can utilize viral or non-viral vector approaches in in vivo or ex vivo modality. Expression of such coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle is preferably a viral vector and, more preferably, a retroviral, lentiviral, adenoviral, adeno-associated viral (AAV), herpes viral, or alphavirus vectors. The viral vector can also be an astrovirus, coronavirus, orthomyxovirus, papovavirus, paramyxovirus, parvovirus, picornavirus, poxvirus, togavirus viral vector.

Delivery of the gene therapy constructs of this invention into cells is not limited to the above mentioned viral vectors. Other delivery methods and media may be employed such as, for example, liposomes, polycationic condensed DNA linked or unlinked to inactivated adenovirus, ligand linked DNA, naked DNA and eucaryotic cell delivery vehicles cells.

Another aspect of the invention pertains to the expression of BCSGs using a regulatable expression system. Systems to regulate expression of therapeutic genes have been developed and incorporated into the current viral and nonviral gene delivery vectors. Examples of regulatable systems include: the tet-on/off system, the ecdysone system, the progesterone-system, and the rapamycin system.

Methods for Detecting Breast Cancer

In general, breast cancer may be detected in a patient based on the presence of one or more BCSG products (polynucleotides or polypeptide) in a biological sample (for example, blood, sera, sputum urine and/or tumor biopsies) obtained from the patient. In other words, such BCSG products may be used as markers to indicate the presence or absence of breast cancer. In addition, such products may be useful for the detection of other cancers. The antibodies provided herein generally permit detection of the level of antigen that binds to the agent in the biological sample. Polynucleotide primers and probes may be used to detect the levels of transcribed polynucleotides from BCSGs, which is also indicative of the presence or absence of a cancer.

There are a variety of assay formats known to those of ordinary skill in the art for using an antibody to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with an antibody; (b) detecting in the sample a level of polypeptide that binds to the antibody; and (c) comparing the level of polypeptide with a predetermined control value.

In a preferred embodiment, the assay involves the use of antibody immobilized on a solid support to bind to and remove the polypeptide from the remainder of the sample. The bound polypeptide may then be detected using a detection reagent that contains a reporter group and specifically binds to the antibody/polypeptide complex. Such detection reagents may comprise, for example, an antibody that specifically binds to the polypeptide or an antibody or other agent that specifically binds to the antibody, such as an anti-immunoglobulin, protein G, protein A or a lectin. Alternatively, a competitive assay may be utilized, in which a polypeptide is labeled with a reporter group and allowed to bind to the immobilized antibody after incubation of the antibody with the sample. The extent to which components of the sample inhibit the binding of the labeled polypeptide to the antibody is indicative of the reactivity of the sample with the immobilized antibody. Suitable polypeptides for use within such assays include full length breast tumor proteins and portions thereof to which the antibody binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The antibody may be immobilized on the solid support using a variety of techniques known to those of skill in the art. In the context of the present invention, the term “immobilization” refers to both noncovalent association, such as adsorption, and covalent attachment (which may be a direct linkage between the antibody and functional groups on the support or may be a linkage by way of a cross-linking agent). Immobilization by adsorption to a well in a microtiter plate or to a membrane is preferred. In such cases, adsorption may be achieved by contacting the antibody, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of antibody ranging from about 10 ng to about 10 μg, and preferably about 100 ng to about 1 μg, is sufficient to immobilize an adequate amount of the antibody.

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

To determine the presence or absence of breast cancer, the signal detected from the reporter group that remains bound to the solid support is generally compared to a signal that corresponds to a predetermined control value. In one preferred embodiment, the control value for the detection of breast cancer is the average mean signal obtained when the immobilized antibody is incubated with samples from patients without the cancer. A sample generating a signal that is significantly higher (e.g., ≧200%) or lower (e.g., ≦50%) than the control value determined by this method may be considered indicative of cancer.

In a related embodiment, the assay is performed in a flow-through or strip test format, wherein the antibody is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Numerous other assay protocols exist that are suitable for use with the BCSG products or antibodies of the present invention. The above descriptions are intended to be exemplary only. For example, it will be apparent to those of ordinary skill in the art that the above protocols may be readily modified to use BCSG polypeptides to detect antibodies that bind to such polypeptides in a biological sample. The detection of such BCSG-specific antibodies may correlate with the presence of breast cancer.

As noted above, breast cancer may also, or alternatively, be detected based on the level of mRNA transcribed from a BCSG in a biological sample. For example, at least two oligonucleotide primers may be employed in a polymerase chain reaction (PCR) based assay to amplify a portion of a breast tumor cDNA derived from a biological sample, wherein at least one of the oligonucleotide primers is specific for (i.e., hybridizes to) a polynucleotide encoding the breast tumor protein. The amplified cDNA is then separated and detected using techniques well known in the art, such as gel electrophoresis. Similarly, oligonucleotide probes that specifically hybridize to a polynucleotide encoding a breast tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 70%, preferably at least about 80% and more preferably at least about 90%, identity to a portion of a polynucleotide encoding a breast tumor protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence recited in SEQ ID NOS:1-19. Techniques for both PCR based assays and hybridization assays are well known in the art.

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase/decrease in expression in several dilutions of the test patient sample as compared to the same dilutions of the non-cancerous sample may be considered indicative of cancer.

As noted above, to improve sensitivity, multiple BCSG markers may be assayed within a given sample. It will be apparent that antibodies specific for different proteins provided herein may be combined within a single assay. Further, multiple primers or probes may be used concurrently. The selection of BCSG markers may be based on routine experiments to determine combinations that results in optimal sensitivity. In addition, or alternatively, assays for BCSG products provided herein may be combined with assays for other known tumor antigens.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a polypeptide. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may contain at least one oligonucleotide probe or primer, as described above, that hybridizes to a polynucleotide transcribed from a BCSG. Such an oligonucleotide may be used, for example, within a PCR or hybridization assay. Additional components that may be present within such kits include a second oligonucleotide and/or a diagnostic reagent or container to facilitate the detection of a polynucleotide transcribed from a BCSG.

Arrays and Biochips

The invention also includes an array comprising a panel of BCSMs of the present invention. The array can be used to assay expression of one or more genes in the array.

It will be appreciated by one skilled in the art that the panels of BCSMs of the invention may conveniently be provided on solid supports, as a biochip. For example, polynucleotides may be coupled to an array (e.g., a biochip using GeneChip® for hybridization analysis), to a resin (e.g., a resin which can be packed into a column for column chromatography), or a matrix (e.g., a nitrocellulose matrix for northern blot analysis). The immobilization of molecules complementary to the BCSG(s), either covalently or noncovalently, permits a discrete analysis of the presence or activity of each BCSG in a sample. In an array, for example, polynucleotides complementary to each member of a panel of BCSGs may individually be attached to different, known locations on the array. The array may be hybridized with, for example, polynucleotides extracted from a blood or colon sample from a subject. The hybridization of polynucleotides from the sample with the array at any location on the array can be detected, and thus the presence or quantity of the BCSG and BCSG transcripts in the sample can be ascertained. In a preferred embodiment, an array based on a biochip is employed. Similarly, Western analyses may be performed on immobilized antibodies specific for BCSMs hybridized to a protein sample from a subject.

It will also be apparent to one skilled in the art that the entire BCSM (protein or polynucleotide) molecule need not be conjugated to the biochip support; a portion of the BCSM or sufficient length for detection purposes (i.e., for hybridization), for example a portion of the BCSM which is 7, 10, 15, 20, 25, 30, 35, 40, 45, 50, 55, 60, 65, 70, 75, 100 or more nucleotides or amino acids in length may be sufficient for detection purposes.

Identifying Modulators of BCSM

The invention also provides methods for identifying modulators, i.e., candidate agents which (a) bind to a BCSM, or (b) have a modulatory (e.g., stimulatory or inhibitory) effect on the activity of a BCSM or, more specifically, (c) have a modulatory effect on the interactions of the BCSM with one or more of its natural substrates (e.g., peptide, protein, hormone, co-factor, or polynucleotide), or (d) have a modulatory effect on the expression of the BCSMs. Such assays typically comprise a reaction between the BCSM and one or more assay components. The other components may be either the candidate agents itself, or a combination of candidate agents and a binding partner of the BCSM.

The candidate agents of the present invention are generally either small molecules or bioactive agents. In one embodiment the test compound is a small molecule. In another embodiment, the test compound is a bioactive agent. Bioactive agents include but are not limited to naturally-occurring or synthetic compounds or biomolecules. One skilled in the art will appreciate that the nature of the candidate agents may vary depending on the nature of the protein encoded by the BCSG of the invention. For example, if the BCSG encodes an orphan receptor having an unknown ligand, the test compound may be any of a number of bioactive agents which may act as cognate ligand, including but not limited to, cytokines, lipid-derived mediators, small biogenic amines, hormones, neuropeptides, or proteases. In another embodiment, the candidate agents can be an antisense polynucleotide molecule which is complementary to a BCSG polynucleotides.

As used herein, the term “binding partner” refers to a bioactive agent which serves as either a substrate for a BCSM, or alternatively, as a ligand having binding affinity to the BCSM.

Modulators of BCSG expression, activity or binding ability are useful as thereapeutic compositions of the invention. Such modulators (e.g., antagonists or agonists) may be formulated as pharmaceutical compositions, as described herein below. Such modulators may also be used in the methods of the invention, for example, to diagnose, treat, or prognose breast cancer.

Vaccines

Within certain aspects, BCSG products (polypeptides and polynucleotides) described herein may be used as vaccines for breast cancer. Vaccines may comprise one or more such products and an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes. Vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

A vaccine may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be naked DNA. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadellci pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable micro spheres; monophosphoryl lipid A and quil A. Cytokines, such as GM-CSF or interleukin-2, -7, or -12, may also be used as adjuvants.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid. The amount of active compound contained within a sustained release formulation depends upon the site of implantation, the rate and expected duration of release and the nature of the condition to be treated or prevented.

Pharmaceutical Compositions

The invention is further directed to pharmaceutical compositions comprising a pharmaceutically acceptable carrier and at least one of the following: a BCSM, a variant of a BCSM, a BCSM modulator, a BCSM-specific antibody, a vaccine generated using a BCSM or its variant, and a vector capable of expressing a BCSM or a variant of a BCSM.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a BCSM of the invention. Such methods comprise formulating a pharmaceutically acceptable carrier with an agent which modulates expression or activity of a BCSM . Such compositions can further include additional active agents. Thus, the invention further includes methods for preparing a pharmaceutical composition by formulating a pharmaceutically acceptable carrier with an agent which modulates expression or activity of a BCSM and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a BCSM or an anti-BCSM antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The BCSGs of the invention can be inserted into gene delivery vectors and used as gene therapy vectors. Gene therapy vectors can be delivered to a subject by intravascular, intrameucular, subcutaneous, intraperitoneal injection, by direct injection into the target tissue, by inhalation, or by perfusion. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods for Treating Breast Cancer

In further aspects of the present invention, the pharmaceutical compositions described herein may be used for treatment of breast cancer. Within such methods, pharmaceutical compositions are typically administered to a patient. A patient may or may not be afflicted with cancer. Accordingly, the above pharmaceutical compositions may be used to prevent the development of breast cancer or to treat a patient afflicted with breast cancer. Breast cancer may be diagnosed using criteria generally accepted in the art, including the detection method described herein. Pharmaceutical compositions may be administered either prior to or following surgical removal of primary tumors and/or treatment such as administration of radiotherapy or conventional chemotherapeutic drugs.

Routes and frequency of administration of the pharmaceutical compositions described herein, as well as dosage, will vary from individual to individual, and may be readily established using standard techniques. In general, an appropriate dosage and treatment regimen provides the pharmaceutical composition(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### EXAMPLES

The following Examples are offered by way of illustration and not by way of limitation.

### Example 1

### Identification of Genes Differentially Expressed Between the Metastatic Breast Cancer Cell Line MDA-MB-231 and the Non-Tumorigenic Derivative MDA/H6 Using High Density Gene Filters

Total RNA was extracted from MDA-MB-231 and MDA/H6 cells with Trizol Reagent (15596-026, Life Technologies, Rockville, Md.) following the manufacturer's instructions. Briefly, cells were lysed by adding 17.5 ml Trizol solution per 175 cm² flask. After transferring the lysate into a tube, 0.2 ml chloroform was added per 1 ml Trizol reagent used. The samples were centrifuged at 12,000 g for 15 min at 4° C. The aqueous phase was transferred to a fresh tube, and 0.9 ml isopropyl alcohol was added each ml of aqueous phase collected. The samples were incubated at room temperature for 10 min and spun at 12,000 g for 10 min at 4° C. The supernatant was removed and the RNA pellet was washed once with 75% ethanol alcohol. The pellet was air-dried and then dissolved in RNase-free water (D-5758, Sigma, St. Louis, Mo.). RNA was purified using Rneasy Midi Kit 50 (75144, Qiagen, Valencia, Calif.) following manufacturer's instructions. Briefly, 500 μg total RNA was purified by use of 1 mg purification column. The RNA was equalized to 1 ml Rnase-free water and then 3.8 ml Buffer RLT was added. Next, 2.8 ml 100% ethanol alcohol was added and the sample was placed on the Rneasy midi spin column. The column was centrifuged for 5 min at 5,000 g, and the flow-through was discarded. Two and a 0.5 ml Buffer RPE was added to the column that was centrifuged at 5,000 g for 5 min., and repeated once. The column was transferred to a new collection tube and 250 μl RNase-free water was added to the column and spun at 5,000 g for 5 min. This elution step was repeated once. Both of the elution were transferred into a Microcon 100 column and spun at 500 g for 12 min. The column was inversely placed into a tube and spun at 3,000 g for 3 min. to collect the concentrated RNA.

High density gene filters (gf200, gf201, gf202, gf203 and gf211) consisting of 25,985 arrayed elements (19,592 unique human genes and 6,393 controls) were purchased from Research Genetics (Huntsville, Ala.). A new gene filter was first washed in boiling 0.5% SDS for 5 min. and then placed in a 35×150 mm roller tube (052-002, Biometra, Tampa) with the DNA side facing the center of the tube. Next, 5 ml hybridization solution (HYB125.GF, Research Genetics), 5 μl Poly(dA) (POLYA.GF, Research Genetics) and 5 μl Cot-1 DNA (15279-011, Life Technologies) were added to the tube, that was placed in a 42° C. hybridization oven for 2 to 4 hours.

DNA for hybridization on gene filter was labeled as follows. Total RNA (0.8 μg) was suspended to 8 μl RNase-free water. Two μl of 1 μg/μl 10-20 mer of Oligo-(dT) (POLYT.GF, Research Genetics) was added to the RNA solution in a tube that was then incubated in a 70° C. for 10 min. Then, the tube was briefly chilled on ice. Next, 6 μl 5× First Strand Buffer (18064-014, Life Technologies), 1 μl of 0.1M DTT (18064-014, Life Technologies), 1.5 μl of 100 mM dNTP (27-2035-02, Amersham Pharmacia), 1.5 μl Superscript II reverse transcriptase (18064-014, Life Technologies) and 10 μl ³³P dCTP (BF1003, Amersham Pharmacia) were added and mixed thoroughly. A count per minute for radioactivity was recorded by use of Scanner QC4000 (Bioscan Inc. Washington, D.C.). The mixture was placed in a 37° C. water bath for 90 min.

The labeled DNA was brought up to 100 μl Rnase-free water and then purified by use of a Bio-Spin 6 chromatography column (732-6002, Bio-Rad, Hercules, Calif.) following the manufacturer's instruction. DNA with more than 5% of α-³³P incorporation was denatured for 5 min in a boiling water bath and added directly to the pre-hybridization. The hybridization was allowed to continue for 15 h at 42° C. The washes were done to the final stringency of 0.5×SSC, 1% SDS at 50° C. for 15 min. The filters were placed on ddH₂O-moistened piece of Whatmann paper (28458-005, VWR, Bridgeport, N.J.), exposed onto a phosphor screen (Molecular Dynamics) for 5 h, and scanned for signals with the Storm 840 Scanner (Molecular Dynamics). The tiff images were transferred to software IPLab/ArraySuite v2.0 (NHGRI/NIH) for identification of differentially expressed genes as described previously (Su et al., Mol. Carcinog., 28:119-127, 2000).

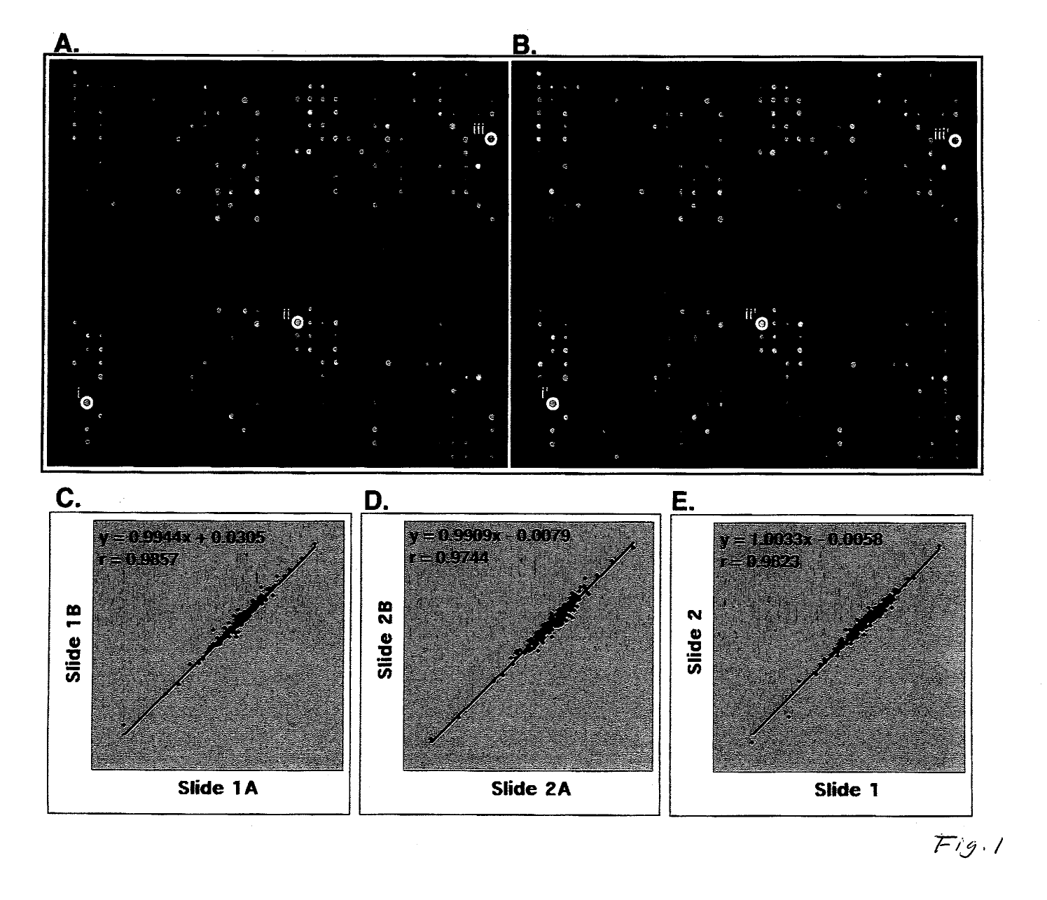

Based on selection criterions of at least 800 expression intensities and 2-fold differences between the two cell lines, 651 of 19,592 genes (3.32%) (FIG. 1, panels C and D) were selected for making microarrays on glass slides to further investigate their expression in multiple breast cancer samples.

### Example 2

### Customized cDNA Microarrays on Glass Slides

In order to reproducibly measure gene expression, the resultant 651 differentially expressed genes and 117 controls were printed as double sets on the individual glass slides. The same batch microarrays were used to measure gene expression in MDA-MB-231 and MDA/H6 cell lines. Briefly, human sequence verified unigene cDNA clones were purchased from Research Genetics. Plasmid DNAs were isolated from bacterial clones. cDNA inserts were amplified by PCR using the vector sequence-specific primers flanking the inserts. 0.21 μg/ml of the purified products including 651 cDNAs, 80 housekeeping genes for ratio control (Chen et al. Biomed. Optics, 2:364-374, 1997), 4 non-specific controls of E. coli DNA, and 33 negative controls of non-DNA sample were printed as double sets on the individual glass slides using GMS417 arrayer (Affymetrix).

The first strand cDNA was labeled by using MicroMax Kit (NEN, Boston, Mass.) following the manufacturer's instruction. All cancer samples were labeled with the fluorescent Cy3-dUTP and the reference sample (MDA/H6) with Cy5-dUTP. Very briefly, 50-ug total RNA was mixed with Cy3-dUTP (or Cy5-dUTP) and other reagents from the kit to synthesize the label first strand cDNA at 42° C. for h. The reaction was stopped by addition of 2.5 ul 0.5M EDTA and 2.5 ul 1N NaOH and then incubated at 65° C. for 30 min. After adding 6.2 ul 1M Tris-HCl (pH 7.5), the samples were purified by use of Microcon 100 (Cat. No. 42412, Millipore Corp., Bedford, Mass.) to remove unincorporated nucleotides and salts. The Cy3- and Cy5- labeled DNA samples of each pair were dissolved into 25 μl Hybridization Buffer from the kit by heating at 50° C. for 10 min. After overlaying a cover slip onto a microarrayed glass slide, the DNA sample was heated at 90° C. for 2 min. After a quick spin, 25-ul sample was placed onto the edge of the coverslip. The sample was drawn underneath the coverslip by capillary action. Each slide was placed in a 50-ml conical tube with moisture Kimwipe. Hybridization was allowed to proceed at 65° C. for 16 h. The slides were washed to a final stringency of 0.06×SSC at room temperature for 15 min.

Image and Statistic Analysis

Hybridized array slides were scanned by use of GenePix 4000A Laser Scanner (Axon Instruments, Inc., Foster City, Calif.). For each slide, two fluorescent intensities (Cy3, Cy5) were scanned separately and then placed into the red and green channel as the tiff images in software IPLab/ArraySuite v2.0 (NHGRI, NIH) for analysis.

Image segmentation, target detection and ratio calibration methods were employed to report the expression ratios of each gene on the slides (Sorlie Proc. Natl. Acad. Sci. U.S.A, 98:10869-10874, 2001). The ratio calibration on gene filters were performed based on signal intensities of all the targets; whereas the ratio calibration on glass slides were conducted based on 80 pre-selected internal control genes of which ratios were normalized close to a value of 1.0. A 99% confidence interval was used to determine significantly up- and down-expressed genes. In addition, an empirically determined intensity filter (greater than 800 on gene filters or greater than 2,000 of average intensities in red or green channels on glass slides, for an intensity range from 0 to 65,535) was applied to further strengthen the stringency for analysis. Scatter plots were drawn in which the calibrated ratios of genes from one set were plotted against those of the other on a log-scale. The linear regression and Pearson coefficient of correlation computed from the scatter plots were used to interpret the strength of the relations of gene expression detected by two sets of genes on the same slides and by genes on two different slides. Multidimensional scaling analysis was performed by use of software developed under MatLab 5.2.1 (The MathWorks, Inc.) platform for the Mac computer. Hierarchical dendrogram clustering analysis was conducted by using the software Cluster/TreeView (Eisen et al. Proc. Natl. Acad. Sci. U.S.A, 95:14863-14868, 1998).

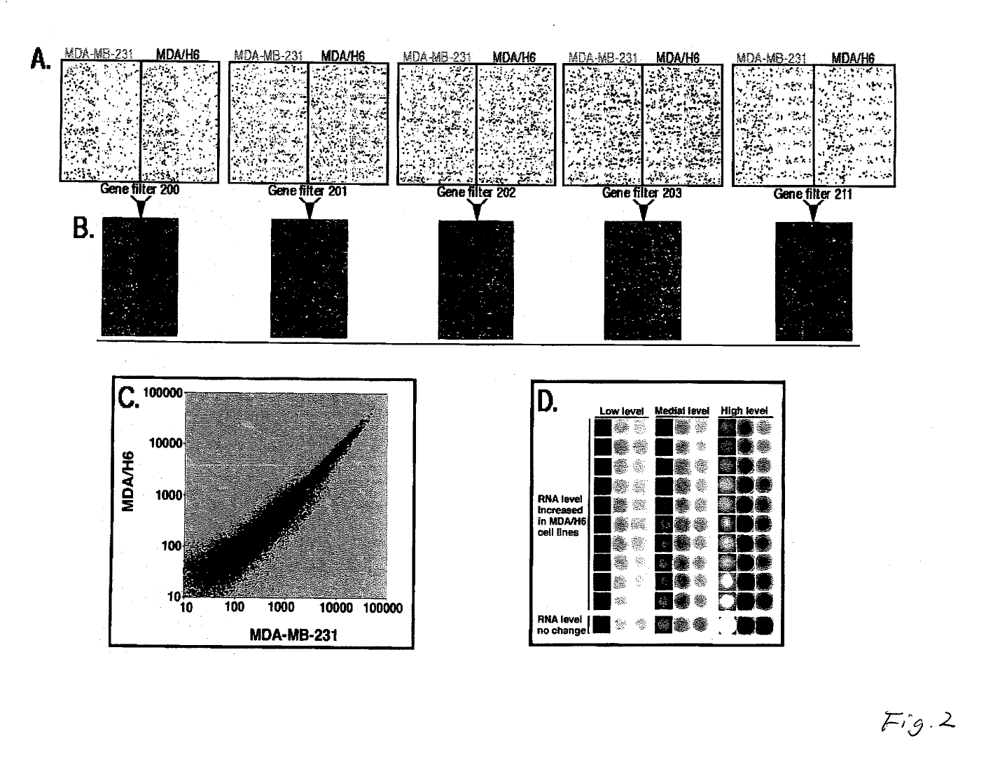

Panels A and B of FIG. 2 show the representative image of 2 sets of genes on the same slide. The calibrated expression ratios of informative genes (>2,000 average intensities in either red or green channel) from these two cell lines were subjected to log-transformation to obtain approximate normal distribution. The log-transformed ratios from one set of genes were drawn against those from the other as a scatter plot, from which a linear regression and Pearson coefficient of correlation were computed. Panels C and D of FIG. 2 show the strong positive linear relations between Set A and Set B on Slide 1 and Slide 2, respectively. In addition, Pearson coefficient of correlation between the Set A and the Set B on Slide 1 and Slide 2 were 0.986 and 0.974, respectively. The expression ratios of genes from Set A and Set B were averaged for the same slides. The average values from Slide 1 were plotted against those from Slide 2. The results indicated, again, a strong positive linear relation with the high value of Pearson coefficient of correlation (r=0.982) (Panel E, FIG. 2), demonstrating the strength of reproducibility of the slides and the experiments.

### Example 3

### Gene Expression Profile of 13 Breast Cancer Samples

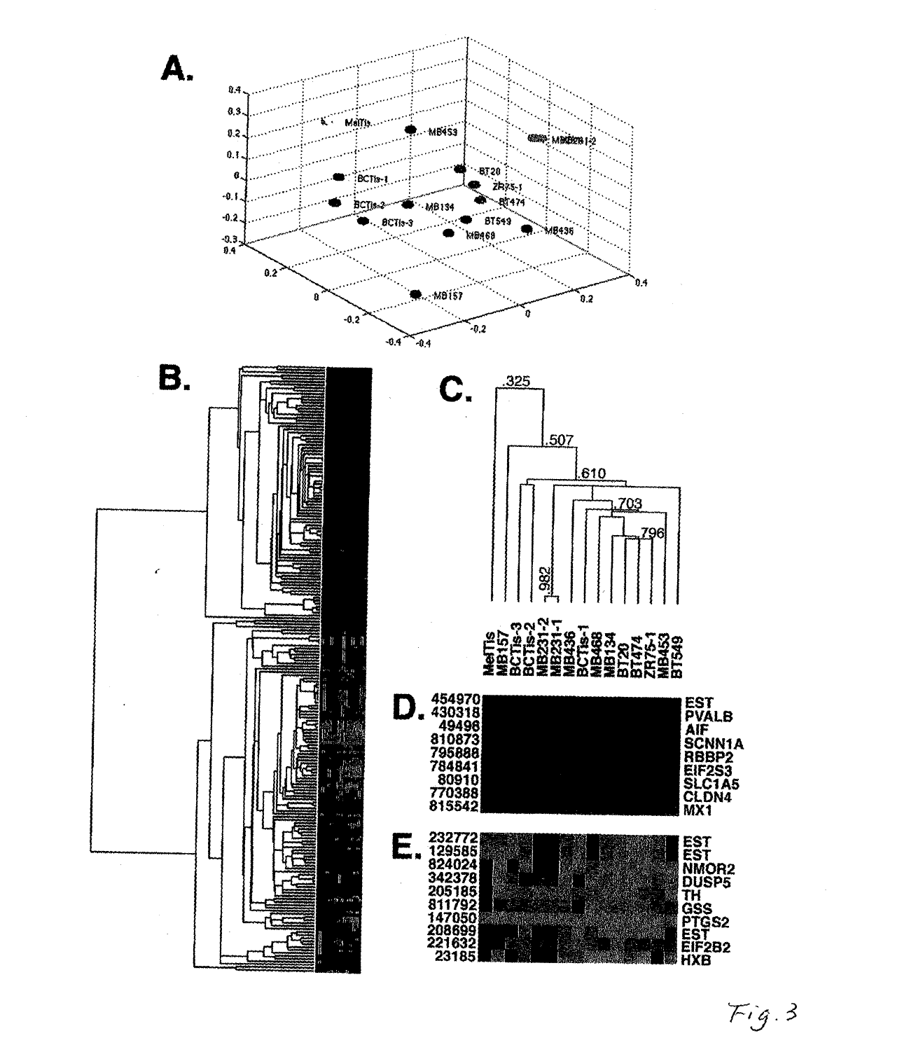

The high quality cDNA microarrays were used to measure expression of 768 arrayed elements (651 differentially expressed genes and 117 controls) in 13 malignant breast cancers using the non-tumorigenic cell line MDA/H6 as a common reference. RNA samples were purified from breast cancer cell lines (n=10) and breast cancer tissues (n=3) (Table 3) and labeled by Cy3-dUTP for microarray hybridization. The reference MDA/H6 samples were labeled with the Cy5-dUTP. An additional MDA-MB-231 sample and a melanoma sample were used as controls for identity and dissimilarity, respectively. Thus, a total of 15 experiments were performed. Out of 731 arrayed human genes, 202 (27.63%) passed the screening filter of the average intensities of genes in red or green channel greater than 2,000 in the range from 0 to 65,535. The expression ratios of the 202 genes were used to compute Pearson coefficient of correlation (or similarities and dissimilarities) among the samples and among the genes. The relative relations of these cancer samples were visualized by multidimensional scaling analysis (MDS, Panel A, FIG. 3) and hierarchical clustering analysis (Panel C, FIG. 3). Panel B of FIG. 3 shows the gene dendrogram from the hierarchical clustering analysis. These results revealed that, first, the expression profiles of two MDA-MB-231 samples were essentially identical (r=0.9823) and that, secondly, the expression pattern of the melanoma sample was the most dissimilar to that of the MDA-MB-231 (r=0.325), as expected. Thirdly, the expression patterns of all other breast cancer samples were distributed between the identical and dissimilar controls (MDA-MB-231 and melanoma). Finally, Pearson coefficients of correlation between breast cancer cell lines BT20, BT474 and ZR75-1 were 0.796, indicating their similarities.

### Example 4

### Frequently Differentially-Expressed Genes

Microarray gene expression analysis revealed 19 genes with high frequent alterations in their expression in human breast cancers. Out of 202 genes with informative expression levels, 9 were highly over-expressed (Panel D, FIG. 3) and 10 were significantly down-regulated (Panel E, FIG. 3) in at least 10 of 13 breast cancer samples. Twenty-one had no significant changes in expression in all 13 breast cancer samples and the remaining 162 genes displayed more than 2 fold changes in at least 1 of 13 samples studied. The nine up-regulated genes are listed in Table 4. The ten down-regulated genes are listed in Table 5.

### Example 5

### The Decrease of the THBD Protein in Breast Cancer Cell Lines and Tissue Specimens

The microarray analysis showed a range from 3 fold to more than 10 fold down-regulation of the THBD RNA in all 13 human breast cancers studied (Panel A, FIG. 4). In order to determine the levels of the THBD protein, Western blot analysis was performed on the breast cancer cell lines MDA/H6, MDA-MB-231, MDA-MB-436, MDA-MB-453, and BT549 (Panel B, FIG. 4). Briefly, cells at 80% confluenc were rinsed twice with ice-cold PBS, scraped into a microcentrifuge tube and pelleted by centrifugation at 6,000 rpm at 4° C. for 3 min. The cell pellets were resuspended in 500 μl Lysis Buffer (1% NP40, 1% sodium deoxycholate, 0.1% SDS, 150 mM NaCl, 0.01M Na₂HPO₄, pH7.4, 1 μg/ml proteinase inhibitors). The lysate were spun at 14,000 rpm at 4° C. for 5 min, after which the supernatants were transferred to a fresh ice-chilled microcentrifuge tube. Protein was then assayed using the Pierce BCA Protein Assay kit (Microwell Plate Protocol) (Pierce, Cat# 23225, Rockford, Ill.). For each sample, the protein concentration was adjusted to 10 μg/μl. Five μl of each sample was mixed with equal volume of 2× loading dye (SeeBlue Pre-Stained Standard, Cat# LC5625, Invitrogen), heated for 5 min at 95° C. and then loaded onto the 8% SDS-polyacrylamide gel (Cat# EC6045, Invitrogen) in the Minigel apparatus (XCELLII, Cat# EI9051, Invitrogen). The gel was run at 150V for 1-1.5 h. The proteins were transferred from the gels to nitrocellulose membrane by use of blotting pads (XCELLII Blotting, Cat# EI9052, Invitrogen) for 1 h under 30V. The membranes were submerged in blocking solution (2.5 g non fat dry milk, 47.5 ml 1× TBS and 20 μl Tween 20) for 1 h at room temperature. The membrane was then rinsed with the blocking solution, and then incubated in the solution of polyclonal goat antibody of thrombomodulin (1:200 dilution with the blocking solution) (Cat# SC-7096, Santa Cruz Biotechnology, Santa Cruz, Calif.) for 1 h at room temperature. The primary antibody was rinsed off with washing solution (49.95 ml 1× TBS and 25 μl Tween 20) three times for 5 min each. The membrane was then incubated in the solution of anti-goat-IgG-HRP (1:1,000 dilutions) (Cat# sc-2056, Santa Cruz Biotechnology) for 1 h at room temperature. The secondary antibody was washed off with the washing solution for 3 times, 10 min each and once with 1× TBS for 15 min. The membrane was incubated in an enhanced chemiluminescent substrate (Pierce Supersignal Chemiluminescent Substrate, Cat# 34080, Pierce, Rockford, Ill.) for min, wrapped in Saran Wrap, and exposed to Kodak X-Omat AR film at room temperature for 2 sec to 1 min. The goat polyclonal IgG of actin I-19 (Cat# sc1616, Santa Cruz Biotechnology) was used as a loading control.

The results demonstrated the high level of the THBD protein in non-tumorigenic breast cancer cell line MDA/H6. In contract, it was decreased approximately 5 folds in MDA-MB-231 and 3 folds in MDA-MB-453, and was not detectable in MDA-MB-436 and BT549. Thus, the results correlated the THBD RNA levels to the protein expression, that is, both of the RNA and the protein were decreased in the breast cancer samples.

In situ immunohistochemical staining for THBD protein was conducted on 20 cases of breast normal and cancer tissue specimens in order to determine THBD protein levels in vivo. Briefly, the tissue sections on slides were incubated at a 60° C. for 1 h, and then immersed in Xylenes (X5-500, Fisher Healthcare, Hanover Park, Ill.) at room temperature for 5 min, twice. The slides were re-hydrated by immersing consecutively in 100%, 75% and 50% ethanol alcohol at room temperature, 2 min in each solution and twice per solution. The slides were rinsed with ddH₂O for 5 min and then immersed into 10 mM Sodium Acetate buffer (pH: 6.0) in a plastic box that was incubated in boiling water for 10 min. All the following procedures were carried out at room temperature. The slides were rinsed with 1× Phosphate Buffered Saline (PBS) (Fisher Healthcare, Hanover Park, Ill.) for 5 min, and then incubated in 3% peroxide (Fisher Healthcare, Hanover Park, Ill.) for 10 min. After washed with 1× PBS buffer for 3 min, twice, the slides were mounted on Shandon chamber coverslip (Shandon Inc, Pittsburgh, Pa.). From now on, the slides were washed with Cadenza Buffer (407340, Shandon, Inc.) for 4 min, referring as washing in the following procedures. Two hundred μl of Protein Block (HK112-9K, BioGenex, Inc.) was placed onto each slide, incubating for 20 min. TM(C-17), an affinity purified goat polyclonal antibody against a peptide at the carboxyl terminus of human thrombomodulin (Santa Cruz, Inc.), was diluted with 1% BSA and 0.01% NaAzide solution to 200- 400 folds. After washing the slides, 200 μl of the diluted antibody was dropped onto each slide, incubating for 1 h. Then, the sections were processed in the following order: incubation in 200 μl anti-immunoglobulin (HK340-9K, BioGenex, Inc.) for 20 min, washing, incubation in 200 μl peroxidase-conjugated streptavidin (HK330-9K, BioGenex, Inc.) for 20 min, washing, incubation in 200 μl DAB (3,3′-diaminobenzidine) Chromogen (HK153-5K, BioGenex, Inc.) for 10 min, and washing. Each slide was counterstained with 300 μl of hematoxylin (HK100-9K, BioGenex, Inc.) for 4 min and then rinsed with ddH₂O for 3 min. The sections were dehydrated by immersing consecutively in 50%, 75%, and 100% ethanol alcohol for 1 min, twice in each solution. After rinsing in Xylenes for min, twice, the slides were mounted for visualization under microscope. Negative controls were processed in the same procedures as above in the absence of the antibody TM(C-17).

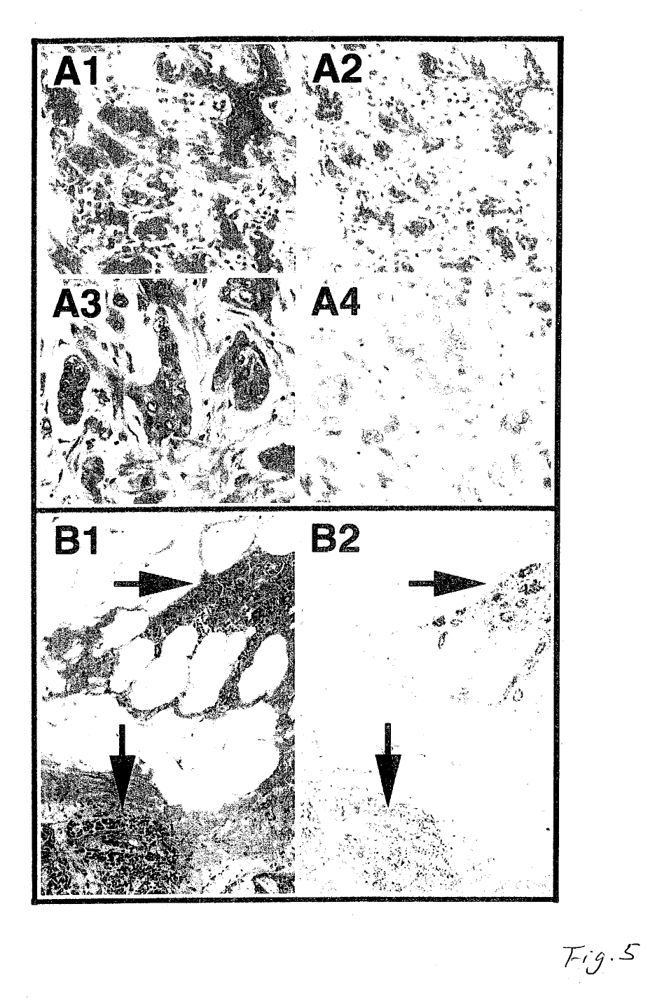

The in situ immunohistochemical staining demonstrated strong positive THBD stain in normal mammary epithelial cells and negative in breast cancer cells (FIG. 5). The control staining for both normal and breast cancer sections without the antibody were negative. Table 6 summarizes the results that 18 out of the 20 cases, including all 5 metastatic breast cancer samples and 13 infiltrating ductal carcinoma samples, lost the THBD protein in the cancer cells, and one case of moderately well differentiated infiltrating adenocarcinoma and one case of infiltrating ductal carcinoma with intramammary lymphatic invasion had the cancer cells with the positive THBD stain. Thus, the results indicated that the THBD protein were absent in advanced breast cancers.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for detecting breast cancer in a subject, said method comprising the steps of:
(a) contacting a biological sample from the subject with an agent that binds to a polypeptide comprising an amino acid sequence recited in any one of SEQ ID NOS:20-38;
(b) determining a level of binding of said agent to said polypeptide;
(c) comparing the level of binding of said agent to said polypeptide to a control level of binding; and
(d) producing a diagnosis based on a result from step (c).

2. The method of claim 1, wherein said agent is an antibody directed against said polypeptide.

3. The method of claim 2, wherein the antibody is selected from the group consisting of Fab fragment, Fab₂ fragment, single chain antibody, chimeric antibody, monoclonal antibody and polyclonal antibody.

4. The method of claim 1, wherein the level of binding of said agent to said polypeptide in said biological sample is determined using a technology selected from the group consisting of ELISA, microarray technology, and biochip technology.

5. The method of claim 1, wherein said agent binds to a polypeptide comprising an amino acid sequence recited in SEQ ID NO:29.

6. A method for detecting breast cancer in a subject, said method comprising the steps of:
(a) determining a level of a transcribed polynucleotide in a biological sample from said subject, wherein said transcribed polynucleotide comprises a nucleic acid sequence recited in any one of SEQ ID NOS:1-19, or a complement of any of the foregoing nucleic acid sequences;
(b) comparing the level of said transcribed polynucleotide in said biological sample to a control level of said transcribed polynucleotide; and
(c) producing a diagnosis based on a result from step (b).

7. The method of claim 6, wherein said transcribed polynucleotide is an mRNA, and wherein the level of mRNA in said biological sample is determined using a method selected from the group consisting of Northern hybridization, RT-PCR, microarray technology, and biochip technology.

8. The method of claim 6, wherein the transcribed polynucleotide comprises a nucleic acid sequence recited in SEQ ID NO:10, or a complement thereof.

9. A method for detecting breast cancer in a subject, said method comprising the steps of:
(a) determining an expression pattern of two or more breast cancer-specific markers in a biological sample from said subject;
(b) comparing the expression pattern of the two or more breast cancer-specific markers in said biological sample to a control expression pattern; and
(c) producing a diagnosis based on a result from step (b),
wherein said breast cancer-specific marker is a polynucleotide comprising a nucleic acid sequence recited in any one of SEQ ID NOS:1-19 or a polypeptide comprising an amino acid sequence recited in any one of SEQ ID NOS:20-38.

10. The method of claim 9, wherein the expression pattern of transcribed polynucleotides in the biological sample is determined using a method selected from the group consisting of Northern hybridization and RT-PCR.

11. The method of claim 9, wherein the expression pattern of polypeptides in the biological sample is determined using antibodies directed against the polypeptides.

12. The method of claim 9, wherein the expression pattern of two or more breast cancer-specific markers is determined using microarray or biochip technology.

13. A pharmaceutical composition for preventing or treating breast cancer, comprising pharmaceutically acceptable carrier and an agent capable of modulating an activity of a breast cancer-specific marker or an expression level of a breast cancer-specific gene,
wherein said breast cancer-specific marker is a polynucleotide comprising a nucleic acid sequence recited in any one of SEQ ID NOS:1-19 or a polypeptide comprising an amino acid sequence recited in any one of SEQ ID NOS:20-38, and
wherein said breast cancer-specific gene is any one of the genes listed in Tables 4 and 5.

14. A method for preventing or treating breast cancer in a subject, said method comprising the step of:
introducing into the subject an effective amount of the pharmaceutical composition of claim 13.

15. A method of identifying an agent capable of binding to a breast cancer-specific marker, said method comprising:
contacting a breast cancer-specific marker with a candidate agent; and
determining a binding affinity of said candidate agent to said breast cancer-specific marker,
wherein said breast cancer-specific marker is a polynucleotide comprising a nucleic acid sequence recited in any one of SEQ ID NOS:1-19 or a polypeptide comprising an amino acid sequence recited in any one of SEQ ID NOS:20-38.

16. The method of claim 15, wherein the breast cancer-specific marker or the candidate agent contains a label.

17. A method of identifying an agent capable of modulating an activity of a breast cancer-specific marker, comprising:
contacting a breast cancer-specific marker with a candidate agent;
determining an activity of said breast cancer-specific marker in the presence of said candidate agent;
determining the activity of said breast cancer-specific marker in the absence of said candidate agent; and
determining whether said candidate agent affects the activity of said breast cancer-specific marker,
wherein said breast cancer-specific marker is a polynucleotide comprising a nucleic acid sequence recited in any one of SEQ ID NOS:1-19 or a polypeptide comprising an amino acid sequence recited in any one of SEQ ID NOS:20-38.

18. A biochip comprising any one of:
(a) a polynucleotide comprising a nucleic acid sequence recited in any one of SEQ ID NOS:1-19;
(b) a variant of the polynucleotides of (a);
(c) a polypeptide comprising an amino acid sequence recited in any one of SEQ ID NOS:20-38; and
(d) a variant of the polypeptide of (c),
wherein the biochip is utilized for diagnosing breast cancer or screening agents that inhibit breast cancer.

19. A kit for diagnosing breast cancer, said kit comprising a polynucleotide probe or an antibody, wherein said polynucleotides probe specifically binds to a transcribed polynucleotide comprising a nucleic acid sequence recited in any one of SEQ ID NOS:1-19, and wherein said antibody is capable of immunospecific binding to a polypeptide comprising an amino acid sequence recited in any one of SEQ ID NOS:20-38.

20. The kit of claim 19, wherein the polynucleotides probe specifically binds to a transcribed polynucleotide comprising a nucleic acid sequence recited in SEQ ID NO:10, and wherein the antibody is capable of immunospecific binding to a polypeptide comprising an amino acid sequence recited in SEQ ID NO:29.


SEQUENCE LISTING (38 sequences)

SEQ ID NO: 1 - DNA, 2787 bp, Homo sapiens
agagcggaggccgcactccagcactgcgcagggaccgccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcag

SEQ ID NO: 2 - DNA, 424 bp, Homo sapiens
accagcccagcctttcagtgcaggctccagccctccacccccacccgagttgcaggatgtcgatgacagacttgctgaacgctgaggacatcaagaaggcggtgggagcctttagcgctaccgactccttcgaccacaaaaagttcttccaaatggtcggcctgaagaaaaagagtgcggatgatgtgaagaaggtgtttcacatgctggacaaggacaaaagtggcttcatcgaggaggatgagctgggattcatcctaaaaggcttctccccagatgccagagacctgtctgctaaagaaaccaagatgctgatggctgctggagacaaagatggggacggcaaaattggggttgacgaattctccactctggtggctgaaagctaagaagcactgactgcccctggtcttccacctctctg

SEQ ID NO: 3 - DNA, 6455 bp, Homo sapiens
gaggaagcgattctggggtttctgtgttgaacggttcttgtccgcgaagatgcgcttcggcctctgtcaggggacttgaacgggcttagtgggcttcagccagcttttctccaccggttccccacggggaccccccccccccggccgttgcaatggcgggcgtggggccggggggctacgcggcggagttcgtgccaccgccagagtgccccgtctttgagccgagttgggaggagttcacagatccgctcagctttatcggccgcatccggcctttggcggagaaaaccggcatctgcaaaattcggccgcccaaggactggcagcctccatttgcctgtgaagtaaaaagctttcgtttcactccaagagtccagcgcctgaatgaacttgaggcaatgaccagagtgagattggatttcttggatcaactagcaaaattttgggaacttcaaggatctactctgaagatccctgtggtagagagaaaaatcctggatctgtatgctttgagcaagattgttgccagcaaaggaggttttgaaatggtcaccaaagagaagaaatggtctaaagtgggtagtcgcttgggatatctgccaggaaaaggaactgggtctcttttgaagtcacattatgaaagaattctctacccatatgagcttttccagtctggtgtgagccttatgggtgtgcagatgcctaatttagatcttaaagaaaaagtggagcctgaggttctcagcactgatacccaaacttccccagagccaggcacaaggatgaacattctgccgaagagaacaagacgtgtgaagactcagtcagaatctggagatgtgagtagaaacacggaactgaagaaacttcagatttttggggctgggcccaaggttgtgggcttggcaatgggaacaaaagataaagaagatgaggtcacccgaagacgaaaagttaccaacaggtcagacgcatttaacatgcaaatgagacaacggaaaggcactctctctgttaactttgttgatctctatgtttgtatgttttgtggtcggggaaacaatgaagataaattgcttttgtgtgatggatgtgatgacagctatcatacattttgtctaattcctccactacctgatgtgcccaaaggagactggaggtgtcctaaatgtgtcgccgaggaatgtagcaaacctcgagaagcctttggatttgaacaagctgtacgagagtatacacttcagagctttggagagatggcagataattttaagtctgattattttaatatgccagtccatatggttcccacagaactagtagaaaaggaattttggcggctggtaagcagcattgaagaagatgttattgtggaatatggagcagatatctcctcaaaagactttggaagtggatttccggtgaaggatgggcggagaaagattctgccagaagaagaggaatatgcactttctggttggaatttgaataacatgcctgtcctggaacagtctgttcttgcacatattaatgtggacatctctggtatgaaagtgccgtggctctatgtgggaatgtgcttctcttctttttgctggcacattgaggatcactggagttattccatcaactacttgcactggggggagccaaagacatggtatggtgtgccatctcatgctgcagagcaactggaggaggtgatgagagagctggcccccgagttatttgaatcccagcctgatcttctgcatcagttagttaccatcatgaaccccaacgtgctaatggagcatggtgtgcctgtgtacaggaccaatcagtgtgctggcgagtttgttgtgacatttcctcgtgcctatcactctggatttaaccagggctacaactttgctgaagctgtgaacttctgtactgctgactggttgcccattggacgtcaatgtgtaaatcattaccgacgcctaaggcgccactgtgtcttttcacacgaggaactaattttcaagatggcagcagatccagaatgcttagatgtggggctggctgccatggtctgcaaagaattgactctcatgactgaagaagaaacacgattaagagagtctgttgtacagatgggtgtcctgatgtcagaagaagaagtgtttgaacttgttcctgatgatgagcggcagtgttcagcatgcagaaccacatgttttctctctgctctcacatgttcctgtaatcctgagcggcttgtatgtctctaccatccaactgatctgtgcccctgccccatgcagaagaaatgtcttagatatcgctacccattagaagacctcccttctctgctatatggtgtaaaagtcagggcacagtcctatgacacttgggtcagtcgtgttacagaagcattgtctgctaacttcaaccacaaaaaagatttgattgaattgcgagtaatgctggaagatgctgaggataggaaatacccagagaatgatctctttcgaaaactcagggatgctgtaaaagaagctgagacctgtgcttctgtggctcagctgcttctgagcaaaaagcagaaacacagacagagcccagatagtgggaggactcggaccaaactgacagtggaagaattgaaggcctttgtccaacaactttttagtcttccgtgtgtcatcagccaagctcggcaagtaaagaatctgctagatgatgtggaagagtttcatgaacgtgctcaggaggccatgatggatgaaaccccagattcttccaaactccagatgttgatagatatgggctctagtctctatgtggaactccctgaattaccacgactgaagcaagagctacaacaggctcggtggttggacgaagtaagactgaccttatcagatccgcaacaagtcactttggatgtcatgaagaagctgatagactctggggtagggttggcaccccaccatgctgtggagaaagcaatggctgaactacaggagctccttacagtctctgaacgatgggaagaaaaggctaaggtctgcctacaggcaagaccgaggcacagtgtggcaagtttagaaagcattgtgaatgaagccaagaacattccagcctttctacccaatgtgttgtccttgaaagaagccttacaaaaggctcgagaatggaccgctaaagtggaagctattcagagtggcagcaattacgcttatttggagcagcttgagagcttgtctgcgaaaggacgccctattcctgtgcgtcttgaagcactgccgcaagtggaatcacaggtagcagcagcacgggcatggagagaacggactgggcggacgtttcttaagaagaattctagccatacattgttacaggtgctgagcccccggaccgacattggtgtatatgggagtggcaaaaataggaggaaaaaagtaaaagaactaatagaaaaagaaaaagaaaaggatctggacctggagcctctgagtgatctggaggaaggattggaggaaaccagagatacagccatggtggtggcagttttcaaagaacgggagcaaaaagagattgaagccatgcattctctcagagcagccaacctagccaagatgacaatggtggaccgcatagaagaagtaaaattttgcatttgccgcaagacagccagtgggtttatgctacagtgtgagctctgcaaagactggttccataacagctgtgttcctcttcctaaatcaagttcccaaaaaaaaggatccagctggcaagctaaagaagtaaaattcctttgccctctttgtatgcggtctcgaaggcccaggctagagactattctgtcactcctggtatcccttcagaagttgcccgtacggttgcctgaaggagaggccctgcagtgtttgacagaacgtgctatgagttggcaagatagagcgcggcaggctctagccacagatgaactatcctctgccctggccaaactatctgtgttgagccagcgtatggtggaacaggcggctcgagaaaaaactgaaaagatcatcagtgcagaactccaaaaagcagctgccaatccagacttacagggacacttacctagtttccagcagtctgcttttaaccgggtggtgagcagtgtgtcatcttctcctcgacaaacaatggactatgatgatgaagaaacagactctgatgaagacattcgagagacatatggctacgacatgaaggacacagccagtgtgaagtcctctagtagtcttgaacccaatcttttttgtgatgaagagattcccatcaaatccgaggaggtggtgacccacatgtggacagcaccttcattttgtgcagagcatgcttattcttctgcttctaagagttgttctcaagtattttttgggaaaggttctagcaccccaaggaaacaacctcggaagagccctttggtgccccgaagtttggaacctccagtgctggagttgtcacctggagctaaggcacaactggaagaacttatgatggttggagatctcctggaagtatctctggacgagactcaacacatatggcggattttgcaggccacacacccaccctctgaagacagattcttgcatatcatggaggatgacagcatggaagagaaaccactaaaagtgaaaggaaaggactcttcagagaagaaacggaaacggaagctagaaaaggtagagcaactttttggagaaggaaaacagaagtccaaggagttaaagaaaatggacaaacctagaaagaagaaattaaaattaggtgcagacaaatcaaagaagctgaataaactggccaagaaactagcaaaagaagaagagagaaagaaaaagaaggagaaggctgctgcagccaaagttgaacttgtgaaagagagcactgaaaagaaaagagagaaaaaggtgctggacatcccctcaaagtatgactggtcaggagcagaggagtctgatgatgagaatgctgtgtgcgcagaaccagactgccaaaggccctgcaaggacaagggagttgtatttgtaacgaagaagagagagataaaaaatattagttttaaaagtgtcctatgtgactgcttttctaaaaaggtagactgggtacaatgtgatggtggctgtgatgagtggtttcatcgggtttgtgtgggtgtatctccagaaatggctgaaaatgaagattacatctgtataaactgtgcaaagaagcaggggccagttagcccaggtccagcaccacctccttccttcataatgagctacaaactaccaatggaggatcttaaagagaccagttagcagatgcttggttagtttgggacatggggggacatggaccacattgagaccttagtcatcaagtagagtggtttatatcacttggaatgttgcttctaaagatgaatggccttcagagaaagtccccttagtgctggcttcctctttgcatggactctgtgggttacattgctctatcaacatatctatgcagagggtgtcttctttggtacaacagccaatatctcatgtctcctttgagtgtggtttactgcattaaggccagatgcttaattgagctctagggtggctggttagtattaatacattggtgtgctaacagggcatataggatgtggcttttgtccagctgatagtagttagaggcttacaacttaggagcagcaccaactgaaggtgctaattgcttggatctccttcattaggatagttggagagggattggagtaccactttcttccactgttaccaggtacttaatgccctaaagatacaactaggagtaacagggccaaagttatttctgttagacgtcaaggaatggtatcacagtctattgacctcagcgatttgtgcttgtttgtgctagaagaacatcccaaataggagaacctctcacaagctggggcaggtcaccttatctttgtaagatgaggatatcatctagatcagaaatctgactagattggattctgaggagaagaacctactacaaggcaaggagccgttttttggctttgaaaagtcttgctgtcttgggtctacattttagggaagagcaggtacatggatccaggcttctgccaaaaaaaaaaagagaagaagatgacgagtatgaccagtcgtactatcttactgagccacagtgatgcatgcttttcggggaaaacttcattcacaagtattccagacaccaggcttcaggcatggccatgagcaagaccagcaaataacagctttttcccttgcagccctgaccccaatgtctgctgtttccaacactggtgatttctaactacggcccacagcagatgctgttgaataacaccatggcttcatcagaggatgtggggttgtagtacctctgggtgatgaagttgttttagcaaatccatttttaaaaaaaaaaaaaaa

SEQ ID NO: 4 - DNA, 1369 bp, Homo sapiens
ggccgacagtgcctgatttgagatggggtcccaggtctcggtggaatcgggagctctgcacgtggtgattgtgggtgggggctttggcgggatcgcagcagccagccagctgcaggccctgaacgtccccttcatgctggtggacatgaaggactccttccaccacaatgtggctgctctccgagcctccgtggagacagggttcgccaaaaagacattcatttcttactcggtgactttcaaggacaacttccggcaggggctagtagtggggatagacctgaagaaccagatggtgctgctgcagggtggcgaggccctgcccttctctcatcttatcctggccacgggcagcactgggcccttcccgggcaagtttaatgaggtttccagccagcaggccgctatccaggcctatgaggacatggtgaggcaggtccagcgctcacggttcatcgtggtggtgggaggaggctcggctggagtggagatggcagcagagattaaaacagaatatcctgagaaagaggtcactctcattcactcccaagtggccctggctgacaaggagctcctgccctccgtccggcaggaagtgaaggagatcctcctccggaagggcgtgcagctgctgctgagtgagcgggtgagcaatctggaggagctgcctctcaatgagtatcgagagtacatcaaagtgcagacggacaaaggcacagaggtggccaccaacctggtgattctctgcaccggcatcaagatcaacagctccgcctaccgcaaagcatttgagagcagactagccagcagtggtgctctgagagtgaacgagcacctccaggtggagggccacagcaacgtctacgccattggtgactgtgccgacgtgaggacgcccaagatggcctatcttgccggcctccacgccaacatcgccgtggccaacatcgtcaactctgtgaagcagcggcctctccaggcctacaagccgggtgcactgacgttcctcctgtccatggggagaaatgacggtgtgggccaaatcagtggcttctatgtgggccggctcatggttcggctgaccaagagccgggacctgttcgtctctacgagctggaaaaccatgaggcagtctccaccttgatggagaggccaggcgggagaactaccgcagcaggtgggcgtacggactgcttggcgcatggcacccgcctggcaagtgctagaactaatgctattcttctggaataagatgccaatgatgtggtggctagaaatgcaacttgtataaaacaaaaatgggagagagagaggtattaaacaaataccccccttagaggataaaaaaaaaaaaaaaaaaaaaaaaaaa

SEQ ID NO: 5 - DNA, 1712 bp, Homo sapiens
ggcacgaggggcagctgtcggctggaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgcggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgtaaggtgccacggctccactctgttcctctctgctttgttcttccctggactgagctcagcgcaggctgtgaccccaggagggccctgccacgggccactggctgctggggactggggactgggcagagactgagccaggcaggaaggcagcagccttcagcctctctggcccactcggacaacttcccaaggccgcctcctgctagcaagaacagagtccaccctcctctggatattggggagggacggaagtgacagggtgtggtggtggagtggggagctggcttctgctggccaggatggcttaaccctgactttgggatctgcctgcatcggtgttggccactgtccccatttacattttccccactctgtctgcctgcatctcctctgttgcgggtaggccttgatatcacctctgggactgtgccttgctcaccgaaacccgcgcccaggagtatggctgaggccttgcccacccacctgcctgggaagtgcagagtggatggacgggtttagaggggaggggcgaaggtgctgtaaacaggtttgggcagtggtgggggagggggccagagaggcggctcaggttgcccagctctgtggcctcaggactctctgcctcacccgcttcagcccagggcccctggagactgatcccctctgagtcctctgccccttccaaggacactaatgagcctgggagggtggcagggaggaggggacagcttcacccttggaagtcctggggtttttcctcttccttctttgtggtttctgttttgtaatttaagaagagctattcatcactgtaattattattattttctacaataaatgggacctgtgcacaggaaaaaaaaaaaaaaaaaaaaaaaa

SEQ ID NO: 6 - DNA, 2163 bp, Homo sapiens
ggcagatgaaatataagattcatcaaccacatttgacagcccatggcaggtttcctgttttccatcgtccctctgcaggtcacagacacacagagcccagccgtggcaggctcagccggggtccggggctgctaacaacggctacattcctcccccagggccaagggaaatcctgagcgcaggccagggttgtttggttttgaggtgtgctgggatgaaaggcaccctggaagtggaaggtaaatgaacaatggaaaaacttcacggcaagattagaaagatacctgagcccaatacccgcctgatgtcgtgggccacacctccgggttaccaggggaagggaggaagcaaactgtcatattgatgtggctctaaacaacaacagtgtgcgaaggcccaggggcactttgggattgaccaagaggaaacacaagttgcacaatgatacaatcttgttggtacaattgtcagagaagggaactcccacagcaaaggccataaaaccatccagggcagtctggggcggctcagttctgcggtgccagggagtggagcagagctcagccccgtcccaaacacagatgggaccatgaactccggacacagcttcagccagaccccctcggcctccttccatggcgccggaggtggctggggccggcccaggagcttccccagggctcccaccgtccatggcggtgcggggggagcccgcatctccctgtccttcaccacgcggagctgcccaccccctggagggtcttggggttctggaagaagcagccccctactaggcggaaatgggaaggccaccatgcagaatctcaacgaccgcctggcctcctacctggagaaggttcgcgccctggaggaggccaacatgaagctggaaagccgcatcctgaaatggcaccagcagagagatcctggcagtaagaaagattattcccagtatgaggaaaacatcacacacctgcaggagcagatagtggatggtaagatgaccaatgctcagattattcttctcattgacaatgccaggatggcagtggatgacttcaacctcaagtatgaaaatgaacactcctttaagaaagacttggaaattgaagtcgagggcctccgaaggaccttagacaacctgaccattgtcacaacagacctagaacaggaggtggaaggaatgaggaaagagctcattctcatgaagaagcaccatgagcaggaaatggagaagcatcatgtgccaagtgacttcaatgtcaatgtgaaggtggatacaggtcccagggaagatctgattaaggtcctggaggatatgagacaagaatatgagcttataataaagaagaagcatcgagacttggacacttggtataaagaacagtctgcagccatgtcccaggaggcagccagtccagccactgtgcagagcagacaaggtgacatccacgaactgaagcgcacattccaggccctggagattgacctgcagacacagtacagcacgaaatctgctttggaaaacatgttatccgagacccagtctcggtactcctgcaagctccaggacatgcaagagatcatctcccactatgaggaggaactgacgcagctacgccatgaactggagcggcagaacaatgaataccaagtgctgctgggcatcaaaacccacctggagaaggaaatcaccacgtaccgacggctcctggagggagagagtgaagggacacgggaagaatcaaagtcgagcatgaaagtgtctgcaactccaaagatcaaggccataacccaggagaccatcaacggaagattagttctttgtcaagtgaatgaaatccaaaagcacgcatgagaccaatgaaagtttccgcctgttgtaaaatctattttcccccaaggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgcagccttcagattctcatcattttgcatctattttgtagccaataaaactccgcactagcaaaaaaaaaaaaaaaa

SEQ ID NO: 7 - DNA, 2856 bp, Homo sapiens
gtaaccgctactcccggacaccagaccaccgccttccgtacacaggggcccgcatcccaccctcccggacctaagagcctgggtcccctgtttccggaggtccgcttcccggcccccagattctggcatcccagccctcagtgtccaagacccaggcagcccgggtccccgcctcccggatccaggcgtccgggatctgcgccaccagaacctagcctcctgcagacctccgccatctgggggcactcaacctcctggagccaagggccccacgtcccacccagagaaactctcgtattcccagctcctagggccaaggaacccgggcgctccgaactcccagctttcggacatctggcacacggggcagagcagagaagctcagcgcccagcctggggaatttaaacactccagcttccaagagccaaggaacttcagtgctgtgaactcacaactctaaggagccctccaaagttccagtctccaggtgctgttactcaactcagtcctaggaacgtcgggtcctgggaaggagcccaagcgctcccagccagcttccaggcgctaagaaaccccggtgcttcccatcatggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattatgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatgtaaaccccgggagggaccttccctgccctgctgggggtgctctttggacactggattatgaggaatggataaatggatgagctagggctctgggggtctgcctgcacactctggggagccaggggccccagcaccctccaggacaggagatctgggatgcctggctgctggagtacatgtgttcacaagggttactcctcaaaacccccagttctcactcatgtccccaactcaaggctagaaaacagcaagatggagaaataatgttctgctgcgtccccaccgtgacctgcctggcctcccctgtctcagggagcaggtcacaggtcaccatggggaattctagcccccactggggggatgttacaacaccatgctggttattttggcggctgtagttgtggggggatgtgtgtgtgcacgtgtgtgtgtgtgtgtgtgtgtgtgtgtgtgtgtgttctgtgacctcctgtccccatggtacgtcccaccctgtccccagatcccctattccctccacaataacagaaacactcccagggactctggggagaggctgaggacaaatacctgctgtcactccagaggacattttttttagcaataaaattgagtgtcaactatttaaaaaaaaaaaaaaaaa

SEQ ID NO: 8 - DNA, 2646 bp, Homo sapiens
tttccttcctcttttggcaacatggcgggcggagaagctggagtgactctagggcagccgcatctttcgcgtcaggatctcaccaccttggatgttaccaagttgacgccactttcacacgaagttatcagcagacaagccacaattaacataggtacaattggtcatgtagctcatgggaaatccacagtcgtcaaagctatttctggagttcatactgtcaggttcaaaaatgaactagaaagaaatattacaatcaagcttggatatgctaatgctaagatttataagcttgatgacccaagttgccctcggccagaatgttatagatcttgtgggagcagtacacctgacgagtttcctacggacattccagggaccaaagggaacttcaaattagtcagacatgtttcctttgttgactgtcctggccacgatattttgatggctactatgctgaacggtgcagcagtgatggatgcagctcttctgttgatagctggtaatgaatcttgccctcagcctcagacatcggaacacctggctgctatagagatcatgaaactgaagcatattttgattctacaaaataaaattgatttggtaaaagaaagtcaggctaaagaacaatacgagcagatccttgcatttgtccaaggtacagtagcagagggagctcccattattccaatttcagctcagctgaaatacaatattgaagttgtttgtgagtacatagtaaagaaaattccagtacccccaagagactttacttcagagccccggcttattgttattagatcttttgatgtcaacaaacctggctgtgaagttgatgaccttaagggaggtgtagctggtggtagtatcctaaaaggagtattaaaggtgggccaggagatagaagtaagacctggtattgtttccaaagatagtgaaggaaaactcatgtgtaaaccaatcttttccaaaattgtatcactttttgcggagcataatgatctgcaatatgctgctccaggcggtcttattggagttggaacaaaaattgaccccactttgtgccgggctgacagaatggtggggcaagtacttggtgcagtcggagctttacctgagatattcacagaattggaaatttcctatttcctgcttagacggcttctaggtgtacgcactgaaggagacaagaaagcagcaaaggttcaaaagctgtctaagaatgaagtgctcatggtgaacataggatccctgtcaacaggagggagagttagtgctgtcaaggccgatttgggtaaaattgttttgaccaatccagtgtgcacagaggtaggagaaaaaattgcccttagccgaagagttgaaaaacactggcgtttaattggttggggtcagataagaagaggagtgacaatcaagccaacagtagatgatgactgaagaataccagttaaataatacattcggatggatttggaagttggaattcctcttaacaaccaaggggtttattttcaaagcaatattggggaattgatttcacagttcgttaccttagtaggtaacggtaaggttattctctttttttttttttttggttatgaaaacttagggactaaaattaatataaaaattggcataatgttggattgaatctacattttggcagaagttaaacattcccacataatgtcaaaattatacatcatgcagttctgtttttttgtttgtttaattttgttttgtttttgagtctggctctgtcacccaggctggagtgcagtggcgtgatctgcaacctctgccccccgggttcaagcgattctcctgcctcagcctcccgagtagctgagattacaggtgcgcgccaccacacttggctaatttttgtattattagtagagacggggtttcagcatgttggctaggccggtctctcctgacctcagggtgatcagcccacctcggcctcacaaagtgctgggattacaggcgtgagccaccttgcccagcccacatcatacagtttgaaatgaaactttgccacaaccagcctttgctgtagcacacacatatatcactgaacctgtttgaaataaagttttttttctttttcatgattcgtctttgagtacctccaggctgaaagactgttgtaccagtaaaaacttaaaggcacaaattctccttgaagaccttctcccttttatgtggccccatattttatgttgctttatctttgaaattttgcatgaaaaggaaatgaatggattcgaatgaaattgtcctttagagcatgattacttgttcccatggacaaatatttttctccccttgctcttcctggcctgaaacacgggaaaccagagtcaaaagttatctccctctccctgtgatgccttgagatttttttctgcgttgtttaatgcctgaaatccaagtcttcctccatgggaaaatactgttataccaaataattctagatgagtaacaaagatctttttaggccttcattttatgttttttcttaactgttatattatgattgtgacatagattatactactactaatttttggatgtttcaaaaggtcaagaagtaaaagatgttagaaagcaaaaaaaaaaaaaaaaaaaaaaaaaaaa

SEQ ID NO: 9 - DNA, 3151 bp, Homo sapiens
ccggccagcgggcgggctccccagccaggccgctgcacctgtcaggggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctgagagggaaggagaggtttctcacaccaaggcagatgctcctctggtgggagggtgctggccctggcaagattgaaggatgtgcagggcttcctctcagagccgcccaaactgccgttgatgtgtggaggggaagcaagatgggtaagggctcaggaagttgctccaagaacagtagctgatgaagctgcccagaagtgccttggctccagccctgtaccccttggtactgcctctgaacactctggtttccccacccaactgcggctaagtctctttttcccttggatcagccaagcgaaacttggagctttgacaaggaactttcctaagaaaccgctgataaccaggacaaaacacaaccaagggtacacgcaggcatgcacgggtttcctgcccagcgacggcttaagccagcccccgactggcctggccacactgctctccagtagcacagatgtctgctcctcctcttgaacttgggtgggaaaccccacccaaaagccccctttgttacttaggcaattccccttccctgactcccgagggctagggctagagcagacccgggtaagtaaaggcagacccagggctcctctagcctcatacccgtgccctcacagagccatgccccggcacctctgccctgtgtctttcatacctctacatgtctgcttgagatatttcctcagcctgaaagtttccccaaccatctgccagagaactcctatgcatcccttagaaccctgctcagacaccattacttttgtgaacgcttctgccacatcttgtcttccccaaaattgatcactccgccttctcctgggctcccgtagcacactataacatctgctggagtgttgctgttgcaccatactttcttgtacatttgtgtctcccttcccaactagactgtaagtgccttgcggtcagggactgaatcttgcccgtttatgtatgctccatgtctagcccatcatcctgcttggagcaagtaggcaggagctcaataaatgtttgttgcatgaaaaaaaaaaaaaaaaaa

SEQ ID NO: 10 - DNA, 4050 bp, Homo sapiens
cttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcacggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcatctagctgtctgctcagacagaacccctacatgaaacagaaacaaaaacactaaaaataaaaatggccatttgctttttcaccagatttgctaatttatcctgaaatttcagattcccagagcaaaataattttaaacaaagggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 11 - DNA, 4465 bp, Homo sapiens
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 12 - DNA, 1856 bp, Homo sapiens
gggagaaccgttcgcggaggaaaggcgaactagtgttgggatggccaccaactgggggagcctcttgcaggataaacagcagctagaggagctggcacggcaggccgtggaccgggccctggctgagggagtattgctgaggacctcacaggagcccacttcctcggaggtggtgagctatgccccattcacgctcttcccctcactggtccccagtgccctgctggagcaagcctatgctgtgcagatggacttcaacctgctagtggatgctgtcagccagaacgctgccttcctggagcaaactctttccagcaccatcaaacaggatgactttaccgctcgtctctttgacatccacaagcaagtcctaaaagagggcattgcccagactgtgttcctgggcctgaatcgctcagactacatgttccagcgcagcgcagatggctccccagccctgaaacagatcgaaatcaacaccatctctgccagctttgggggcctggcctcccggaccccagctgtgcaccgacatgttctcagtgtcctgagtaagaccaaagaagctggcaagatcctctctaataatcccagcaagggactggccctgggaattgccaaagcctgggagctctacggctcacccaatgctctggtgctactgattgctcaagagaaggaaagaaacatatttgaccagcgtgccatagagaatgagctactggccaggaacatccatgtgatccgacgaacatttgaagatatctctgaaaaggggtctctggaccaagaccgaaggctgtttgtggatggccaggaaattgctgtggtttacttccgggatggctacatgcctcgtcagtacagtctacagaattgggaagcacgtctactgctggagaggtcacatgctgccaagtgcccagacattgccacccagctggctgggactaagaaggtgcagcaggagctaagcaggccgggcatgctggagatgttgctccctggccagcctgaggctgtggcccgcctccgcgccacctttgctggcctctactcactggatgtgggtgaagaaggggaccaggccatcgccgaggcccttgctgcccctagccggtttgtgctaaagccccagagagagggtggaggtaacaacctatatggggaggaaatggtacaggccctgaaacagctgaaggacagtgaggagagggcctcctacatcctcatggagaagatcgaacctgagccttttgagaattgcctgctacggcctggcagccctgcccgagtggtccagtgcatttcagagctgggcatctttggggtctatgtcaggcaggaaaagacactcgtgatgaacaagcacgtggggcatctacttcgaaccaaagccatcgagcatgcagatggtggtgtggcagcgggagtggcagtcctggacaacccataccctgtgtgagggcacaaccaggccacgggaccttctatcctctgtatttgtcattcctctcctagccctcctgaggggtatcctcctaaagacctccaaagtttttatggaagggtaaatactggtaccttcccccagctttccatctgaggaccagaaaagttgtgtctcccttagatgagatctagacgcccccaaatccttgagatgtgggtatagctcagggtaagctgctctgaggtaaaggtccatgaaccctgccccactcctgtcagcccctcatcagccttttcagcaggttccagtgcctgacttgggataggactgagtggtaggaggagggggagtggaggggcatagcctttccctaattctgccttaaataaaactgcattgctgt

SEQ ID NO: 13 - DNA, 2473 bp, Homo sapiens
aatcgcgaaacccggcgagcggcgcgctggctatcgagcgagcggggcggaaccgggagttgcgccgccgctcgggcgccgggctccgtcgcggccgcagccccgcgggtcgccctcccgtgcctcgcccgcggacaccctggccgtggacaccctggccgtgggcacccgcggggcgcggcgcgggcgctgcgcggcggcggcggcggcatgaaggtcacgtcgctcgacgggcgccagctgcgcaagatgctccgcaaggaggcggcggcgcgctgcgtggtgctcgactgccggccctatctggccttcgctgcctcgaacgtgcgcggctcgctcaacgtcaacctcaactcggtggtgctgcggcgggcccggggcggcgcggtgtcggcgcgctacgtgctgcccgacgaggcggcgcgcgcgcggctcctgcaggagggcggcggcggcgtcgcggccgtggtggtgctggaccagggcagccgccactggcagaagctgcgagaggagagcgccgcgcgtgtcgtcctcacctcgctactcgcttgcctacccgccggcccgcgggtctacttcctcaaagggggatatgagactttctactcggaatatcctgagtgttgcgtggatgtaaaacccatttcacaagagaagattgagagtgagagagccctcatcagccagtgtggaaaaccagtggtaaatgtcagctacaggccagcttatgaccagggtggcccagttgaaatccttcccttcctctaccttggaagtgcctaccatgcatccaagtgcgagttcctcgccaacttgcacatcacagccctgctgaatgtctcccgacggacctccgaggcctgcatgacccacctacactacaaatggatccctgtggaagacagccacacggctgacattagctcccactttcaagaagcaatagacttcattgactgtgtcagggaaaagggaggcaaggtcctggtccactgtgaggctgggatctcccgttcacccaccatctgcatggcttaccttatgaagaccaagcagttccgcctgaaggaggccttcgattacatcaagcagaggaggagcatggtctcgcccaactttggcttcatgggccagctcctgcagtacgaatctgagatcctgccctccacgcccaacccccagcctccctcctgccaaggggaggcagcaggctcttcactgataggccatttgcagacactgagccctgacatgcagggtgcctactgcacattccctgcctcggtgctggcaccggtgcctacccactcaacagtctcagagctcagcagaagccctgtggcaacggccacatcctgctaaaactgggatggaggaatcggcccagccccaagagcaactgtgatttttgtttttaagactcatggacatttcatacctgtgcaatactgaagacctcattctgtcatgctgccccagtgagatagtgagtggtcaccaggcttgcaaatgaacttcagacggacctcagggtaggttctcgggactgaaggaaggccaagccattacgggagcacagcatgtgctgactactgtacttccagacccctgccctcttgggactgcccagtccttgcacctcagagttcgccttttcatttcaagcataagccaataaatacctgcagcaacgtgggagaaagaagttgctggaccaggagaaaaggcagttatgaagccaattcattttgaaggaagcacaatttccaccttattttttgaactttggcagtttcaatgtctgtctctgttgcttcggggcataagctgatcaccgtctagttgggaaagtcaccctacagggtttgtagggacatgatcagcatcctgatttgaaccctgaaatgttgtgtagacaccctcttgggtccaatgaggtagttggttgaagtagcaagatgttggcttttctggattttttttgccatgggttcttcactgaccttggactttggcatgattcttagtcatacttgaacttgtctcattccacctcttctcagagcaactcttcctttgggaaaagagttcttcagatcatagaccaaaaaagtcataccttcgaggtggtagcagtagattccaggaggagaagggtacttgctaggtatcctgggtcagtggcggtgcaaactggtttcctcagctgcctgtccttctgtgtgcttatgtctcttgtgacaattgttttcctccctgcccctggaggttgtcttcaactgtggacttctgggatttgcagattttgcaacgtggtactacttttttttctttttgtctgttagttatttctccaggggaaaaggcaataattttctaagacccgtgtgaatgtgaagaaaagcagtatgttactggttgttgttgttgttcttgttttttatatgtaaaataaaaatagtgaaaggag

SEQ ID NO: 14 - DNA, 976 bp, Homo sapiens
cccggaacctggcgcaactcctagagcggtccttggggagacgcgggtcccagtcctgcggctcctactggggagtgcgctggtcggaagattgctggactcgctgaagagagactacgcaggaaagccccagccacccatcaaatcagagagaaggaatccaccttcttacgctatggcaggtaagaaagtactcattgtctatgcacaccaggaacccaagtctttcaacggatccttgaagaatgtggctgtagatgaactgagcaggcagggctgcaccgtcacagtgtctgatttgtatgccatgaactttgagccgagggccacagacaaagatatcactggtactctttctaatcctgaggttttcaattatggagtggaaacccacgaagcctacaagcaaaggtctctggctagcgacatcactgatgagcagaaaaaggttcgggaggctgacctagtgatatttcagttcccgctgtactggttcagcgtgccggccatcctgaagggctggatggatagggtgctgtgccagggctttgcctttgacatcccaggattctacgattccggtttgctccagggtaaactagcgctcctttccgtaaccacgggaggcacggccgagatgtacacgaagacaggagtcaatggagattctcgatacttcctgtggccactccagcatggcacattacacttctgtggatttaaagtccttgcccctcagatcagctttgctcctgaaattgcatccgaagaagaaagaaaggggatggtggctgcgtggtcccagaggctgcagaccatctggaaggaagagcccatcccctgcacagcccactggcacttcgggcaataactctgtggcacgtgggcatcacgtaagcagcacactaggaggcccaggcgcaggcaaagagaagatggtgctgtcatgaaataaaattacaacatagctacctgg

SEQ ID NO: 15 - DNA, 7560 bp, Homo sapiens
accggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgaatccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtagatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtgaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtttggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcggctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacttaacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacggcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctcaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattggagggaccactgggtgagagaggaataaggcggcccagagcgaggaaaggattttaccaaagcatcaatacaaccagcccaaccatcggtccacacctgggcatttggtgagaatcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgaaaaaaaaccggaattc

SEQ ID NO: 16 - DNA, 2232 bp, Homo sapiens
cttccccttctctgccctgctccaggcaccaggctctttccccttcagtgtctcagaggaggggacggcagcaccatggacccccgcttgtccactgtccgccagacctgctgctgcttcaatgtccgcatcgcaaccaccgccctggccatctaccatgtgatcatgagcgtcttgttgttcatcgagcactcagtagaggtggcccatggcaaggcgtcctgcaagctctcccagatgggctacctcaggatcgctgacctgatctccagcttcctgctcatcaccatgctcttcatcatcagcctgagcctactgatcggcgtagtcaagaaccgggagaagtacctgctgcccttcctgtccctgcaaatcatggactatctcctgtgcctgctcaccctgctgggctcctacattgagctgcccgcctacctcaagttggcctcccggagccgtgctagctcctccaagttccccctgatgacgctgcagctgctggacttctgcctgagcatcctgaccctctgcagctcctacatggaagtgcccacctatctcaacttcaagtccatgaaccacatgaattacctccccagccaggaggatatgcctcataaccagttcatcaagatgatgatcatcttttccatcgccttcatcactgtccttatcttcaaggtctacatgttcaagtgcgtgtggcggtgctacagattgatcaagtgcatgaactcggtggaggagaagagaaactccaagatgctccagaaggtggtcctgccgtcctacgaggaagccctgtctttgccatcgaagaccccagaggggggcccagcaccacccccatactcagaggtgtgaccctcgccaggccccagccccagtgctgggaggggtggagctgcctcataatctgcttttttgctttggtggcccctgtggcctgggtgggccctcccgcccctccctggcaggacaatctgcttgtgtctccctcgctggcctgctcctcctgcagggcctgtgagctgctcacaactgggtcaacgctttaggctgagtcactcctcgggtctctccataattcagcccaacaatgcttggtttatttcaatcagctctgacacttgtttagacgattggccattctaaagttggtgagtttgtcaagcaactatcgacttgatcagttcagccaagcaactgacaaatcaaaaacccacttgtcagttcagtaaaataatttggtcaaacaacagtctattgcattgatttataaatagttgtcagttcacatagcaatttaatcaagtaatcattaattagttaccccctatatataaatatatgtaatcaatttcttcaaatagcttgcttacatgataatcaattagccaaccatgagtcatttagaatagtgataaatagaatacacagaatagtgatgaaattcaatttaaaaaatcacgttagcctccaaaccatttaattcaaatgaacccatcaactggatgccaactctggcgaatgtaggacctctgagtggctgtataattgttaattcaaatgaaattcatttaaacagttgacaaactgtcattcaacaattagctccaggaaataacagttatttcatcataaaacagtcccttcaaacacacaattgttctgctgaagagttgtcatcaacaatccaatgctcacctattcagttgctctgtggtcagtgtggctgcatagcagtggattccatgaaaggagtcattttagtgatgagctgccagtccattcccaggccaggctgtcgctggccatccattcagtcgattcagtcataggcgaatctgttctgcccgaggcttgtggtcaagcaaaaattcagccctgaaatcaggcacatctgttcgttggactaaacccacaggttagttcagtcaaagcaggcaacccccttgtgggcactgaccctgccactggggtcatggcggttgtggcagctggggaggtttggccccaacagccctcctgtgcctgcttccctgtgtgtcggggtcctccagggagctgacccagaggtggaggccacggaggcagggtctctggggactgtcggggggtacagagggagaaggctctgcaagagctccctggcaatacccccttgtgtaattgctttgtgtgcgacagggaggaagtttcaataaagcaacaacaagcttcaaggaattc

SEQ ID NO: 17 - DNA, 2709 bp, Homo sapiens
gggaatagcagaataggagcaagccagcactagtcagctaactaagtgactcaaccaaggccttttttccttgttatctttgcagatacttcattttcttagcgtttctggagattacaacatcctgcggttccgtttctgggaactttactgatttatctcccccctcacacaaataagcattgattcctgcatttctgaagatctcaagatctggactactgttgaaaaaatttccagtgaggctcacttatgtctgtaaagatgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtgcatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacagttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccagcttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaacacaatcaactgtgaggaaggagataaactgaaactcaccagctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaaaatctggatgtcattgacgataatgtttatggagataaggtctaagtccctaaaaaaatgtacatatacctggttgaaatacaacactatacatacacaccaccatatatactagctgttaatcctatggaatgggggtattgggagtgcttttttaatttttcatagtttttttttaataaaatggcatattttgcatctacaacttctataataagaaaaaataaataaacattatcttttttgtgaaaaaaa

SEQ ID NO: 18 - DNA, 1722 bp, Homo sapiens
gcgggcggttcagccatgaggctggctgtgcttttctcgggggccctgctggggctactggcagcccaggggacagggaatgactgtcctcacaaaaaatcagctactttgctgccatccttcacggtgacacccacggttacagagagcactggaacaaccagccacaggactaccaagagccacaaaaccaccactcacaggacaaccaccacaggcaccaccagccacggacccacgactgccactcacaaccccaccaccaccagccatggaaacgtcacagttcatccaacaagcaatagcactgccaccagccagggaccctcaactgccactcacagtcctgccaccactagtcatggaaatgccacggttcatccaacaagcaacagcactgccaccagcccaggattcaccagttctgcccacccagaaccacctccaccctctccgagtcctagcccaacctccaaggagaccattggagactacacgtggaccaatggttcccagccctgtgtccacctccaagcccagattcagattcgagtcatgtacacaacccagggtggaggagaggcctggggcatctctgtactgaaccccaacaaaaccaaggtccagggaagctgtgagggtgcccatccccacctgcttctctcattcccctatggacacctcagctttggattcatgcaggacctccagcagaaggttgtctacctgagctacatggcggtggagtacaatgtgtccttcccccacgcagcaaagtggacattctcggctcagaatgcatcccttcgagatctccaagcacccctggggcagagcttcagttgcagcaactcgagcatcattctttcaccagctgtccacctcgacctgctctccctgaggctccaggctgctcagctgccccacacaggggtctttgggcaaagtttctcctgccccagtgaccggtccatcttgctgcctctcatcatcggcctgatccttcttggcctcctcgccctggtgcttattgctttctgcatcatccggagacgcccatccgcctaccaggccctctgagcatttgcttcaaaccccagggcactgagggggtttggggtgtggtgggggggtacccttatttcctcgacacgccgctggctcaaagacaatgttattttccttccctttcttgaagaacaaaaagaaagccgggcatgacggctcatgcctgtaatcccagcactttgggaggctgaggcaggtggatcactggaggtcaggtctttgaggccagccctagccaacatggtgtaaacactgtctctactaaaaatacaattagccaggtgtggcggcgtaatcccatgctaacctgtaatcccagctacttgggaggctgaggcagagctgcttgaaccctggaagtggaggttgcagtgagcctgtcatcgctccactgagccaagatcgctcccactgcactccagcctgggcgacagagccagactgtctcaaataaataaatatgagataatgcagtcgggagaagggagggagagaattttattaaatgtgacgaactgcccccccccccccccccagcaggagagcagcaaaatttatgtaaatctttgacggggttttccttgctcctgccaggattaaaagtccatgagtttcttgctcaaaaaaaaaaaaaaaaaaaaaaaaaaaaaaaaa

SEQ ID NO: 19 - DNA, 1522 bp, Homo sapiens
caggtgtggattccgccggtgaaggctgaaggcagctaccttaaagatgccgggatccgcagcgaagggctcggagttgtcagagaggatcgagagcttcgtggagaccctgaagcggggtggtgggccgcgcagctccgaggaaatggctcgggagaccctagggttgctgcgccagatcatcacggaccaccgctggagcaacgcgggggagctgatggagctgatccgcagagagggcaggaggatgacggccgctcagccctccgagaccaccgtgggcaacatggtgcggagagtgctcaagattatccgggaggagtatggcagactccatggacgcagcgacgagagtgatcagcaggagtccctgcacaaactgttgacatccggaggcctaaacgaggatttcagcttccattatgcccaactccagtccaacatcattgaggcgattaatgagctgctagtggagctggaagggacaatggagaacattgcagcccaggctctggagcacattcactccaatgaggtgatcatgaccattggcttctcccgaacagtagaggccttcctcaaagaggctgcccgaaagaggaaattccatgtcattgtagcagagtgtgctcctttctgccagggtcatgaaatggctgtgaatttgtccaaagcaggtattgagacaactgtcatgactgatgctgccatttttgccgttatgtcaagagtcaacaaggtgatcattggcacgaagaccatcctggccaatggggccctgagagctgtgacaggaactcacactctggcactggcagcaaaacaccattccaccccactcatcgtctgtgcacctatgttcaaactttctccacagttccccaatgaagaagactcatttcataagtttgtggctcctgaagaagtcctgccattcacagaaggggacattctggagaaggtcagcgtgcattgccctgtgtttgactacgttcccccagagctcattaccctctttatctccaacattggtgggaatgcaccttcctacatctaccgcctgatgagtgaactctaccatcctgatgatcatgttttatgaccgaccacacgtgtcctaagcagattgcttaggcagatacagaatgaagaggagacttgagtgttgctgctgaagcacatccttgcaatgtgggagtgcacaggagtccacctaaaaaaaaaatccttgatactgttgcctgcctttttagtcaccccgtaacaagggcacacatccagcactgtgtcttgcctttcagatcttaacagagcagcagggcttaacttgttgattttggagcctcttagtgacctggttgcgtctgtgtcaggaacttaaactttctggttcagtagtgtgttaaacataacactgaataccttactgggatacagatttttgctcagaaatggctatgacactttttctaggctctaccaataaaagccacttgaaggttcaaaaaaaaaaaaaaaaaaa

SEQ ID NO: 20 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKINAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 21 - protein, 110 aa, Homo sapiens
MSMTDLLNAEDIKKAVGAFSATDSFDHKKFFQMVGLKKKSADDVKKVFHMLDKDKSGFIEEDELGFILKGFSPDARDLSAKETKMLMAAGDKDGDGKIGVDEFSTLVAES

SEQ ID NO: 22 - protein, 1722 aa, Homo sapiens
MAGVGPGGYAAEFVPPPECPVFEPSWEEFTDPLSFIGRIRPLAEKTGICKIRPPKDWQPPFACEVKSFRFTPRVQRLNELEAMTRVRLDFLDQLAKFWELQGSTLKIPVVERKILDLYALSKIVASKGGFEMVTKEKKWSKVGSRLGYLPGKGTGSLLKSHYERILYPYELFQSGVSLMGVQMPNLDLKEKVEPEVLSTDTQTSPEPGTRMNILPKRTRRVKTQSESGDVSRNTELKKLQIFGAGPKVVGLAMGTKDKEDEVTRRRKVTNRSDAFNMQMRQRKGTLSVNFVDLYVCMFCGRGNNEDKLLLCDGCDDSYHTFCLIPPLPDVPKGDWRCPKCVAEECSKPREAFGFEQAVREYTLQSFGEMADNFKSDYFNMPVHMVPTELVEKEFWRLVSSIEEDVIVEYGADISSKDFGSGFPVKDGRRKILPEEEEYALSGWNLNNMPVLEQSVLAHINVDISGMKVPWLYVGMCFSSFCWHIEDHWSYSINYLHWGEPKTWYGVPSHAAEQLEEVMRELAPELFESQPDLLHQLVTIMNPNVLMEHGVPVYRTNQCAGEFVVTFPRAYHSGFNQGYNFAEAVNFCTADWLPIGRQCVNHYRRLRRHCVFSHEELIFKMAADPECLDVGLAAMVCKELTLMTEEETRLRESVVQMGVLMSEEEVFELVPDDERQCSACRTTCFLSALTCSCNPERLVCLYHPTDLCPCPMQKKCLRYRYPLEDLPSLLYGVKVRAQSYDTWVSRVTEALSANFNHKKDLIELRVMLEDAEDRKYPENDLFRKLRDAVKEAETCASVAQLLLSKKQKHRQSPDSGRTRTKLTVEELKAFVQQLFSLPCVISQARQVKNLLDDVEEFHERAQEAMMDETPDSSKLQMLIDMGSSLYVELPELPRLKQELQQARWLDEVRLTLSDPQQVTLDVMKKLIDSGVGLAPHHAVEKAMAELQELLTVSERWEEKAKVCLQARPRHSVASLESIVNEAKNIPAFLPNVLSLKEALQKAREWTAKVEAIQSGSNYAYLEQLESLSAKGRPIPVRLEALPQVESQVAAARAWRERTGRTFLKKNSSHTLLQVLSPRTDIGVYGSGKNRRKKVKELIEKEKEKDLDLEPLSDLEEGLEETRDTAMVVAVFKEREQKEIEAMHSLRAANLAKMTMVDRIEEVKFCICRKTASGFMLQCELCKDWFHNSCVPLPKSSSQKKGSSWQAKEVKFLCPLCMRSRRPRLETILSLLVSLQKLPVRLPEGEALQCLTERAMSWQDRARQALATDELSSALAKLSVLSQRMVEQAAREKTEKIISAELQKAAANPDLQGHLPSFQQSAFNRVVSSVSSSPRQTMDYDDEETDSDEDIRETYGYDMKDTASVKSSSSLEPNLFCDEEIPIKSEEVVTHMWTAPSFCAEHAYSSASKSCSQVFFGKGSSTPRKQPRKSPLVPRSLEPPVLELSPGAKAQLEELMMVGDLLEVSLDETQHIWRILQATHPPSEDRFLHIMEDDSMEEKPLKVKGKDSSEKKRKRKLEKVEQLFGEGKQKSKELKKMDKPRKKKLKLGADKSKKLNKLAKKLAKEEERKKKKEKAAAAKVELVKESTEKKREKKVLDIPSKYDWSGAEESDDENAVCAEPDCQRPCKDKGVVFVTKKREIKNISFKSVLCDCFSKKVDWVQCDGGCDEWFHRVCVGVSPEMAENEDYICINCAKKQGPVSPGPAPPPSFIMSYKLPMEDLKETS

SEQ ID NO: 23 - protein, 373 aa, Homo sapiens
MGSQVSVESGALHVVIVGGGFGGIAAASQLQALNVPFMLVDMKDSFHHNVAALRASVETGFAKKTFISYSVTFKDNFRQGLVVGIDLKNQMVLLQGGEALPFSHLILATGSTGPFPGKFNEVSSQQAAIQAYEDMVRQVQRSRFIVVVGGGSAGVEMAAEIKTEYPEKEVTLIHSQVALADKELLPSVRQEVKEILLRKGVQLLLSERVSNLEELPLNEYREYIKVQTDKGTEVATNLVILCTGIKINSSAYRKAFESRLASSGALRVNEHLQVEGHSNVYAIGDCADVRTPKMAYLAGLHANIAVANIVNSVKQRPLQAYKPGALTFLLSMGRNDGVGQISGFYVGRLMVRLTKSRDLFVSTSWKTMRQSPP

SEQ ID NO: 24 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 25 - protein, 422 aa, Homo sapiens
MNSGHSFSQTPSASFHGAGGGWGRPRSFPRAPTVHGGAGGARISLSFTTRSCPPPGGSWGSGRSSPLLGGNGKATMQNLNDRLASYLEKVRALEEANMKLESRILKWHQQRDPGSKKDYSQYEENITHLQEQIVDGKMTNAQIILLIDNARMAVDDFNLKYENEHSFKKDLEIEVEGLRRTLDNLTIVTTDLEQEVEGMRKELILMKKHHEQEMEKHHVPSDFNVNVKVDTGPREDLIKVLEDMRQEYELIIKKKHRDLDTWYKEQSAAMSQEAASPATVQSRQGDIHELKRTFQALEIDLQTQYSTKSALENMLSETQSRYSCKLQDMQEIISHYEEELTQLRHELERQNNEYQVLLGIKTHLEKEITTYRRLLEGESEGTREESKSSMKVSATPKIKAITQETINGRLVLCQVNEIQKHA

SEQ ID NO: 26 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 27 - protein, 472 aa, Homo sapiens
MAGGEAGVTLGQPHLSRQDLTTLDVTKLTPLSHEVISRQATINIGTIGHVAHGKSTVVKAISGVHTVRFKNELERNITIKLGYANAKIYKLDDPSCPRPECYRSCGSSTPDEFPTDIPGTKGNFKLVRHVSFVDCPGHDILMATMLNGAAVMDAALLLIAGNESCPQPQTSEHLAAIEIMKLKHILILQNKIDLVKESQAKEQYEQILAFVQGTVAEGAPIIPISAQLKYNIEVVCEYIVKKIPVPPRDFTSEPRLIVIRSFDVNKPGCEVDDLKGGVAGGSILKGVLKVGQEIEVRPGIVSKDSEGKLMCKPIFSKIVSLFAEHNDLQYAAPGGLIGVGTKIDPTLCRADRMVGQVLGAVGALPEIFTELEISYFLLRRLLGVRTEGDKKAAKVQKLSKNEVLMVNIGSLSTGGRVSAVKADLGKIVLTNPVCTEVGEKIALSRRVEKHWRLIGWGQIRRGVTIKPTVDDD

SEQ ID NO: 28 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 29 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 30 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 31 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 32 - protein, 384 aa, Homo sapiens
MKVTSLDGRQLRKMLRKEAAARCVVLDCRPYLAFAASNVRGSLNVNLNSVVLRRARGGAVSARYVLPDEAARARLLQEGGGGVAAVVVLDQGSRHWQKLREESAARVVLTSLLACLPAGPRVYFLKGGYETFYSEYPECCVDVKPISQEKIESERALISQCGKPVVNVSYRPAYDQGGPVEILPFLYLGSAYHASKCEFLANLHITALLNVSRRTSEACMTHLHYKWIPVEDSHTADISSHFQEAIDFIDCVREKGGKVLVHCEAGISRSPTICMAYLMKTKQFRLKEAFDYIKQRRSMVSPNFGFMGQLLQYESEILPSTPNPQPPSCQGEAAGSSLIGHLQTLSPDMQGAYCTFPASVLAPVPTHSTVSELSRSPVATATSC

SEQ ID NO: 33 - protein, 231 aa, Homo sapiens
MAGKKVLIVYAHQEPKSFNGSLKNVAVDELSRQGCTVTVSDLYAMNFEPRATDKDITGTLSNPEVFNYGVETHEAYKQRSLASDITDEQKKVREADLVIFQFPLYWFSVPAILKGWMDRVLCQGFAFDIPGFYDSGLLQGKLALLSVTTGGTAEMYTKTGVNGDSRYFLWPLQHGTLHFCGFKVLAPQISFAPEIASEEERKGMVAAWSQRLQTIWKEEPIPCTAHWHFGQ

SEQ ID NO: 34 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGLDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAQALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 35 - protein, 262 aa, Homo sapiens
MDPRLSTVRQTCCCFNVRIATTALAIYHVIMSVLLFIEHSVEVAHGKASCKLSQMGYLRIADLISSFLLITMLFIISLSLLIGVVKNREKYLLPFLSLQIMDYLLCLLTLLGSYIELPAYLKLASRSRASSSKFPLMTLQLLDFCLSILTLCSSYMEVPTYLNFKSMNHMNYLPSQEDMPHNQFIKMMIIFSIAFITVLIFKVYMFKCVWRCYRLIKCMNSVEEKRNSKMLQKVVLPSYEEALSLPSKTPEGGPAPPPYSEV

SEQ ID NO: 36 - protein, 729 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVHATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLNTINCEEGDKLKLTSFELAPKSGNTGELRSVIHSHIKVIKTRKNKKDILNPDSSMETSPDFFF

SEQ ID NO: 37 - protein, 354 aa, Homo sapiens
MRLAVLFSGALLGLLAAQGTGNDCPHKKSATLLPSFTVTPTVTESTGTTSHRTTKSHKTTTHRTTTTGTTSHGPTTATHNPTTTSHGNVTVHPTSNSTATSQGPSTATHSPATTSHGNATVHPTSNSTATSPGFTSSAHPEPPPPSPSPSPTSKETIGDYTWTNGSQPCVHLQAQIQIRVMYTTQGGGEAWGISVLNPNKTKVQGSCEGAHPHLLLSFPYGHLSFGFMQDLQQKVVYLSYMAVEYNVSFPHAAKWTFSAQNASLRDLQAPLGQSFSCSNSSIILSPAVHLDLLSLRLQAAQLPHTGVFGQSFSCPSDRSILLPLIIGLILLGLLALVLIAFCIIRRRPSAYQAL

SEQ ID NO: 38 - protein, 351 aa, Homo sapiens
MPGSAAKGSELSERIESFVETLKRGGGPRSSEEMARETLGLLRQIITDHRWSNAGELMELIRREGRRMTAAQPSETTVGNMVRRVLKIIREEYGRLHGRSDESDQQESLHKLLTSGGLNEDFSFHYAQLQSNIIEAINELLVELEGTMENIAAQALEHIHSNEVIMTIGFSRTVEAFLKEAARKRKFHVIVAECAPFCQGHEMAVNLSKAGIETTVMTDAAIFAVMSRVNKVIIGTKTILANGALRAVTGTHTLALAAKHHSTPLIVCAPMFKLSPQFPNEEDSFHKFVAPEEVLPFTEGDILEKVSVHCPVFDYVPPELITLFISNIGGNAPSYIYRLMSELYHPDDHVL